SEQUENCE LISTING (12 sequences)

SEQ ID NO: 1 - protein, 777 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 2 - DNA, 2334 bp, Synthetic construct
  source 1..2334: mol_type = other DNA
atggacagcaaggaatccctaactccggggagagaggagaacccatcctccgtgcttgcgcaggagagaggagatgttatggacttctacaaaaccctaagagggggtgccacagtaaaagtcagtgcaagttcaccatccttagcggtagcttcccaatcagatagtaagcaaaggcgtctgctagtcgatttccctaagggaagtgttagcaatgcacaacaaccggatttatctaaagccgtctctttatccatgggcttgtacatgggtgaaacggagacaaaggtcatgggtaacgacttaggcttcccccagcaaggccagataagtctatcctcaggagagaccgaccttaagcttttagaagaaagcatagcaaacctgaacaggagtacttctgtacctgagaatcccaagagtagcgcatcaaccgccgtgtccgctgcgccaacagagaaagaatttccgaaaacgcatagtgatgtgtcttctgagcaacaacacttgaaaggacaaactggtacgaatggtgggaatgtgaagctttacacaactgatcagtctacatttgatatactacaggacttggaatttagttcaggcagtccagggaaggaaaccaatgaatctccgtggcgttcagacttactgatagacgagaattgtttacttagtccgcttgctggagaggacgactctttcttactggagggtaacagcaatgaggactgtaagccccttattctgcctgataccaaaccgaagattaaagataatggtgatctagtgctatcctcacccagcaatgtgaccctgccacaagtcaagaccgagaaagaagatttcattgaactttgtactccaggtgtgataaaacaggaaaaactgggcactgtgtattgtcaggctagcttcccaggcgctaacatcatcggaaataaaatgagcgcaatcagcgtccacggcgtgtctacatctggtggccagatgtatcattatgacatgaataccgcttccctttcacagcaacaagatcaaaaaccgatattcaacgtgataccccctataccagtgggctccgaaaactggaatcgttgtcaaggttctggagatgataatttgactagcttggggactttaaactttccaggtcgtacagttttctccaatggatacagtagcccctcaatgagaccagacgtatcaagtccaccatcctcctcatccacagccactacgggtccgccgccgaaactatgtttagtgtgcagcgatgaggcatcaggatgccattatggcgtccttacatgcgggagctgtaaggtgttttttaagagagcggtcgaagggcaacataactacttatgcgcgggcagaaacgattgtatcatcgacaagataaggaggaaaaattgtcctgcatgcaggtacaggaaatgccttcaggctggcatgaatctagaggctcgtaagacgaagaaaaaaataaaaggtattcaacaggcaactacgggcgtcagccaagaaacgtctgaaaatccagggaataaaactatcgtaccggcgacactaccgcaattgacgcctactctagtatctttgttagaagtgatagaacccgaagtcctgtatgcgggttatgactccagcgtaccagatagtacttggcgtataatgactacactgaacatgttaggcggaaggcaggtcattgccgcggttaaatgggcgaaagcgatcccaggcttcaggaacctacatctagacgatcaaatgacattgttacaatacagctggatgttcctgatggcgttcgcgttgggatggaggagctaccgtcaaagcagtgcaaatttactgtgttttgcccccgatctgattatcaacgaacagcgtatgaccttgccatgcatgtacgaccaatgcaaacatatgctttacgtctcaagtgagttacacagacttcaggtctcttacgaggagtatctttgtatgaagacattgttgcttctgagctccgtgcccaaggacggtttgaaatctcaggagttgtttgacgaaattcgtatgacgtacataaaagagctggggaaagccatagtcaagagagagggcaacagtagccagaactggcaaagattctaccaacttaccaagctgcttgattcaatgcacgaagtcgtcgaaaacctgcttaactattgcttccagacgtttctagacaaaaccatgtccatcgagttccccgagatgttggcagagatcattacaaatcagatccccaaatacagcaatggtaacataaagaagctactgtttcatcagaaataa

SEQ ID NO: 3 - protein, 487 aa, Homo sapiens
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIVHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILHIRS

SEQ ID NO: 4 - DNA, 1464 bp, Synthetic construct
  source 1..1464: mol_type = other DNA
atgtccttgccaaactcttcttgtttgttggaagataagatgtgcgaaggtaacaagactactatggcttctccacaattgatgccattggttgttgttttgtctaccatttgcttggttaccgttggtttgaatttgttggtcttgtacgctgtgagatccgaaagaaaattgcatactgttggcaacctgtacatcgtttctttgtctgttgctgatttgatcgttggtgctgttgttatgccaatgaacatcttgtacctgttgatgtctaaatggtctttgggtagaccattgtgtttgttctggttgtctatggattacgttgcttctaccgcttctatcttctccgttttcattttgtgcatcgacagatacaggtctgttcaacaaccattgagatacttgaagtacagaactaagactagagcttccgctactattttaggtgcttggtttttgtctttcttgtgggtcattccaatcttaggttggaaccattttatgcaacaaaccagcgttagacgtgaagataagtgcgaaactgatttctacgatgttacctggttcaaggttatgaccgccattattaacttctacttgccaaccttgttgatgttgtggttttacgctaaaatctacaaggccgttagacaacattgccaacatagagaattgatcaacagatccttgccatccttcagcgaaatcaagttaagaccagaaaatccaaagggtgatgctaagaaacctggtaaagaatctccatgggaagtcttgaaaagaaagccaaaagatgctggtggtggttctgttttgaaatctccatctcaaaccccaaaagagatgaagtctccagttgttttctcacaagaagatgatagagaagtcgacaagttgtactgtttcccattggatatcgttcatatgcaagctgctgctgaaggttcttctagagattatgttgccgttaacagatcccacggtcaattgaaaactgatgaacaaggtttgaatacccatggtgcctctgaaatttctgaagatcaaatgttgggtgactcccagtctttttctagaactgattctgataccactactgaaactgctccaggtaaaggtaaattgagatctggttctaataccggcttggactacattaagtttacttggaagagactgaggtcccactctagacaatatgtttctggtttacacatgaacagggaaagaaaagctgctaagcaattgggtttcattatggctgcctttattttgtgctggatcccttacttcattttcttcatggttatcgctttctgcaagaactgttgtaacgaacacttgcacatgttcactatttggttgggttacatcaactccactttgaacccattgatctacccattgtgcaacgagaacttcaaaaagaccttcaagagaatcttgcacatcagatcctaa

SEQ ID NO: 5 - DNA, 109 bp, Synthetic construct
  source 1..109: mol_type = other DNA
aaagtcagaacagcatgttctgatcaaatctagaagatccaaagtcagaacagcatgttctgatcaaactcgagcagatccgccaggcgtgtatatatagcgtggatgg

SEQ ID NO: 6 - DNA, 202 bp, Saccharomyces cerevisiae
  source 1..202: mol_type = genomic DNA
ccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacgcaaacacaaatacacacactaa

SEQ ID NO: 7 - DNA, 1416 bp, Synthetic construct
  source 1..1416: mol_type = other DNA
atggggtgtacagtgagtacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatatgggcagatgccatacaatcaatgaaaattttgattattcaggccagaaaactaggtattcaacttgactgtgatgatccgatcaacaataaagatttgtttgcatgcaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgctgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgttttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcaccgatctaatcatccagcaaaaccttaaagaatataatctggtg

SEQ ID NO: 8 - protein, 472 aa, Synthetic construct
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKEYNLV

SEQ ID NO: 9 - DNA, 1419 bp, Saccharomyces cerevisiae
  source 1..1419: mol_type = genomic DNA
atggggtgtacagtgagtacgcaaacaataggagacgaaagtgatccttttctacagaacaaaagagccaatgatgtcatcgagcaatcgttgcagctggagaaacaacgtgacaagaatgaaataaaactgttactattaggtgccggtgagtcaggtaaatcaacggttttaaaacaattaaaattattacatcaaggcggtttctcccatcaagaaaggttacagtatgctcaagtgatatgggcagatgccatacaatcaatgaaaattttgattattcaggccagaaaactaggtattcaacttgactgtgatgatccgatcaacaataaagatttgtttgcatgcaagagaatactgctaaaggctaaagctttagattatatcaacgccagtgttgccggtggttctgattttctaaatgattatgtactgaagtactcagaaaggtatgaaactaggaggcgtgttcagagtaccggacgagcaaaagctgctttcgatgaagacggaaatatttctaatgtcaaaagtgacactgacagagatgctgaaacggtgacgcaaaatgaggatgctgatagaaacaacagtagtagaattaacctacaggatatttgcaaggacttgaaccaagaaggcgatgaccagatgtttgttagaaaaacatcaagggaaattcaaggacaaaatagacgaaatcttattcacgaagacattgctaaggcaataaagcaactttggaataacgacaaaggtataaagcagtgttttgcacgttctaatgagtttcaattggagggctcagctgcatactactttgataacattgagaaatttgctagtccgaattatgtctgtacggatgaagacattttgaagggccgtataaagactacaggcattacagaaaccgaatttaacatcggctcgtccaaattcaaggttctcgacgctggtgggcagcgttctgaacgtaagaagtggattcattgtttcgaaggaattacagcagttttatttgttttagcaatgagtgaatacgaccagatgttgtttgaggatgaaagagtgaacagaatgcatgaatcaataatgctatttgacacgttattgaactctaagtggttcaaagatacaccgtttattttgtttttaaataaaattgatttgttcgaggaaaaggtaaaaagcatgcccataagaaagtactttcctgattaccagggacgtgtcggcgatgcagaagcgggtctaaaatattttgagaagatatttttgagcttgaataagacaaacaaaccaatctacgtgaaacgaacctgcgctaccgatacccaaactatgaagttcgtattgagtgcagtcaccgatctaatcatccagcaaaaccttaaaaaaattggtattatatga

SEQ ID NO: 10 - protein, 472 aa, Saccharomyces cerevisiae
MGCTVSTQTIGDESDPFLQNKRANDVIEQSLQLEKQRDKNEIKLLLLGAGESGKSTVLKQLKLLHQGGFSHQERLQYAQVIWADAIQSMKILIIQARKLGIQLDCDDPINNKDLFACKRILLKAKALDYINASVAGGSDFLNDYVLKYSERYETRRRVQSTGRAKAAFDEDGNISNVKSDTDRDAETVTQNEDADRNNSSRINLQDICKDLNQEGDDQMFVRKTSREIQGQNRRNLIHEDIAKAIKQLWNNDKGIKQCFARSNEFQLEGSAAYYFDNIEKFASPNYVCTDEDILKGRIKTTGITETEFNIGSSKFKVLDAGGQRSERKKWIHCFEGITAVLFVLAMSEYDQMLFEDERVNRMHESIMLFDTLLNSKWFKDTPFILFLNKIDLFEEKVKSMPIRKYFPDYQGRVGDAEAGLKYFEKIFLSLNKTNKPIYVKRTCATDTQTMKFVLSAVTDLIIQQNLKKIGII

SEQ ID NO: 11 - DNA, 311 bp, Synthetic construct
  source 1..311: mol_type = other DNA
aaagtcagaacagcatgttctgatcaaatctagaagatccaaagtcagaacagcatgttctgatcaaactcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacgcaaacacaaatacacacactaa

SEQ ID NO: 12 - DNA, 645 bp, Synthetic construct
  source 1..645: mol_type = other DNA
tcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacaccgaaggtgcctgggtgaacaggtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcga

(19) United States
(12) Patent Application Publication
(10) Pub. No.: US 2026/0317692 A1
(43) Pub. Date: Oct. 15, 2026

(54) ENGINEERED YEAST STRAINS FOR BIOSENSING AND TAILORED BIOLOGICAL RESPONSE

(71) Applicant: Verb Biotics, Boston, MA (US)

(72) Inventors: Todd Beckman, Boston, MA (US); Camille Delebecque, Portsmouth, NH (US); Noah Zimmerman, Chapel Hill, NC (US)

(73) Assignee: Verb Biotics, Boston, MA (US)

(21) Appl. No.: 19/448,798

(22) Filed: Jan. 14, 2026

Related U.S. Application Data
(60) Provisional application No. 63/745,159, filed on Jan. 14, 2025.

(51) Int. Cl. C12N 1/16; A61K 31/7088; A61K 36/064; A61K 38/17; A61K 49/00; C12R 1/865

(52) U.S. Cl. CPC C12N 1/16; A61K 31/7088; A61K 36/064; A61K 38/1796; A61K 49/00; A61K 2123/00; C12R 2001/865

(57) ABSTRACT

Provided herein are biosensing compositions and associated methods of making and using biosensing compositions to bind, detect, and/or reduce the levels of a target molecule in or on the tissue of a subject. Also disclosed are methods to reduce a sign or symptom of a disease and/or disorder, and methods to maintain a healthy biological function within a tissue or on a tissue of a subject using the biosensing compositions. Also provided are yeast cells engineered to bind and/or interact with and detect a target molecule in or on the tissue of a subject. Following interaction with the target molecule, the engineered yeast can produce and secrete a response factor in or on a tissue, reducing the levels, activity and/or effects of a target molecule present within or on the tissue at or above a specific threshold concentration. Probiotics and pharmaceutical compositions comprising biosensing compositions are also provided.

### CROSS-REFERENCE TO RELATED APPLICATIONS

This application claims priority to U.S. Provisional Application No. 63/745,159, filed Jan. 14, 2025, the contents of which is incorporated by reference herein in its entirety.

### STATEMENT REGARDING THE SEQUENCE LISTING

The instant application contains an electronic Sequence Listing which has been submitted in xml (ST.26) format via USPTO Patent Center, herein incorporated by reference in its entirety. Said xml copy named “V118077 1130WO.xml” is 11,334 bytes in size, and was created on Jan. 13, 2026.

### FIELD OF THE INVENTION

The present invention relates to the field of molecular biology, microbiology, and biosensing. In particular, the present invention relates to using live microorganisms to detect, bind, and/or reduce levels and/or activity of a target molecule in or on a subject.

### BACKGROUND OF THE INVENTION

The present disclosure relates to compositions and methods for using an engineered yeast cell to detect target molecules in or on a tissue of a subject in vivo and subsequently release a response factor that modulates said target molecule, directly or indirectly. For example, in some instances, an engineered yeast cell can detect a target molecule on the skin or within the gastrointestinal tract of a subject. In some embodiments, the amount or the activity of the target molecule can be reduced. The response factor may act directly and/or indirectly on the target molecule. The target molecule can be a biomarker of a disease in said subject. In some instances, elevated levels of the target molecule are associated with a sign and/or symptom of a disease.

Provided herein are compositions and methods for improved monitoring, detection, and modulation of health and disease biomarkers in vivo. In particular, the compositions and methods are particularly effective for use on the skin or in the gastrointestinal tract of a subject.

### SUMMARY OF THE INVENTION

Biosensing compositions for detecting a target molecule in a subject, said compositions comprising a plurality of engineered yeast cells comprising a. a molecule responsive element comprising binding domain that interacts with said target molecule, or a polynucleotide comprising a nucleotide sequence encoding said molecule responsive element; and b. a nucleic acid molecule comprising a polynucleotide comprising a nucleotide sequence encoding a response factor, whereby said interaction of said target molecule with said binding domain results in production and/or secretion of said response factor in said engineered yeast cells.

In some embodiments of the biosensing composition, said molecule responsive element comprises a polypeptide.

In some embodiments of the biosensing composition, polypeptide comprises an amino acid sequence comprising said binding domain.

In some embodiments of the biosensing composition, said molecule responsive element comprises a nucleic acid molecule.

In some embodiments of the biosensing composition, said polypeptide comprises a protein, such as a fusion protein, a transmembrane protein, a receptor, and/or a functional fragment or variant thereof.

In some embodiments of the biosensing composition, sFaid receptor comprises a human glucocorticosteroid (GC) receptor, or a functional fragment or variant thereof having at least 80%, at least 85%, at least 90%, at least 95%, or at least 99% sequence identity to a human glucocorticosteroid (GC) receptor.

In some embodiments of the biosensing composition, said human glucocorticosteroid (GC) receptor comprises an amino acid sequence set forth as SEQ ID NO: 1, or a functional fragment or variant thereof having at least 80%, at least 85%, at least 90%, at least 95%, or at least 99% sequence identity to the amino acid sequence set forth as SEQ ID NO: 1; and/or said human glucocorticosteroid (GC) receptor is encoded by a nucleotide sequence set forth as SEQ ID NO: 2, or a variant thereof having at least 80%, at least 85%, at least 90%, at least 95%, or at least 99% sequence identity to the nucleotide sequence set forth as SEQ ID NO: 2.

In some embodiments of the biosensing composition, said receptor comprises a G-protein coupled receptor (GPCR) protein, or a functional fragment or variant thereof having at least 80%, at least 85%, at least 90%, at least 95%, or at least 99% sequence identity to a G-protein coupled receptor (GPCR) protein.

In some embodiments of the biosensing composition, said G-protein coupled receptor (GPCR) protein is a human G-protein coupled receptor (GPCR) protein.

In some embodiments of the biosensing composition, said G-protein coupled receptor (GPCR) protein, or functional fragment or variant thereof, comprises a histamine binding domain, such as a histamine binding ectodomain.

In some embodiments of the biosensing composition, said human G-protein coupled receptor (GPCR) protein comprises an amino acid sequence set forth as SEQ ID NO: 3, or a functional fragment or variant thereof having at least 80%, at least 85%, at least 90%, at least 95%, or at least 99% sequence identity to the amino acid sequence set forth as SEQ ID NO: 3; and/or said human G-protein coupled receptor (GPCR) protein is encoded by a nucleotide sequence set forth as SEQ ID NO: 4, or a variant thereof having at least 80%, at least 85%, at least 90%, at least 95%, or at least 99% sequence identity to the nucleotide sequence set forth as SEQ ID NO: 4.

In some embodiments of the biosensing composition, said yeast cells further comprise a polypeptide comprising an amino acid sequence encoding a modified yeast GPA1 protein, or a polynucleotide comprising a nucleotide sequence encoding a modified yeast GPA1 protein, wherein said polypeptide: a. comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10; b. comprises an amino acid sequence corresponding with residues 1-467 of SEQ ID NO: 10; c. comprises an amino acid sequence comprising one or more than one amino acid substitution compared to SEQ ID NO: 10, wherein said one or more than one amino acid substitution is selected from the following: K468E, I469Y, G470N, I471L, and I472V; d. comprises an amino acid sequence comprising the following amino acid substitutions: K468E, 1469Y, G470N, 1471L, and 1472V, compared to SEQ ID NO: 10; and/or e. comprises an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8; and/or said polynucleotide comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

In some embodiments of the biosensing composition, said polynucleotide comprising a nucleotide sequence encoding a response factor is operably fused to an activation domain.

In some embodiments of the biosensing composition, said activation domain comprises a promoter, such as a Ste-12-activated or a human glucocorticosteroid response element (HRE)-activated promoter.

In some embodiments of the biosensing composition, said response factor is produced upon interaction of STE12 or GC receptor to said promoter.

In some embodiments of the biosensing composition, said engineered yeast comprises mutation and/or deletion in at least one of the following genes: STE2, FAR1, SST2, and MTA-A.

In some embodiments of the biosensing composition, said nucleotide sequence encoding said molecule responsive element is codon-optimized for expression in a eukaryotic cell, such as a yeast cell.

In some embodiments of the biosensing composition, said polynucleotide comprising a nucleotide sequence encoding said molecule responsive element and said polynucleotide encoding said response factor are comprised within a single nucleic acid molecule.

In some embodiments of the biosensing composition, said nucleic acid molecule comprises a plasmid.

In some embodiments of the biosensing composition, said polynucleotide comprising a nucleotide sequence encoding said molecule responsive element and/or said polynucleotide encoding a response factor are integrated into the genome of said engineered yeast cells.

In some embodiments of the biosensing composition, said engineered yeast comprises Saccharomyces, such as Saccharomyces cerevisiae and/or Saccharomyces boulardii.

In some embodiments of the biosensing composition, said target molecule comprises a ligand, a protein, metabolite, a hormone, a nutrient, or small molecule, such as cortisol or histamine.

In some embodiments of the biosensing composition, said target molecule comprises cortisol or histamine.

In some embodiments of the biosensing composition, said engineered yeast cells produce an effective amount of said response factor in response to a threshold concentration of said target molecule.

In some embodiments of the biosensing composition, said target molecule is cortisol, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

In some embodiments of the biosensing composition, said target molecule is histamine, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

In some embodiments of the biosensing composition, said biosensing composition comprises a single species of microorganism.

In some embodiments of the biosensing composition, said biosensing composition comprises at least two different species of microorganisms.

In some embodiments of the biosensing composition, said biosensing composition is formulated for administration to a plant or plurality of plants.

In some embodiments of the biosensing composition, said target molecule is produced by a/said subject.

Probiotics are provided comprising any of the biosensing compositions disclosed herein.

In some embodiments of the probiotic, said probiotic comprises a single species of microorganism.

In some embodiments of the probiotic, said probiotic comprises at least two different species of microorganisms.

In some embodiments of the probiotic, said response factor comprises a postbiotic compound.

In some embodiments of the probiotic, said probiotic comprises a nutritional supplement.

In some embodiments of the probiotic, said probiotic is in the form of a capsule, gel, softgel, paste, tablet, powder, gummy, spray, aerosol, lozenge, effervescent tablet, orodispersible film, topical cream, ointment, suppository, emulsion, transdermal patch, or liquid.

In some embodiments of the probiotic, said probiotic is formulated into a nutritional whole food, drink, sports drink, coffee, tea, water, mineral water, soda, carbonated beverage, oral hydration drink, mineral water, soup, replacement food, nutritional bar, nutritional consumable, gummy, tablet, confectionery, fermented or unfermented milk-based product, yogurt product, milk-based powder, enteral nutritional product, fermented or unfermented cereal-based product, chocolate, milk, yogurt, cheese, ice cream, baby and infant formula, cereal, animal feed, protein shake, protein powder, functional water, juice blend, smoothie, ready-to-drink (RTD) beverage, non-dairy milk, nut or seed butter, energy drink, functional shot, baked good, granola bar, snack bar, alcoholic beverage, frozen food, frozen meal, condiment, pasta, noodles, pet food, or pet treat.

In some embodiments of the probiotic, said probiotic is formulated for administration to a plant or plurality of plants.

In some embodiment of the probiotic, said probiotic is suitable for oral administration to a subject, such as a mammalian subject, such as a human.

In some embodiments of the probiotic, said target molecule is produced by a/said subject.

An engineered yeast cell is provided comprising: a. a molecule responsive element comprising a binding domain that interacts with a target molecule, or a polynucleotide comprising a nucleotide sequence encoding said molecule responsive element; and b. a nucleic acid molecule comprising a polynucleotide comprising a nucleotide sequence encoding a response factor.

In some embodiments of the engineered yeast cell, said molecule responsive element comprises a polypeptide.

In some embodiments of the engineered yeast cell, said polypeptide comprises an amino acid sequence comprising said binding domain.

In some embodiments of the engineered yeast cell, said molecule responsive element comprises a nucleic acid molecule.

In some embodiments of the engineered yeast cell, said polypeptide comprises a protein, such as a fusion protein, a transmembrane protein, a receptor, and/or a functional fragment or variant thereof.

In some embodiments of the engineered yeast cell, said receptor comprises a human glucocorticosteroid (GC) receptor, or a functional fragment or variant thereof having at least 80%, at least 85%, at least 90%, at least 95%, or at least 99% sequence identity to a human glucocorticosteroid (GC) receptor.

In some embodiments of the engineered yeast cell, said human glucocorticosteroid (GC) receptor comprises an amino acid sequence set forth as SEQ ID NO: 1, or a functional fragment or variant thereof having at least 80%, at least 85%, at least 90%, at least 95%, or at least 99% sequence identity to the amino acid sequence set forth as SEQ ID NO: 1; and/or said human glucocorticosteroid (GC) receptor is encoded by a nucleotide sequence set forth as SEQ ID NO: 2, or a variant thereof having at least 80%, at least 85%, at least 90%, at least 95%, or at least 99% sequence identity to the nucleotide sequence set forth as SEQ ID NO: 2.

In some embodiments of the engineered yeast cell, said receptor comprises a G-protein coupled receptor (GPCR) protein, or a functional fragment or variant thereof having at least 80%, at least 85%, at least 90%, at least 95%, or at least 99% sequence identity to a G-protein coupled receptor (GPCR) protein.

In some embodiments of the engineered yeast cell, said G-protein coupled receptor (GPCR) protein is a human G-protein coupled receptor (GPCR) protein.

In some embodiments of the engineered yeast cell, said G-protein coupled receptor (GPCR) protein, or functional fragment or variant thereof, comprises a histamine binding domain, such as a histamine binding ectodomain.

In some embodiments of the engineered yeast cell, said human G-protein coupled receptor (GPCR) protein comprises an amino acid sequence set forth as SEQ ID NO: 3, or a functional fragment or variant thereof having at least 80%, at least 85%, at least 90%, at least 95%, or at least 99% sequence identity to the amino acid sequence set forth as SEQ ID NO: 3; and/or said human G-protein coupled receptor (GPCR) protein is encoded by a nucleotide sequence set forth as SEQ ID NO: 4, or a variant thereof having at least 80%, at least 85%, at least 90%, at least 95%, or at least 99% sequence identity to the nucleotide sequence set forth as SEQ ID NO: 4.

In some embodiments of the engineered yeast cell, said engineered yeast cell further comprises a polypeptide comprising an amino acid sequence encoding a modified yeast GPA1 protein, or a polynucleotide comprising a nucleotide sequence encoding a modified yeast GPA1 protein, wherein said polypeptide: a. comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10; b. comprises an amino acid sequence corresponding with residues 1-467 of SEQ ID NO: 10; c. comprises an amino acid sequence comprising one or more than one amino acid substitution compared to SEQ ID NO: 10, wherein said one or more than one amino acid substitution is selected from the following: K468E, I469Y, G470N, I471L, and I472V; d. comprises an amino acid sequence comprising the following amino acid substitutions: K468E, 1469Y, G470N, 1471L, and I472V, compared to SEQ ID NO: 10; and/or e. comprises an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8; and/or said polynucleotide encoding a yeast GPA1 protein comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

In some embodiments of the engineered yeast cell, said polynucleotide is operably fused to an activation domain.

In some embodiments of the engineered yeast cell, said activation domain comprises a promoter, such as a Ste-12-activated or a human glucocorticosteroid response element (HRE)-activated promoter.

In some embodiments of the engineered yeast cell, said response factor is produced upon binding of STE12 or GC receptor to said promoter.

In some embodiments of the engineered yeast cell, said engineered yeast comprises mutation and/or deletion in at least one of the following genes: STE2, FAR1, SST2, and MTA-A.

In some embodiments of the engineered yeast cell, said nucleotide sequence encoding said molecule responsive element is codon-optimized for expression in a eukaryotic cell, such as a yeast cell.

In some embodiments of the engineered yeast cell, said polynucleotide comprising a nucleotide sequence encoding said molecule responsive element and said polynucleotide encoding a response factor are within a single nucleic acid molecule.

In some embodiments of the engineered yeast cell, said nucleic acid molecule comprises a plasmid.

In some embodiments of the engineered yeast cell, said polynucleotide comprising a nucleotide sequence encoding said molecule responsive element and/or said polynucleotide encoding a response factor are integrated into the genome of said engineered yeast cells.

In some embodiments of the engineered yeast cell, said engineered yeast comprises Saccharomyces, such as Saccharomyces cerevisiae and/or Saccharomyces boulardii.

In some embodiments of the engineered yeast cell, said target molecule comprises a ligand, a protein, metabolite, a hormone, a nutrient, or small molecule, such as cortisol or histamine.

In some embodiments of the engineered yeast cell, said target molecule comprises cortisol or histamine.

In some embodiments of the engineered yeast cell, said engineered yeast cells produce an effective amount of said response factor in response to a threshold concentration of said target molecule.

In some embodiments of the engineered yeast cell, said target molecule is cortisol, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

In some embodiments of the engineered yeast cell, said target molecule is histamine, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

In some embodiments of the engineered yeast cell, said target molecule is produced by a/said subject.

Pharmaceutical compositions are provide comprising any of the biosensing compositions, the probiotics, or the engineered yeast cells disclosed herein, and a pharmaceutically acceptable carrier.

In some embodiments of the pharmaceutical composition, said pharmaceutical composition is suitable for oral administration to a subject, such as a mammalian subject, such as a human.

In some embodiments of the pharmaceutical composition, said target molecule is produced by a/said subject.

Methods of manufacturing a biosensing composition comprising a plurality of engineered yeast cells suitable for detecting, binding, detecting, and/or reducing the levels of a target molecule in a subject are provided, said method comprising the steps of: a) introducing into a yeast cell a nucleic acid molecule comprising a polynucleotide comprising a nucleotide sequence encoding a molecule responsive element comprising a binding domain that interacts with a target molecule; b) introducing into said yeast cell a nucleic acid molecule comprising a polynucleotide comprising a nucleotide sequence encoding a response factor; and c) culturing said engineered yeast cell under conditions sufficient to allow production of a yeast cell culture comprising a plurality of engineered yeast cells comprising said polynucleotide encoding said molecule responsive element and said polynucleotide encoding said response factor.

In some embodiments of the method, the method further comprises the step of: selecting for yeast cells comprising said polynucleotide encoding said molecule responsive element and/or said polynucleotide encoding said response factor.

In some embodiments of the method, at least 50%, at least 55%, at least 60%, at least 65%, at least 70%, at least 75%, at least 80%, at least 85%, at least 90%, at least 95%, or at least 99% of said engineered yeast cells within said plurality comprises said polynucleotide encoding said molecule responsive element and said polynucleotide encoding said response factor.

In some embodiments of the method, introduction of said nucleic acid molecule of step a) and/or step b) occurs simultaneously.

In some embodiments of the method, introduction of said nucleic acid molecule of step a) and/or step b) occurs sequentially.

In some embodiments of the method, said yeast comprises Saccharomyces, such as Saccharomyces cerevisiae and/or Saccharomyces boulardii.

In some embodiments of the method, said molecule responsive element comprises a polypeptide.

In some embodiments of the method, said polypeptide comprises an amino acid sequence comprising said binding domain.

In some embodiments of the method, said molecule responsive element comprises a nucleic acid molecule.

In some embodiments of the method, said polypeptide comprises a protein, such as a fusion protein, a transmembrane protein, a receptor, and/or a functional fragment or variant thereof.

In some embodiments of the method, said receptor comprises a human glucocorticosteroid (GC) receptor, or a functional fragment or variant thereof having at least 80%, at least 85%, at least 90%, at least 95%, or at least 99% sequence identity to a human glucocorticosteroid (GC) receptor.

In some embodiments of the method, said human glucocorticosteroid (GC) receptor comprises an amino acid sequence set forth as SEQ ID NO: 1, or a functional fragment or variant thereof having at least 80%, at least 85%, at least 90%, at least 95%, or at least 99% sequence identity to the amino acid sequence set forth as SEQ ID NO: 1; and/or said human glucocorticosteroid (GC) receptor is encoded by a nucleotide sequence set forth as SEQ ID NO: 2, or a variant thereof having at least 80%, at least 85%, at least 90%, at least 95%, or at least 99% sequence identity to the nucleotide sequence set forth as SEQ ID NO: 2.

In some embodiments of the method, said receptor comprises a G-protein coupled receptor (GPCR) protein, or a functional fragment or variant thereof having at least 80%, at least 85%, at least 90%, at least 95%, or at least 99% sequence identity to a G-protein coupled receptor (GPCR) protein

In some embodiments of the method, said G-protein coupled receptor (GPCR) protein is a human G-protein coupled receptor (GPCR) protein.

In some embodiments of the method, said G-protein coupled receptor (GPCR) fusion protein, or functional fragment or variant thereof, comprises a histamine binding domain, such as a histamine binding ectodomain.

In some embodiments of the method, said human G-protein coupled receptor (GPCR) protein comprises an amino acid sequence set forth as SEQ ID NO: 3, or a functional fragment or variant thereof having at least 80%, at least 85%, at least 90%, at least 95%, or at least 99% sequence identity to the amino acid sequence set forth as SEQ ID NO: 3; and/or said human G-protein coupled receptor (GPCR) protein is encoded by a nucleotide sequence set forth as SEQ ID NO: 4, or a variant thereof having at least 80%, at least 85%, at least 90%, at least 95%, or at least 99% sequence identity to the nucleotide sequence set forth as SEQ ID NO: 4.

79. The method of any one of claims 63-78, further comprising the step of:
introducing into said yeast cell a polynucleotide comprising a nucleotide sequence encoding a polypeptide encoding a modified yeast GPA1 protein,
wherein said polypeptide:
a. comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10;
b. comprises an amino acid sequence corresponding with residues 1-467 of SEQ ID NO: 10;
c. comprises an amino acid sequence comprising one or more than one amino acid substitution compared to SEQ ID NO: 10, wherein said one or more than one amino acid substitution are selected from the following: K468E, I469Y, G470N, I471L, and I472V;
d. comprises an amino acid sequence comprising the following amino acid substitutions: K468E, I469Y, G470N, I471L, and I472V, compared to SEQ ID NO: 10; and/or
e. comprises an amino acid sequence comprising an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8; and/or
wherein said polynucleotide comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

In some embodiments of the method, said polynucleotide sequence encoding said response factor is operably fused to an activation domain.

In some embodiments of the method, said activation domain comprises a promoter, such as a Ste-12-activated or a human glucocorticosteroid response element (HRE)-activated promoter.

In some embodiments of the method, said response factor is produced upon binding of STE12 or GC receptor to said promoter.

In some embodiments of the method, said engineered yeast comprises mutation and/or deletion in at least one of the following genes: STE2, FAR1, SST2, and MTA-A.

In some embodiments of the method, said nucleotide sequence encoding said molecule responsive element is codon-optimized for expression in a eukaryotic cell, such as a yeast cell.

In some embodiments of the method, said nucleotide sequence encoding molecule responsive element and said nucleotide sequence encoding said response factor are comprised within a single nucleic acid molecule.

In some embodiments of the method, said nucleic acid molecule comprises a plasmid.

In some embodiments of the method, said nucleotide sequence encoding said molecule responsive element and/or said nucleotide sequence encoding a response factor are integrated into the genome of said engineered yeast cells.

In some embodiments of the method, said engineered yeast comprises Saccharomyces, such as Saccharomyces cerevisiae and/or Saccharomyces boulardii.

In some embodiments of the method, said target molecule comprises a ligand, a protein, metabolite, a hormone, a nutrient, or small molecule, such as cortisol or histamine.

In some embodiments of the method, said target molecule comprises cortisol or histamine.

In some embodiments of the method, said engineered yeast cells produce an effective amount of said response factor in response to a threshold concentration of said target molecule.

In some embodiments of the method, said target molecule is cortisol, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

In some embodiments of the method, said target molecule is histamine, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

In some embodiments of the method, said response factor comprises a postbiotic compound.

In some embodiments of the method, said biosensing compositions is formulated into the form of a capsule, gel, softgel, paste, tablet, powder, gummy, spray, aerosol, lozenge, effervescent tablet, orodispersible film, topical cream, ointment, suppository, emulsion, transdermal patch, or liquid.

In some embodiments of the method, said biosensing composition is formulated into a nutritional supplement, nutritional whole food, drink, sports drink, coffee, tea, water, mineral water, soda, carbonated beverage, oral hydration drink, mineral water, soup, replacement food, nutritional bar, nutritional consumable, gummy, tablet, confectionery, fermented or unfermented milk-based product, yogurt product, milk-based powder, enteral nutritional product, fermented or unfermented cereal-based product, chocolate, milk, yogurt, cheese, ice cream, baby and infant formula, cereal, animal feed, protein shake, protein powder, functional water, juice blend, smoothie, ready-to-drink (RTD) beverage, non-dairy milk, nut or seed butter, energy drink, functional shot, baked good, granola bar, snack bar, alcoholic beverage, frozen food, frozen meal, condiment, pasta, noodles, pet food, or pet treat.

In some embodiments of the method, said biosensing composition is formulated for administration to a plant or plurality of plants.

Methods of binding, detecting, and/or reducing the levels of a target molecule in a subject in need thereof, said method comprising administering to said subject a therapeutic amount of any of the biosensing compositions, probiotics, or pharmaceutical compositions disclosed herein.

Methods of treating a disease, or disorder, or symptom associated with a disease or disorder, in a subject in need thereof, said method comprising administering to said subject a therapeutic amount of the biosensing compositions, probiotics, or pharmaceutical compositions disclosed herein.

In some embodiments of the methods, said disease or disorder is a stress disorder, an inflammatory disorder, an autoimmune disorder, a disorder associated with microbiome dysbiosis, a hormonal imbalance disorder, an infection, a skin disorder, and/or a gastrointestinal or digestive disorder.

In some embodiments of the methods, said subject comprises elevated cortisol levels or elevated histamine levels.

Methods of maintaining a healthy biological function within a tissue of a subject, said method comprising administering to said subject a therapeutic amount of the biosensing compositions, probiotics, or pharmaceutical compositions disclosed herein.

In some embodiments of the methods, said molecule responsive element interacts with said target molecule.

In some embodiments of the methods, upon interaction of said target molecule and said molecule responsive element, said molecule responsive element promotes increased expression of a gene encoding, or increased production and/or secretion of, the response factor.

In some embodiments of the methods, said increased production and/or secretion of the response factor contributes to reduced levels or reduced activity of said target molecule.

In some embodiments of the methods, said target molecule comprises a ligand, a protein, metabolite, a hormone, a nutrient, or small molecule, such as cortisol or histamine.

In some embodiments of the methods, said target molecule is present on the skin or in the esophagus, nasal tissue, mouth, vaginal tissue, lungs, stomach, small intestines, large intestines, digestive tract, or gastrointestinal tract of said subject.

In some embodiments of the methods, levels of said target molecule are reduced in said subject.

In some embodiments of the methods, said biosensing composition, probiotic, or pharmaceutical composition is administered at least once a day, at least one a week, at least twice a week, at least once a month, at least twice a month, at least once a year, or at least twice a year.

In some embodiments of the method, said target molecule is produced by a/said subject.

### BRIEF DESCRIPTION OF THE DRAWINGS

FIGS. 1A-1B show the dose-dependent detection of cortisol and subsequent response factor production over 6 hours in two engineered S. boulardii strains comprising a wild-type human GC receptor and a hormone response element (HRE)-GFP reporter construct on the same plasmid within the cell. Relative reporter expression (GFP) is shown over time. FIG. 1A shows an engineered S. boulardii strain comprising a polynucleotide (i.e., plasmid) comprising the receptor and reporter in a first orientation. FIG. 1B shows an engineered S. boulardii strain comprising a polynucleotide (i.e., plasmid) comprising the receptor and reporter in a second orientation.

FIG. 2 shows an agarose gel image of PCR reaction products depicting integration of the GC receptor (“Receptor”) polynucleotide and HRE-GFP reporter expression construct (“Reporter”) into the genome of four S. boulardii clones. The polynucleotides were integrated at the HO loci on separate chromosomes. The PCR products are compared to a PCR at the same loci in a wild-type control (Wild-Type).

FIG. 3 shows dose-dependent detection of histamine and response factor production in recombinant S. cerevisiae compared to control S. cerevisiae. Relative GFP levels (GFP(FU)/OD600) are depicted for eight recombinant S. cerevisiae strains. S4-S7 are four S. cerevisiae clones having a histamine receptor polynucleotide and GFP reporter expression construct, in addition to a polynucleotide (SEQ ID NO: 7) encoding a modified GPA1 protein (SEQ ID NO: 8) and gene deletion of STE2, FAR1, SST2, and MAT-A. S1-S3 are three clones of a control strain having a GFP reporter expression construct only. S8 is a control strain having a modified GPA1 protein (SEQ ID NO: 8), lacking a histamine receptor polynucleotide and GFP reporter expression construct.

FIGS. 4A-4D show dose-dependent histamine sensing and response factor production over 16 hours in recombinant S. cerevisiae comprising a histamine receptor polynucleotide and GFP reporter expression construct on two different plasmids. FIG. 4A shows S. cerevisiae having a histamine receptor polynucleotide on a plasmid having a geneticin resistance marker, and a reporter expression construct having a nourseothricin resistance marker. FIG. 4B shows S. cerevisiae having a histamine receptor polynucleotide on a plasmid having a nourseothricin resistance marker, and a reporter expression construct having a geneticin resistance marker. FIG. 4C shows S. cerevisiae having a histamine receptor polynucleotide on a plasmid having a hygromycin resistance marker, and a reporter expression construct having a nourseothricin resistance marker. FIG. 4D shows S. cerevisiae having a histamine receptor polynucleotide on a plasmid having a nourseothricin resistance marker, and a reporter expression construct having a hygromycin resistance marker.

### DETAILED DESCRIPTION OF THE INVENTION

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “of” is used in the inclusive sense of “and/or” and not the exclusive sense of “either/or.”

The term “about” or “approximately” usually means within a value or range close to the stated value or range that accounts for normal variations in measurement, manufacturing, or biological conditions as understood by one skilled in the art. “About” or “approximately,” for example can refer to a value within 5%, or more preferably within 1%, of a given value or range.

The terms “comprises”, “comprising”, “includes”, “including”, “having” and their conjugates mean “including but not limited to”.

As used herein, all polynucleotide sequences written using the nucleic acid standard notation of the International Union of Pure and Applied Chemistry (IUPAC, Biochemistry (1970) Vol. 9:4022-4027); adenine (A), thymine (T), guanine (G), and cytosine (C) are equivalent to the corresponding RNA polynucleotide sequences. Therefore, “T” (Thymine) in all sequences is equivalent to “U” (uracil). For example, the sequence AATAAA in a DNA coding strand would also indicate the corresponding mRNA sequence AAUAAA.

As used herein, the terms “expression cassette” and “expression construct” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors or any other vector known in the art suitable for delivering a polynucleotide to a target cell. The skilled artisan is well aware of the genetic elements that must be present on the vector in order to successfully transform, select and propagate host cells comprising any of the isolated nucleotides or nucleic acid sequences described herein.

As used herein, the term “operably linked” is intended to mean a functional linkage between two or more elements. For example, an operable linkage between a nucleic acid sequence encoding a nuclease as disclosed herein and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

A “culture media” or “growth media”, used interchangeably herein, refers to a substance that provides essential nutrients for the growth of microorganisms. Culture media is used as a source of energy for a microorganism, such bacteria or yeast. For instance, and without limitation, culture media may be used to promote the growth of one or more microorganisms (e.g., a plurality of microbial cells). Culture media may include a solid, liquid, or gel substance. In a non-limiting exemplary embodiment, culture media may include a nutrient broth having meat extracts and peptone water. Nutrient broths may further include glucose, yeast, polysorbates, and derivatives thereof. Culture media may further include synthetic media wherein the synthetic media is a defined medium having known chemicals and a lack of yeast, plant, or animal tissue. Culture media can be specifically formulated to achieve a desired impact on a microorganism, such as a physiological impact, during the growth cycle. For example, it would be understood that the gene expression profile, and corresponding metabolome profile, may be impacted by culture media. As such, the culture media can be specifically formulated to comprise components that closely mimic conditions (e.g., chemical conditions) encountered by a microorganism in or in a target tissue or target organ following administration. Without wishing to be bound by any theory, it is possible that growing a microorganism under conditions that simulate the target organ and/or tissue encountered following administration while enhance the efficacy of the microorganism following administration. The efficacy of the microorganism can refer to the ability of a microorganism to achieve any of the outcomes disclosed herein. In some embodiments, the culture medium is formulated to mimic the gastrointestinal or digestive tract, or region thereof. In some embodiments, the culture medium is formulated to mimic the respiratory tract, or a region thereof (e.g., airways or lungs). In some embodiments, the culture media is formulated to mimic the environment of the skin. In such instances, the efficacy of a microorganism bind, detect, and/or reduce the levels of a target molecule, reduce a symptom of a disease, produce a response factor, and/or maintain healthy biological function in a tissue is improved following administration.

The term “molecule responsive element” refers to a molecule that is membrane-bound or located within the cell of a microorganism that interacts with a target molecule and, modifies the expression, activity, or amount of a response factor within the cell. Following interaction with the target molecule, the molecule responsive element can initiate expression of a gene encoding, or production and/or secretion of, the response factor within the cell. A molecule responsive element can be a transmembrane protein that is bound to the cell that interacts with a molecule located outside of a cell, and subsequently transmits a signal inside the cell via, for example, signal transduction. The molecule responsive element can also be a molecule that is located within the cell that interacts with a target molecule that enters the cell. A molecule responsive element can interact with and may bind to a target molecule and can be involved in directly activating expression of a gene encoding a response factor by interacting with an activation domain, such as a promoter. In such instances, the molecule responsive element can detect a target molecule outside of the cell nucleus, and upon interacting with the target molecule, signal to the cell nucleus to activate gene expression. The molecule responsive element can also indirectly activate expression of response factor gene by initiating a signaling cascade within the cell. In a particular embodiment, the molecule responsive element is a polypeptide or a polynucleotide comprising a binding domain that is specific for the target molecule (i.e., specifically interacts with or binds to the target molecule). The terms “molecule responsive element” and “molecule response element” are used interchangeably herein.

A “target molecule” refers to any molecule that interacts with a molecule responsive element disclosed herein. The target molecule can be produced by the subject or otherwise present in the environment of the cell.

The term “response factor” refers to a molecule that is produced and/or secreted from a cell upon interaction of a target molecule with a molecule responsive element in the cell. The response factor is specific to the target molecule being detected. For example, the response factor is a molecule that ultimately participates in reducing the levels of the target molecule or disrupts function of the target molecule. The response factor can act directly on the target molecule, or can act indirectly through intermediates for the desired modulation of the target molecule. In some instances, the response factor is a molecule that reduces cortisol levels and/or activity. The response factor can be a molecule that reduces histamine levels and/or activity. The response factor will be secreted from the engineered cell, and as such can act on a target molecule within a tissue or surrounding environment.

A “transmembrane protein” refers to a protein that spans the entirety of a cell membrane, with parts of it on both the inside and outside of the cell. Transmembrane proteins are a type of integral membrane protein, which are proteins that are permanently attached to the cell membrane. A transmembrane protein can transmit signals from outside of the cell to inside of the cell through the use of signal transduction pathways which may involve additional cellular components. For instance, a transmembrane protein may interact with or bind to a molecule located outside of the cell and initiation a signaling cascade that impacts gene expression within the cell.

The term “activation domain” refers to a portion of a polynucleotide sequence that is involved in controlling expression of a gene located within the polynucleotide sequence. An activation domain can be located 5′ or upstream of the gene encoding polynucleotide sequence. Often, the activation domain interacts with an activator protein, such as a transcription factor, to initiation transcription (i.e., expression) of a downstream gene. An activator protein may bind to an activation domain to initiate transcription. An activation domain can comprise a polynucleotide sequence that is specific for certain transcription factors. In some instances, the activation domain specifically interacts with a transcription factor. In a particular embodiment, the activation domain interacts with STE12. In another embodiment, the activation domain interacts with a GC receptor.

The term “codon-optimized” refers to a process used to improve gene expression and increase the translational efficiency of a gene of interest by accommodating codon bias of the host organism. A polynucleotide sequence can be codon-optimized for expression in yeast. In some instances, a polynucleotide sequence of a human gene is codon-optimized for expression in yeast. Modifying nucleic acid sequence codons relies upon the fact that in eukaryotic cells there are multiple three base codons that encode the same amino acid, wherein the third position in the codon can be selected from multiple nucleotides, i.e., can be “wobbled”. As used herein, the use of the term “polynucleotide”, “DNA”, or “nucleic acid” is not intended to limit the disclosure to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides described herein also encompass all forms of sequences including, but not limited to, single-stranded forms, double-stranded forms, hairpins, stem-and-loop structures, and the like.

The term “inoculation” as used herein refers to the introduction of a microorganism (or plurality of microbial cells) to a substrate or composition. This may include placing the substrate in the presence of one or more microorganisms. Inoculation may include placing the substrate within a liquid containing one or more microorganisms or placing the microorganisms within a composition containing a substrate. For example, and without limitation, inoculation may include placing a microbial cell, or plurality of microbial cells, into a liquid solution containing a substrate.

The term “effective amount” or “therapeutic amount”, used interchangeably herein, refers to a quantity of a biosensing composition comprising a plurality of engineered yeast cells comprising a molecule responsive element comprising a binding domain that interacts with a target molecule, or a polynucleotide comprising a nucleotide sequence encoding the molecule responsive element, and a nucleic acid molecule comprising a polynucleotide encoding a response factor disclosed herein to perform any of the methods and/or uses described herein. For instance, an effective amount of the biosensing composition, or probiotic or therapeutic composition comprising a biosensing composition, can bind, detect, and/or reduce the levels of a target molecule, reduce a symptom associated with a disease or disorder, and/or maintain a healthy biological function in a tissue of a subject. Appropriate controls for the methods are described herein. An effective amount of the biosensing composition, or probiotic or pharmaceutical composition comprising the biosensing composition, can be determined based on the desired outcome. The quantity to be administered to a subject, both according to number of treatments and effective amount, depends on the subject to be treated, the state of the subject, the environmental conditions of the subject, and the result desired. Precise amounts of the biosensing also depend on the judgment of the practitioner and can be unique to a subject and/or individual.

The term “engineered” or “recombinant” in the context of a microorganism refers to a microorganism, or population of the microorganism, that has been modified (by selection and/or transformation) to have one or more additional traits of interest. Recombinant microorganism cells (e.g., strains) can be made through genetic engineering techniques and such engineered or recombinant strains grown to produce a population of engineered cells. An engineered strain can be produced by introducing polynucleotides into the host cell by transformation or by otherwise altering the native chromosome sequence, including but not limited to, gene editing approaches. Methods for transforming microorganisms are known in the art. See, generally, Hanahan, D. (1983) Studies on transformation of Escherichia coli with plasmids J. Mol. Biol. 166, 557-77; Seidman, C. E. (1994) In: Current Protocols in Molecular Biology, Ausubel, F. M. et al. eds., John Wiley and Sons, NY; Choi et al. (2006) J. Microbiol. Methods 64:391-397; Wang et al. 2010. J. Chem. Technol. Biotechnol. 85:775-778. Transformation may occur by natural uptake of naked DNA by competent cells from their environment in the laboratory. Alternatively, cells can be made competent by exposure to divalent cations under cold conditions, by electroporation, by exposure to polyethylene glycol, by treatment with fibrous nanoparticles, or other methods well known in the art. An engineered microorganism can comprise a molecule, such as a polynucleotide or polypeptide, that is not present in the cell in nature. In some instances, an engineered strain produces a larger amount of a particular molecule, wherein said molecule can be a molecule that is produced by the cell in nature, or is a heterologous molecule not found in the cell in nature. In some instances, an engineered microorganism comprises a recombinant molecule.

The term “GC receptor polynucleotide” refers to a polynucleotide encoding a GC receptor polypeptide, or functional fragment or variant thereof. GC receptor polypeptide sequences are known in the art. In some instances, the GC receptor polypeptide can comprise a human GC receptor polypeptide.

The term “human histamine receptor polynucleotide” refers to a polynucleotide encoding a human histamine receptor polypeptide, or a functional fragment or variant thereof. Human histamine receptor polypeptide sequences are known in the art.

The term “HRE expression construct”, “HRE component”, and “HRE module”, used interchangeably herein, refers to a polynucleotide comprising a hormone response element (HRE) nucleotide sequence operably linked to a polynucleotide encoding a response factor. Thus, production of the response factor can be regulated by the HRE polynucleotide. One having ordinary skill in the art would readily understand a HRE as a short, specific DNA nucleotide sequence that serves as a binding site for a glucocorticoid receptor (GR) such as GC receptor that has been activated (e.g., undergoes a conformational change) upon interaction with cortisol. HREs are usually found in the promoter or enhancer regions of glucocorticoid-responsive genes, a position that allows receptors to influence transcription initiation. For instance, interaction of a GC reporter with an HRE polynucleotide can result in the recruitment of coactivators or corepressors and interaction with RNA polymerase II machinery, thereby upregulating or downregulating gene expression. Operably linking a polynucleotide encoding a gene can serve as a means of regulating gene expression via HRE. The HRE expression construct can comprise a nucleotide sequence encoding any gene product useful for being driven by the HRE. The “HRE-GFP expression construct”, “HRE-GFP component”, or “HRE-GFP module”, described interchangeably herein, refers to a HRE polynucleotide operably linked to a polynucleotide encoding a GFP.

As used herein, the term “postbiotic compound” refers to inanimate microorganisms and/or their components. The term “inanimate” when used in reference to microorganisms of a postbiotic agent means lifeless, rather than ‘inactive, as this latter term might suggest an inert material. ‘Inanimate’ simply captures the fact that live microorganisms were present but have now been killed, without implying a loss of function. A postbiotic agent can include non-viable agents capable of being produced by a microorganism such as bacteria (e.g., prebiotic bacteria or lactic acid bacteria) or yeast or byproducts thereof. A postbiotic agent can be any substance derived from a microorganism, including compounds secreted by live bacteria (e.g., metabolic byproducts) or released after bacterial lysis, such as metabolites, enzymes, peptides, teichoic acids, peptidoglycan-derived muropeptides, polysaccharides, cell surface proteins, or organic acids, as described in Salminen S, Collado M C, Endo A, Hill C, Lebeer S, Quigley E M M, Sanders M E, Shamir R, Swann J R, Szajewska H, Vinderola G. The International Scientific Association of Probiotics and Prebiotics (ISAPP) consensus statement on the definition and scope of postbiotics. Nat Rev Gastroenterol Hepatol. 2021 September; 18(9):649-667, the contents of which are incorporated herein in their entirety. Any postbiotic agent can be present in the postbiotic composition. Substances derived from a microorganism can include fragments or constituents of a microbial cell that is nonviable and intact, or ruptured and lysed. For example, the postbiotic may be one or more cell wall fragments, exopolysaccharides, membrane vesicles, or genetic material that is released from an intact microbial cell following cell lysis. The terms “postbiotic”, “postbiotic agent”, and “postbiotic compound” are used interchangeably herein. Postbiotics possess different functional properties. These properties can positively affect the microbiota homeostasis and/or the metabolic and signaling pathways in an individual, thus affecting specific physiological, immunological, neuro-hormone, biological, regulatory, or metabolic reactions.

The term “fragment” refers to a portion of a polynucleotide or polypeptide sequence. “Fragments” or “biologically active portions” include polypeptides comprising a sufficient number of contiguous nucleotides to retain the biological activity (e.g., binding a target molecule, activating expression of a response factor gene). Fragments of polypeptides include those that are shorter than the full-length sequences due to the use of an alternate downstream start site. A biologically active portion of a molecule responsive element can be a polypeptide that comprises, for example, 10, 20, 30, 40, 50, 60, 70, 80, 90, 100, 110, or more contiguous amino acid residues of any polypeptide sequence disclosed herein. Such biologically active portions can be prepared by recombinant techniques and evaluated for activity.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein. Disclosed herein are polynucleotides comprising a nucleotide sequence encoding a molecule responsive element. A polynucleotide can encode any molecule responsive element disclosed herein. A polynucleotide can comprise a variant nucleotide sequence encoding any molecule responsive element disclosed herein. In some instances, the polynucleotide comprises a nucleotide sequence encoding a GC receptor. In some instances, the polynucleotide comprises a nucleotide sequence encoding a human GC receptor. The polynucleotide can comprise a variant sequence having at least 80%, at least 85%, at least 90%, at least 95%, or at least 99% sequence identity to the nucleotide sequence encoding a GC receptor. The polynucleotide can comprise a variant sequence having at least 80%, at least 85%, at least 90%, at least 95%, or at least 99% sequence identity to the nucleotide sequence encoding a human GC receptor. In some instances, the polynucleotide comprises a nucleotide sequence encoding a histamine receptor. In some instances, the polynucleotide comprises a nucleotide sequence encoding a human histamine receptor. The polynucleotide can comprise a variant sequence having at least 80%, at least 85%, at least 90%, at least 95%, or at least 99% sequence identity to the nucleotide sequence encoding a histamine receptor. The polynucleotide can comprise a variant sequence having at least 80%, at least 85%, at least 90%, at least 95%, or at least 99% sequence identity to the nucleotide sequence encoding a human histamine receptor.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

A “plant” refers to a whole plant, any part thereof, or a cell or tissue culture derived from a plant, comprising any of: whole plants, plant components or organs (e.g., leaves, stems, roots, embryos, pollen, ovules, seeds, leaves, flowers, branches, fruit, pulp, juice, kernels, ears, cobs, husks, stalks, root tips, anthers, bark, etc.), plant tissues, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the plants are also included within the scope of the invention. Also included are plants in which a genetic alteration, such as a mutation, has been effected as to a gene of interest, or within the genomic sequence of the plant, or a plant which is descended from a plant or plant cell so altered and which comprises the alteration in the genomic sequence. The terms “plant”, “plants”, and “plant parts” are used interchangeably herein.

As used herein with respect to a parameter, the term “decreased” or “decreasing” or “decrease” or “reduced” or “reducing” or “reduce” or “lower” or “loss” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “decreased”, “reduced”, and the like encompass both a partial reduction and a complete reduction compared to a control.

As used herein with respect to a parameter, the term “increased” or “increasing” or “increase” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, 120%, 150%, 200%, 300%, 400%, 500%, or more) positive change in the parameter from a comparison control, e.g., an established normal or reference level of the parameter, or an established standard control. Accordingly, the terms “increased”, “increase”, and the like encompass both a partial reduction and a significant increase compared to a control.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

A “microorganism” will be understood to be a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers.

A “nutritional supplement”, for the purposes of this disclosure, is an edible substance intended to provide nutritional value to a subject when consumed.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

All publications, patent applications, patents, and other references mentioned herein are incorporated by reference herein in their entirety.

### II. Overview

The present disclosure relates to compositions and methods for detecting and responding to target molecules. The compositions disclosed herein can find use in biosensing specific substances and conditions in or on a subject, such as a human or animal subject. In particular, the compositions and methods can be used for detecting and reacting to changes in specific substances and conditions using live organisms, such as probiotics. The compositions and methods can involve live microorganisms to detect, bind, and/or reduce levels of a target molecule in or on a tissue of a subject/animal. In some instances, the microorganisms can be yeast. The compositions and methods disclosed herein can use probiotics and/or yeast cells that have been engineered. In some instances, the compositions and methods use yeast that are engineered to re-purpose natural biosensing components and pathways found in nature for use in a new and beneficial context. The involvement of a plurality of molecular components functioning within biological biosensing pathways would be readily understood by a person having skill in the art. In a specific embodiment, yeast is engineered to encode and/or comprise a heterologous molecule, or signaling pathway comprising a plurality of molecules, found naturally in a different organism, such as a human. In some embodiments, the engineered yeast comprise molecular components involved in biosensing that are naturally found in a different species (i.e., naturally found in a species other than yeast).

Natural biosensing molecules and signaling pathways can be re-purposed in order to link the production of a response molecule to a target molecule, wherein said response molecule is not typically associated with the target molecule in nature. In some embodiments, the molecular components (e.g., target responsive molecule, response factor) are designed to specifically link the sensing of a target molecule or environmental condition to the production of a specific response factor. In some embodiments, the response factor produced is not naturally produced in yeast. In some instances, the response factor is not naturally produced in an organism in response to the target molecule. That is, it may be possible that the target molecule disclosed herein has not been demonstrated to drive production of the response factor within an organism in nature. In a specific embodiment, yeast can be engineered to produce a response factor following interaction of cortisol that is not known to be produced in response to cortisol in nature. As such, the microorganism disclosed herein, such as yeast, can be engineered to comprise and utilize heterologous sensing and signaling components in order to bind and detect target molecules and/or environmental conditions and produce any response factor disclosed herein.

Live organisms are particularly effective for biosensing in or on a subject, such as on the skin or in the gastrointestinal tract. One aspect of the engineered microorganisms disclosed herein and methods of using the same involves detecting and binding to substances. The present disclosure also includes the production and/or secretion of a response factor. The response factor can be a molecule and/or metabolite that is produced and/or secreted in response to the presence of a target molecule. Importantly, the production and/or secretion of the response factor occurs only in response to the detection and/or interaction of a molecule responsive element with a target molecule. The amount of the response factor produced can be controlled, such that only the appropriate amount is produced and/or secreted. An appropriate amount when used in reference to a response factor is intended to mean an amount that is capable of achieving a desired outcome upon detection and/or interaction with a specific target molecule, as described further herein. For instance, a desired outcome can be, for example, inactivation and/or degradation of a target molecule. Thus, an appropriate amount of the response factor can be determined as a function of the amount of a given target molecule.

In one particular aspect, the compositions and methods disclosed herein are particularly effective at detecting and reacting to changes in the levels (e.g., concentration) of a target molecule. For instance, the engineered microorganisms find use in monitoring the levels of a target molecule. To monitor the levels of a target molecule, the engineered microorganism remains present in or on a subject for a period of time following administration. For instance, the engineered microorganism will reside in the subject (i.e., colonize the subject) in the desired tissue. The engineered microorganism can be present on the skin or within the gastrointestinal tract of a subject for a period of time following administration. Any period of time is envisaged within the scope of the present disclosure for an amount of time in which the engineered microorganisms are present within or on a tissue of the subject. Within or on the tissue of a subject, the engineered microorganisms will produce and/or secrete a response factor upon interaction of a molecule responsive element with a target molecule. Importantly, the microorganisms can be engineered such that a response factor is only produced and/or secreted when the target molecule is detected at, or above, a threshold concentration. For instance, some levels of a target molecule may not be sufficient to promote the production and/or secretion of a response factor by the engineered microorganism. In such instances, a specific amount of the target molecule (i.e., threshold level) must be present to instigate a response from the engineered cell. In some embodiments, the engineered cell will only generate a response factor in response to elevated levels of the target molecule. Levels of a target molecule are “elevated” as described herein when they are increased above the levels typically found in a tissue, such as levels that are required to perform normal physiological roles within a tissue. The amount and/or levels of a target molecule can be defined as a concentration of the target molecule within or on a particular tissue. The threshold level of the target molecule necessary for response can be modulated to control the amount and activity of the response factor. Upon the detection of a specific amount of the target molecule, the engineered microorganism can produce and/or secrete only an appropriate amount of the response factor. Once the target molecule is present at levels below the target threshold, the engineered microorganism can stop production and/or secretion of the response factor.

The compositions and methods disclosed herein are particularly effective at providing targeted and personalized nutrient and/or health interventions. The compositions and methods can reduce the levels of a target molecule associated with a condition and/or disorder in a subject to maintain and/or restore healthy tissue in a subject. The compositions and methods provide an intervention with maximized efficacy, as the response factor is produced only when a target molecule is present at or above a desired concentration threshold (i.e., threshold level). For instance, following administration, the engineered microorganism, which can transiently reside in or persistently colonize a specific tissue of the subject, will produce and/or secrete a response factor when a target molecule is present at or above a specific concentration threshold. This aspect of the disclosed compositions and methods serves to minimize the potential side effects that may be associated with known health interventions. In particular, the amount of the response factor that is produced, secreted and, thus, delivered to a specific tissue in a subject is tightly controlled in the location (e.g., tissue), timing (i.e., only when a specific target molecule is present at a specific level), and concentration (i.e., an appropriate amount of the response factor is produced and/or secreted).

The engineered yeast disclosed herein can comprise a molecule responsive element comprising a binding domain capable of interacting with a target molecule. In certain embodiments, the binding domain is located on or outside of a cell surface. Upon interaction with a target molecule, the molecule responsive element, which can further comprise a signal transduction element, can subsequently modify the expression, activity, or amount of a target-specific effector response molecule (i.e., response factor). In some embodiments, the response molecule can be released from the cells into the environment. In certain embodiments, the release of the response factor increases the amount and/or concentration of the response factor in the environment and can promote the reduction in levels or functionality of the target molecule. In certain embodiments, the response factor is produced and released when the target molecule is present at a given level and/or concentration threshold. The response factor may promoter the reduction in levels or functionality of the target molecule directly or indirectly. For instances, the response factor may promote the reduction in levels or functionality of an intermediate molecule (i.e., a molecule that is not the target molecule). In such instances, the intermediate molecule may directly impact the levels or functionality of the target molecule. In some instances, multiple molecules are involved in regulating the levels and/or functionality of the target molecule. As such, the response factor may act on any molecule that directly and/or indirectly promotes the reduction in levels or functionality of a target molecule. The engineered yeast can be designed to detect and interact with a target molecule that is present at levels above a given threshold. Levels of a target molecule at or above a specific threshold may be indicative of a state of disease and/or dysbiosis within a host tissue. For instance, elevated levels of a target molecule may be indicative of a stress disorder, an inflammatory disorder, an autoimmune disorder, a disorder associated with microbiome dysbiosis, a hormonal imbalance disorder, an infection, a skin disorder, and/or a gastrointestinal or digestive disorder. The engineered yeast can detect elevated levels of a target molecule in or on any tissue of a subject, and subsequently produce a response factor. In some instances, a response factor may function to treat and/or reduce a sign or symptom associated with a disease or disorder or dysbiosis. A response factor may act on any molecule that directly and/or indirectly promotes a sign or symptom associated with a disease or disorder or dysbiosis to treat and/or reduce a sign or symptom associated with a disease or disorder or dysbiosis.

In some instances, a response factor promotes the reduction in levels or functionality of a molecule that promotes a sign or symptom associated with a disease or disorder or dysbiosis. In such instances, the molecule may be the target molecule. In some instances, the molecule may be an intermediate molecule that is not the target molecule.

An engineered yeast composition can comprise a probiotic comprising at least one live yeast cell that can be orally administered to a subject. Upon oral administration to a subject, an engineered yeast can monitor and detect a target molecule and subsequently produce an effector response molecule in vivo in or on a subject. An engineered yeast can colonize the gastrointestinal tract of the subject, providing prolonged in vivo target molecule monitoring. An engineered yeast is particularly effective at detecting the presence and concentration of a target molecule in the gastrointestinal tract in a subject. As such, the methods of the present disclosure can comprise monitoring and detection of the presence of a target molecule without the use of instrumentation or direct measurement via sample collection.

There is a need in the art for enhanced compositions and methods for monitoring and detecting the levels and/or concentrations of target molecules in and on a subject, and subsequently administering an effective therapeutic to lower the levels of the target molecules.

### III. Biosensing Compositions

Biosensing compositions comprising a plurality of microorganisms that are engineered to interact with a target molecule and produce a response factor are disclosed. Any microorganism can be engineered to interact with a target molecule and produce a response factor. In a specific embodiment, the microorganisms comprise yeast cells. In particular, the yeast comprises Saccharomyces, such as Saccharomyces cerevisiae and/or Saccharomyces boulardii. The biosensing compositions described herein are useful in applications when binding, detecting, and/or reducing the levels and/or activity of a target molecule can improve the health of a subject, or reduce a sign or symptom of a disorder in a subject. The target molecule may be present at elevated levels in any tissue of a subject, for any period of time. For instance, the elevated levels can be chronic or transient. In some instances, the elevated levels of the target molecule are non-pathological (e.g., a non-pathological increase in levels). The response factor is produced in engineered yeast cells when the target molecule exceeds a concentration threshold. The response factor can be produced and secreted from the yeast cells for as long as the target molecule is present and detected above a concentration threshold, for any period of time, until the levels of the target molecule are reduced below the threshold.

The biosensing compositions disclosed herein can comprise a plurality of engineered yeast cells comprising a molecule responsive element comprising a polypeptide comprising a binding domain that interacts with a target molecule, or a polynucleotide comprising a nucleotide sequence encoding said polypeptide, and a nucleic acid molecule comprising a polynucleotide encoding a response factor.

The present disclosure provides compositions and methods comprising an engineered yeast cell capable of binding and/or interacting with a target molecule. Any genus, species, strain, and/or subspecies of yeast is envisaged as capable of being engineered to interact with a target molecule and produce a response factor as described herein. Also disclosed herein is an engineered yeast cell, and methods of making the same, capable of detecting a target molecule in vivo in or on a subject. In particular, the yeast cell of the present disclosure comprises a molecule responsive element that interacts with a target molecule and subsequently activates gene expression, or production of a response factor, capable of modulation of the target molecule. The yeast cell disclosed herein may comprise a molecule responsive element that interacts with a target molecule and subsequently activates host gene expression to instigate production of a response factor by host cells. Moreover, yeast cell disclosed herein may comprise a molecule responsive element that interacts with a target molecule and subsequently activates gene expression of other organisms or cells in the yeast environment to instigate production of a response factor by those other organisms or cells.

The yeast cell can be engineered to comprise one or more genetic (e.g., polynucleotide) or molecular (e.g., molecule responsive elements, including receptors, binding domains, or polypeptides comprising the same) components disclosed herein. The term “engineered”, as used herein, refers to a yeast cell comprising one or more genetic or molecular components that is introduced directly into the yeast cell or its parent yeast cell, and acquired and maintained through replication of said parent yeast cell, by a method selected from the group consisting of recombinant DNA techniques (e.g., Reiterative Recombination and CRISPR), natural genetic events, conjugation, and a combination thereof. A person of skill in the art would understand a yeast cell to be a eukaryotic, single-celled microorganism belonging to the fungus kingdom. In certain embodiments, the term yeast cell as used herein can describe a fungal species, or fungus. Non-limiting examples of yeast cells include Saccharomyces cerevisiae, S. cerevisiae boulardii, Pichia pastoris and Schizosaccharomyces pombe. In certain embodiments, the yeast cell is Saccharomyces cerevisiae. Additional non-limiting examples of fungal cells, that as disclosed herein can be included as yeast cell include Candida albicans, Paracoccidioides brasiliensis, Fusarium graminearum, Magnaporthe oryzae, and Botrytis cinerea. Any species of yeast capable of being engineered to for use in the methods disclosed herein is envisaged within the scope of the present disclosure. In some embodiments, the yeast can be Saccharomyces cerevisiae or S. cerevisiae boulardii). The yeast can be a probiotic yeast. In some embodiments, the engineered organism is a filamentous fungi, such as Aspergillus niger, Aspergillus oryzae, Penicillium roqueforti, Penicillium camemberti, Rhizopus oryzae, Mucor circinelloides, Trichoderma reesei, Monascus purpureus, and/or Fusarium venenatum. Any filamentous fungi used in food fermentation can be used.

Disclosed herein are compositions and methods for in vivo detection in a subject the presence of a target molecule, or a fragment thereof, for example, but not limited to, a molecule associated or correlated with a human disease, or a sign or symptom associated with a human disease, such as a biomarker associated with a disease or ailment (e.g., a pathogenic agent, a hormone, a dietary molecule, or an inflammatory molecule). The target molecule may include, but is not limited to, any molecule in or on the body that influences or reflects normal or abnormal physiological processes. Examples of target molecules include hormones, dietary components, metabolites, environmental substances, pathogenic agents, inflammatory molecules, or molecules involved in maintaining homeostasis and overall health. The detection and modulation of such target molecules can support or enhance normal bodily structures or functions in healthy individuals and may also aid in the management or treatment of diseases or disorders in affected individuals. The target molecule may be elevated in a tissue in a subject. Elevated levels of the target molecule may or may not be associated with a sign, symptom, or pathology. In some instances, the target molecule is elevated only for a period of time. In some instances, the target molecule is elevated for an extended period of time. A fragment of the target molecule can include a portion generated by proteolysis or degradation, wherein the fragment is capable of interacting with a molecule responsive element of the present disclosure. The compositions and methods utilize at least one yeast cell engineered to comprise a molecule responsive element that can interact with a target molecule. The molecule responsive element can comprise a polypeptide or a nucleic acid molecule. The polypeptide can comprise a fusion protein, receptor, transmembrane protein, or any known polypeptide capable of being engineered to bind a target molecule and initiate a signaling cascade within the cell to increase gene expression within the cell. For instance, transcription factors can be good candidates for engineering as a molecule responsive element due to their ability to activate gene expression. A transcription factor that is engineered to interact with a target molecule will maintain the ability to activate gene expression. The molecule responsive elements will comprise a binding domain that interacts with the target molecule. Interaction with a target molecule can trigger a signaling cascade that results in the production of a response factor in at least one engineered yeast cell. The response factor can be encoded by a polynucleotide comprising a polynucleotide sequence. The polynucleotide can be comprised within a gene cassette. The response factor, or polynucleotide encoding the response factor, can be coupled to the molecule responsive element. The term “coupled” as used in relation to the molecule responsive element and the response factor refers to the production of the response factor, or activation of expression of the polynucleotide encoding the response factor, subsequent to interaction of the molecule responsive element to the target molecule. In some embodiments, the response factor is coupled to the molecule responsive element through a signaling cascade. The signaling cascade can be any molecular signaling cascade within the engineered yeast capable of transmitting the signal of target molecule binding resulting in production of the response factor. In some embodiments, when the molecule responsive element comprises a G-protein coupled receptor, the signaling cascade comprises a mitogen-activated protein kinase (MAPK) phosphorylation cascade. Exemplary signaling cascades are known in the art.

The target molecule can be any molecule that is present at elevated levels, for any period of time, within at least one tissue in a subject. The tissue, for example, can include the skin or in the esophagus, nasal tissue, mouth, vaginal tissue, lungs, stomach, small intestines, large intestines, digestive tract, or gastrointestinal tract of said subject. The target molecule is a molecule that is produced by the subject. In some instances, the target molecule is produced at levels that are elevated within a specific tissue compared to a healthy individual. The target molecule can be produced in the tissue in which levels are elevated. Alternatively, the target molecule may be produced in a different tissue and localized to other tissues. Target molecules may or may not be associated with a disease, disorder, and/or ailment. The target molecule can be associated with any known disorder in any tissue of a subject. In some instances, the target molecule is associated with a stress disorder, an inflammatory disorder, an autoimmune disorder, a disorder associated with microbiome dysbiosis, a hormonal imbalance disorder, an infection, a skin disorder, and/or a gastrointestinal or digestive disorder.

Target molecules associated with human disease or ailments can include, but are not limited to, infectious disease agents, oncological disease agents, neurodegenerative disease agents, kidney disease agents, cardiovascular disease agents, gastrointestinal disease agents, and allergen and toxin agents.

Infectious disease agents include, but are not limited to, fungal pathogens, bacterial pathogens, viral pathogens, and protozoan pathogens, as well as toxins produced by same. Non-limiting examples of fungal pathogens include C. albicans, C. glabrata, P. brasiliensis, L. elongisporous, P. rubens, C. guillermondi, C. tropicalis, C. parapsilosis, C. lusitaniae, S. scheckii, and Candida krusei.

Non-limiting examples of bacterial pathogens include Vibrio cholerae, Staphylococcus aureus and Methicillin-resistant Staphylococcus aureus (MRSA) strains, Bacillus subtilis, Streptococcus pneumonia, Group B Streptococcus, Salmonella sp., Listeria monocytogenes, Chlamydia trachomatis, Neisseria gonorrhoeae, Clostridium difficile, Yersinia enterocolitica, Legionella sp., Mycobacterium tuberculosis, Klebsiella pneumoniae, Klebsiella oxytoca, Serratia marcescens, Neisseria meningitis, Streptococcus pneumoniae, Pseudomonas aeruginosa, Streptococcus pyogenes, botulinum toxin of Clostridium botulinum, Shigella/Enteroinvasive E. coli, Shiga toxin from the Shiga toxin-producing Escherichia coli (STEC), and Verotoxin derived from Shigella dysenteriae. Target molecules that are indicative of the presence of bacterial pathogens include, but are not limited to, quorum sensing small molecules such as the Vibrio Cholera CAI-1, inter-species bacterial quorum sensing AL-2, or components of the bacterial LPS.

Non-limiting examples of viral pathogens include Ebola virus, HPV, HIV, influenza, Hepatitis C Virus, Hepatitis B Virus. Cytomegalovirus (CMV), Epstein-Barr virus (EBV), Respiratory syncytial virus (RSV), Norovirus, Sapovirus, and measles virus. Target molecules that are indicative of the presence of viral pathogens include, but are not limited to, capsid protein or peptides, and other viral particles.

Non-limiting examples of protozoan pathogens include Trichomonas vaginalis, Cryptosporidium, Cyclospora cayetanensis, Giardia lamblia, and biomarkers for Amoebiasis derived from Entamoeba histolytica such as E. histolytica ADP-forming acetyl-CoA synthetase (EhACS) or related peptides [Huat (2014)], Leishmaniasis biomarkers such as the amastin signature peptide [Rafati (2006)].

Oncological disease molecules include, but are not limited to, lung, breast, colorectum, prostate, stomach, liver, kidney or cervix cancer, leukemia, Kaposi sarcoma, Testis, Ovary, thyroid, and other cancer peptide biomarkers unique for certain cancer types, which can be identified by mass spectrometry.

Neurodegenerative disease molecules include, but are not limited to, peptide biomarkers indicated in Alzheimer's, [notably fungal biomarker for Alzheimer's were recently suggested in Pisa (2015)], the protein DJ-1 or peptides thereof as biomarkers for Parkinson disease, and biomarkers for prion disease such as proteins or peptides of the 14-3-3 family in cerebrospinal fluid for detection of Creutzfeldt-Jakob disease [Van Everbroeck (2005) and Huzarewich (2010)].

Allergen and toxin agents include, but are not limited to, peptide derived from immunogenic wheat peptide (e.g., gluten), and carcinogen aflatoxin B1 derived from the fungi A. flavus.

Kidney disease molecules include, but are not limited to, proteins and peptides identified as urinary biomarkers for kidney disease, such as 02-microglobulin, and differential patterns of peptides in type 2 diabetes.

Cardiovascular disease molecules include, but are not limited to, proteins and peptides indicative for atherothrombosis or risk markers for stroke. Markers for primary cardiovascular events include peptides derived from C-reactive protein, fibrinogen, cholesterol, apolipoprotein B, high density lipoprotein, and small molecules like vitamin D. Markers for secondary cardiovascular events include peptides derived from cardiac troponins I and T, C-reactive protein, serum creatinine, and cystatin C. Risk markers for primary stroke, include peptides derived from fibrinogen and serum uric acid [Van Holten et al. (2013)]

The target molecule can be any molecule associated with a human ailment or disorder that is present on the skin or in the esophagus, nasal tissue, mouth, vaginal tissue, lungs, stomach, small intestines, large intestines, digestive tract, or gastrointestinal tract of said subject. The target molecule can be any molecule associated with human disease or ailment that is present within the gastrointestinal tract of a subject. Exemplary, non-limiting, target molecules can include: hormones, metabolites produced by microorganisms, nutrients and/or dietary components, microorganism quorum sensing molecules, inflammatory molecules,

The target molecule can be any molecule associated with sleep regulation in a subject. The target molecule can be any molecule associated with mood regulation in a subject. The target molecule can be any molecule associated with appetite control in a subject. The target molecule can be any molecule associated with pain relief and/or pleasure in a subject.

The target molecule can be any molecule associated with social bonding in a subject.

The target molecule can comprise cortisol, insulin, peptide YY, melatonin, dopamine, serotonin, ghrelin, leptin, oxytocin, endorphins, glucose, lactose, gluten, phenols, histamine, reactive oxygen species, vitamins (e.g., Vitamin D, Vitamin B12), minerals (e.g., iron, magnesium), amino acids (e.g., tryptophan), omega-3 fatty acids, short-chain fatty acids (e.g., butyrate, propionate), lactate, Ketone Bodies, natural phytochemicals (e.g., flavonoids, antioxidants), cytokines, and/or any microorganism-specific molecule.

A microorganism-specific molecule can be a molecule that is known to be associated with a particular microorganism, which can effectively be used to identify said microorganism due to its presence. That is, no other known microorganism would be understood to be associated with said molecule. The molecule can be for example, a surface protein and/or marker associated with a pathogenic microorganism, such as E. coli, Clostridioides difficile, Clostridium perfringens, or any known pathogenic microorganism. Pathogenic microorganisms would be readily knonw by a person having skill in the art.

In a particular embodiment, the target molecule comprises cortisol. Cortisol levels can be elevated in any tissue of a subject. Cortisol can be bound in any tissue of a subject. In some instances, the target molecule comprises cortisol, wherein said cortisol is produced by the subject, and is elevated in at least one of the following tissue of said subject: on the skin or in the esophagus, nasal tissue, mouth, vaginal tissue, lungs, stomach, small intestines, large intestines, digestive tract, or gastrointestinal tract of said subject.

The biosensing compositions disclosed herein can comprise a plurality of engineered yeast comprising a molecule responsive element comprising a binding domain that interacts with a target molecule, or a polynucleotide comprising a nucleotide sequence encoding the molecule responsive element, and a nucleic acid molecule comprising a polynucleotide encoding a response factor, wherein said target molecule comprises cortisol.

In a particular embodiment, the target molecule comprises histamine. Histamine levels can be elevated in any tissue of a subject. Histamine can be bound in any tissue of a subject. In some instances, the target molecule comprises histamine, wherein said histamine is produced by the subject, and is elevated in at least one of the following tissue of said subject: on the skin or in the esophagus, nasal tissue, mouth, vaginal tissue, lungs, stomach, small intestines, large intestines, digestive tract, or gastrointestinal tract of said subject.

The biosensing compositions disclosed herein can comprise a plurality of engineered yeast comprising a molecule responsive element comprising a binding domain that interacts with a target molecule, or a polynucleotide comprising a nucleotide sequence encoding the molecule responsive element, and a nucleic acid molecule comprising a polynucleotide encoding a response factor, wherein said target molecule comprises histamine.

Some aspects of this disclosure provide molecule responsive elements that can be introduced into a yeast cell for detecting and/or binding of any target molecule disclosed herein. The molecule responsive elements can comprise a polypeptide comprising a binding domain that interacts with a target molecule. Such molecule responsive elements are useful for binding target molecules in vitro, ex vivo, or in vivo.

The molecule responsive elements as described herein can comprise, but are not limited to, polypeptides comprising an amino acid sequence, such as fusion proteins, receptors, or transmembrane proteins. In certain embodiments, the receptors and/or proteins can be naturally-occurring, recombinant or synthetic. “Recombinant” or “synthetic” refers to a responsive element that is at least partially man-made, or not naturally-occurring. In such instances, a component of the responsive element is recombinant, or heterologous to the host cell in which it is being produced. In some instances, the molecule responsive element comprises a human protein, such as a human receptor. The molecule responsive elements described herein will be capable of being expressed and produced in engineered yeast of the present disclosure. For instance, an engineered yeast cell can comprise a polynucleotide comprising a nucleotide sequence encoding said molecule responsive element, wherein said polynucleotide is codon-optimized for expression in yeast cells. The polynucleotide comprising any molecule responsive element can be codon-optimized for expression in yeast cells.

Disclosed herein are molecule responsive elements capable of interacting with a target molecule. A molecule responsive element comprises a binding domain that enables the element to interact with a target molecule. In some instances, the molecule responsive element binds to a target molecule. A molecule responsive element may interact with a target molecule through any chemical or physical method of interaction between two molecules. The molecule responsive elements disclosed herein can comprise a binding domain that interacts with a target molecule disclosed herein. There may be particular target molecules of interest for interacting with a molecule responsive element, for which a specific binding domain is engineered into the molecule responsive element. In such instances, the molecule responsive element can be a recombinant and/or synthetic molecule that is engineered with a binding domain to interact with a particular target molecule. The molecule responsive element can comprise a polypeptide or a nucleic acid molecule. In some instances, the molecule responsive element comprises both a polypeptide and a nucleic acid molecule.

In a specific embodiment, the molecule responsive element comprises a polypeptide. The molecule responsive element can be a polypeptide. Also disclosed are polynucleotides encoding the molecule responsive elements disclosed herein. For instance, when the molecule responsive element comprises a polypeptide, or is a polypeptide, the polynucleotides comprise a nucleic acid sequence encoding the polypeptide. The molecule responsive elements can also comprise a nucleic acid molecule. In some instances, the molecule responsive element is a nucleic acid molecule. Any nucleic acid molecule that comprises a binding domain capable of interacting with a target molecule disclosed herein is envisaged within the scope of the presently disclosed molecule responsive elements. For instance, the nucleic acid molecule can comprise DNA, RNA, or both. The DNA and/or RNA can be naturally occurring, recombinant DNA and/or RNA, and/or synthetic DNA and/or RNA. In some instances, the nucleic acid molecule comprised within the molecule responsive element comprises both a naturally occurring and recombinant nucleic acid.

The molecule responsive elements disclosed herein can comprise a polypeptide sequence. The polypeptide sequence can comprise an amino acid sequence encoding a binding domain that interacts with a target molecule. Binding domains that interact with, recognize, and/or bind to any target molecule disclosed herein are envisaged within the scope of the binding domains present within the molecule responsive elements of the present application. The binding domain can be specific for a target molecule or class of target molecule or can be capable of binding and responding to several target molecules or classes of target molecules. In some embodiments, the receptor can be a toll-like receptor (TLR), Pregnane X Receptor (PXR), Bitter Taste Receptor (TAS2Rs), receptors that detect N-formyl peptides, rs (PPARs), or any other applicable receptor known in the art.

A binding domain disclosed herein can include functional fragments and/or variants of the binding domain. A polypeptide disclosed herein can include functional fragments and/or variants of the polypeptide. A “functional fragment” or “functional variant” maintains the ability to bind to or interact with a target molecule. A functional fragment and/or variant of a binding domain will comprise the residues necessary from the parent binding domain to interact with the target molecule. In some instances, the molecule responsive element comprises a binding domain that interacts with cortisol (i.e., a cortisol binding domain), or a functional fragment or variant of a binding domain that interacts with cortisol. In such instances, the molecule responsive element can comprise a GC receptor. In some instances, the molecule responsive element can comprise a functional fragment and/or variant of a GC receptor. The molecule responsive element can comprise a functional fragment and/or variant of a human GC receptor. In an additional embodiment, the molecule responsive element comprises a binding domain that interacts with histamine (i.e, a histamine binding domain), or a functional fragment or variant of a binding domain that interacts with histamine. In such instances, the molecule responsive element can comprise a histamine receptor. In some instances, the molecule responsive element can comprise a functional fragment and/or variant of a histamine receptor. The molecule responsive element can comprise a functional fragment and/or variant of a human histamine receptor. When the molecule responsive element comprises a polypeptide, the polypeptide can comprise a fusion protein, a transmembrane protein, a receptor, or a functional fragment thereof. The functional fragment of a polypeptide can have at least 80%, at least 85%, at least 90%, at least 95%, or at least 99% sequence identity to the parent polypeptide. A functional fragment and/or variant of a polypeptide will retain the activity of the parent polypeptide. For instance, if the parent polypeptide interacts with or binds to a target molecule, then the functional fragment will be capable of interacting with and/or binding to the same target molecule. That is, a functional fragment and/or variant of a GC receptor retains the capacity to interact with cortisol. Alternatively, a functional fragment and/or variant of a histamine receptor retains the capacity to interact with histamine. One key aspect of the molecule responsive element is the ability to modify the expression, activity, or amount of a response factor. In some instances, interaction of a molecule responsive element with a target molecule can initiate activation of downstream gene expression of a response factor. As such, a functional fragment and/or variant of the polypeptide retains the ability to modify expression, activity, or amount of a response factor, for instance, initiate expression of a gene encoding, or production and/or secretion of, a response factor upon interaction with a target molecule. As described herein, production of a response factor can be increased following an increase in expression of a gene encoding said response factor. A functional fragment and/or variant of a GC receptor retains the capacity to modify the expression, activity, or amount of a response factor upon interaction with cortisol. Additionally, a functional fragment and/or variant of a histamine receptor retains the capacity to modify the expression, activity, or amount of a response factor upon interaction with histamine. The molecule responsive elements disclosed herein can increase expression of a gene encoding a response factor, directly or indirectly. Direct activation of gene expression would be understood to involve direct interaction of the molecule responsive factor, or a region thereof, to the nucleic acid molecule encoding the response element. Indirect activation of gene expression would be understood to involve a second molecule, other than the molecule responsive element, to increase gene expression of the response factor encoding gene. In some instances, the molecule responsive element comprises a fusion protein, or a functional fragment thereof. In some instances, the molecule responsive element comprises a transmembrane protein, or a functional fragment thereof. In some instances, the molecule responsive element comprises a receptor, or a functional fragment thereof.

The molecule responsive element can comprise a receptor. Any receptor, or variant or fragment thereof, capable of interacting with a target molecule and increasing expression of a gene encoding, or production and/or secretion of, a response factor is envisaged within the scope of the present application. As the disclosed molecule responsive elements are particularly effective at binding, detecting and/or reducing the levels of a human target molecule, the molecule responsive element can comprise a molecule derived from a human. For instance, the molecule responsive element can comprise a human polypeptide, or a fragment or variant thereof. Alternatively, the molecule responsive element can comprise a human nucleic acid molecule, or a fragment or variant thereof. When the molecule responsive element comprises a human polypeptide, or a fragment or variant thereof, any human polypeptide can be used. For instance, a human receptor, transmembrane protein, and/or transcription factor can be used. In some instances, the molecule responsive element comprises a fusion protein comprises a human protein or fragment or variant thereof.

In a particular embodiment of the presently disclosed biosensing composition, the molecule responsive element comprises a human glucocorticosteroid (GC) receptor. The molecule responsive element comprising a human GC receptor can comprise at least 80%, at least 85%, at least 90%, at least 95%, at least 99%, or 100% sequence identity to a human GC receptor.

The molecule responsive element disclosed herein can comprise a human receptor, or a functional fragment or variant thereof. The receptor can comprise a glucocorticosteroid (GC) receptor. The GC receptor (GR or GCR), also known as NR3C1 (nuclear receptor subfamily 3, group C, member 1), is the receptor to which cortisol and other glucocorticoids bind. The GC receptor is expressed in almost every cell in the body and regulates genes controlling the development, metabolism, and immune response. Glucocorticoids are steroid hormones secreted by the adrenal glands. They are important for the maintenance of basal and stress-related homeostasis by acting as end products of the stress-responsive hypothalamic-pituitary-adrenal (HPA) axis. Glucocorticoids regulate a variety of biologic processes and exert profound influences on many physiologic functions. In pharmacologic doses, glucocorticoids are used as potent immunosuppressive agents in the therapeutic management of many inflammatory, autoimmune and lympho-proliferative diseases (4). At the cellular level, the actions of glucocorticoids are mediated by an intracellular receptor protein, the glucocorticoid receptor (GR) (its gene name is “nuclear receptor subfamily 3, group C, member 1: NR3C1”), which belongs to the steroid/sterol/thyroid/retinoid/orphan receptor superfamily of nuclear transactivating factors with over 200 members in general and over 40 in mammals currently cloned and characterized across species, as described in Mangelsdorf D J, Thummel C, Beato M, Herrlich P, Schutz G, Umesono K, Blumberg B, Kastner P, Mark M, Chambon P, Evans R M. The nuclear receptor superfamily: the second decade. Cell. 1995 Dec. 15; 83(6):835-9, the contents of which are incorporated herein in their entirety. Human GR consists of 777 amino acid residues (UniProt Accession Number F1D8N4).

A human GC receptor (NCBI Accession No. NP_000167.1) can comprise the following amino acid sequence:

(SEQ ID NO: 1)
MDSKESLTPGREENPSSVLAQERGDVMDFYKTLRGGATVKVSASS
PSLAVASQSDSKQRRLLVDFPKGSVSNAQQPDLSKAVSLSMGLYM
GETETKVMGNDLGFPQQGQISLSSGETDLKLLEESIANLNRSTSV
PENPKSSASTAVSAAPTEKEFPKTHSDVSSEQQHLKGQTGTNGGN
VKLYTTDQSTFDILQDLEFSSGSPGKETNESPWRSDLLIDENCLL
SPLAGEDDSFLLEGNSNEDCKPLILPDTKPKIKDNGDLVLSSPSN
VTLPQVKTEKEDFIELCTPGVIKQEKLGTVYCQASFPGANIIGNK
MSAISVHGVSTSGGQMYHYDMNTASLSQQQDQKPIFNVIPPIPVG
SENWNRCQGSGDDNLTSLGTLNFPGRTVFSNGYSSPSMRPDVSSP
PSSSSTATTGPPPKLCLVCSDEASGCHYGVLTCGSCKVFFKRAVE
GQHNYLCAGRNDCIIDKIRRKNCPACRYRKCLQAGMNLEARKTKK
KIKGIQQATTGVSQETSENPGNKTIVPATLPQLTPTLVSLLEVIE
PEVLYAGYDSSVPDSTWRIMTTLNMLGGRQVIAAVKWAKAIPGFR
NLHLDDQMTLLQYSWMFLMAFALGWRSYRQSSANLLCFAPDLIIN
EQRMTLPCMYDQCKHMLYVSSELHRLQVSYEEYLCMKTLLLLSSV
PKDGLKSQELFDEIRMTYIKELGKAIVKREGNSSQNWQRFYQLTK
LLDSMHEVVENLLNYCFQTFLDKTMSIEFPEMLAEIITNQIPKYS
NGNIKKLLFHQK.

Disclosed herein are biosensing compositions for detecting a target molecule in a subject, comprising a plurality of engineered yeast cells comprising a polypeptide encoding a human glucocorticosteroid (GC) receptor, or a functional fragment or variant thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to a human glucocorticosteroid (GC) receptor.

Disclosed herein are biosensing compositions for detecting a target molecule in a subject, comprising a plurality of engineered yeast cells comprising a polypeptide encoding a human glucocorticosteroid (GC) receptor, or a functional fragment or variant thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 1 (i.e., amino acid sequence of human GC receptor (NCBI Accession No. NP_000167.1)). In some instances, the plurality of engineered yeast cells comprises a polypeptide encoding a human glucocorticosteroid (GC) receptor comprising an amino acid sequence set forth as SEQ ID NO: 1.

Disclosed herein are biosensing compositions for detecting a target molecule in a subject, comprising a plurality of engineered yeast cells comprising a polynucleotide encoding a human glucocorticosteroid (GC) receptor, or a functional fragment or variant thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 1. The polynucleotide can be codon-optimized for expression in yeast. Disclosed herein are biosensing compositions for detecting a target molecule in a subject, comprising a plurality of engineered yeast cells comprising a polynucleotide encoding a human glucocorticosteroid (GC) receptor, wherein said human glucocorticosteroid (GC) receptor is encoded by a nucleotide sequence set forth as SEQ ID NO: 2. In some instances, the plurality of engineered yeast cells comprises a polynucleotide encoding a human glucocorticosteroid (GC) receptor, wherein said human glucocorticosteroid (GC) receptor is encoded by a nucleotide sequence set forth as SEQ ID NO: 2.

In some instances, the engineered yeast of the present disclosure comprises a molecule responsive element, or a polynucleotide sequence comprising a nucleotide sequence encoding a molecule responsive element, wherein said molecule responsive element comprises a polypeptide sequence comprising an amino acid sequence comprising a glucocorticosteroid (GC) receptor. In some instances, the molecule responsive element can comprise a polypeptide sequence comprising a human glucocorticosteroid (GC) receptor. In some instances, the molecule responsive element can comprise a polypeptide sequence comprising a human glucocorticosteroid (GC) receptor, or a functional fragment or variant thereof having at least 85%, at least 90%, at least 95%, or at least 99% sequence identity to a human glucocorticosteroid (GC) receptor. In some instances, the engineered yeast comprises a recombinant glucocorticosteroid (GC) receptor. In some instances, the recombinant glucocorticosteroid (GC) receptor has at least 85%, at least 90%, at least 95%, or at least 99% sequence identity to a human glucocorticosteroid (GC) receptor, wherein the recombinant glucocorticosteroid (GC) receptor retains the ability to interact with cortisol and activate downstream gene expression (e.g., interact with a GC receptor-activated promoter and/or activate expression of a gene operably linked to a GC-receptor-activated promoter).

Human GC receptor functions as a hormone-dependent transcription factor that regulates the expression of glucocorticoid-responsive genes, which probably represent 3-10% of the human genome and can be influenced by the ligand-activated GR directly or indirectly.

The molecule responsive element can comprise a polypeptide sequence comprising an amino acid sequence set forth as SEQ ID NO: 1, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 1. In some instances, the molecule responsive element is expressed in a plurality of engineered yeast cells. In such instances, the molecule responsive element comprising a polypeptide sequence comprising an amino acid sequence set forth as SEQ ID NO: 1, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 1 can be codon-optimized for expression in yeast. In some embodiments, the nucleotide sequence is codon-optimized for expression in S. cerevisiae. In such instances, the molecule responsive element can be encoded by a nucleotide sequence set forth as SEQ ID NO: 2. The molecule responsive element can be encoded by a polynucleotide comprising a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 2.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule to said binding domain results in production and/or secretion of said response factor in said engineered yeast cells, wherein said molecule responsive element comprises a human glucocorticosteroid (GC) receptor comprising an amino acid sequence set forth as SEQ ID NO: 1, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 1.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor comprising an amino acid sequence set forth as SEQ ID NO: 1, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 1, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule to said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 2.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 2, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter.

In any of the compositions disclosed herein, wherein the plurality of engineered yeast comprises a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, the activation domain can comprise a polynucleotide sequence set forth as: a. SEQ ID NO: 5, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polynucleotide sequence set forth as SEQ ID NO: 5; b. SEQ ID NO: 6, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polynucleotide sequence set forth as SEQ ID NO: 6; and/or c. SEQ ID NO: 11, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polynucleotide sequence set forth as SEQ ID NO: 11. A variant when used to refer to the activation domain operably linked to the polynucleotide sequence encoding said response factor is understood to be a sequence that retains the ability to interact with any molecule(s) necessary (e.g., GC receptor and/or transcription factors) to initiate and drive gene expression (i.e., transcription) of the response factor to which it is operably linked.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor comprising an amino acid sequence set forth as SEQ ID NO: 1, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein the activation domain comprises a polynucleotide sequence set forth as: a. SEQ ID NO: 5.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor comprising an amino acid sequence set forth as SEQ ID NO: 1, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein the activation domain comprises a polynucleotide sequence set forth as: a. SEQ ID NO: 5, wherein said yeast cells comprise Saccharomyces.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor comprising an amino acid sequence set forth as SEQ ID NO: 1, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein the activation domain comprises a polynucleotide sequence set forth as: a. SEQ ID NO: 6.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor comprising an amino acid sequence set forth as SEQ ID NO: 1, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein the activation domain comprises a polynucleotide sequence set forth as: a. SEQ ID NO: 6, wherein said yeast cells comprise Saccharomyces.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor comprising an amino acid sequence set forth as SEQ ID NO: 1, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein the activation domain comprises a polynucleotide sequence set forth as SEQ ID NO: 11.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor comprising an amino acid sequence set forth as SEQ ID NO: 1, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein the activation domain comprises a polynucleotide sequence set forth as: a. SEQ ID NO: 11, wherein said yeast cells comprise Saccharomyces.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein said activation domain comprises a polynucleotide sequence set forth as SEQ ID NO: 5.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein said activation domain comprises a polynucleotide sequence set forth as SEQ ID NO: 5, wherein said yeast cells comprise Saccharomyces.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein said activation domain comprises a polynucleotide sequence set forth as SEQ ID NO: 6.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein said activation domain comprises a polynucleotide sequence set forth as SEQ ID NO: 6, wherein said yeast cells comprise Saccharomyces.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein said activation domain comprises a polynucleotide sequence set forth as SEQ ID NO: 11.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a human glucocorticosteroid (GC) receptor encoded by a nucleotide sequence set forth as SEQ ID NO: 2, wherein said nucleic acid molecule comprising a polynucleotide sequence encoding a response factor comprises an activation domain operably linked to the polynucleotide sequence encoding said response factor, wherein said activation domain comprises a promoter comprising a human glucocorticosteroid response element (HRE)-activated promoter, wherein said activation domain comprises a polynucleotide sequence set forth as SEQ ID NO: 11, wherein said yeast cells comprise Saccharomyces.

The molecule responsive element can comprise a fusion protein, such as a recombinant fusion protein. A recombinant fusion protein can comprise a sequence that is naturally, occurring in addition to a recombinant sequence. The naturally, occurring sequence can account for any portion of the recombinant fusion protein. In some instances, the fusion protein is no more than 95%, 90%, 85%, 80%, 75%, 70%, 65%, 60%, 50%, 45%, 40%, 35%, 30%, 25%, 20%, 15%, 10%, 5%, or 1% recombinant sequence. The fusion protein can comprise 100% recombinant sequence. In some instances, the molecule responsive element can comprise a transmembrane protein. In such instances, the molecule responsive element can comprise a fusion protein comprising a transmembrane protein, or a functional fragment or variant thereof. The transmembrane protein can be a recombinant transmembrane protein. Alternatively, the transmembrane protein can be a naturally, occurring transmembrane protein. The transmembrane protein can be any naturally, occurring transmembrane protein, or can comprise a portion of any naturally, occurring transmembrane protein. The naturally, occurring transmembrane protein, or portion thereof, can be from any species, or combination of species.

In a particular embodiment of the presently disclosed biosensing composition, the molecule responsive element comprises a human G-protein coupled receptor (GPCR). The molecule responsive element comprising a human G-protein coupled receptor (GPCR) can comprise at least 80%, at least 85%, at least 90%, at least 95%, at least 99%, or 100% sequence identity to a human GPCR. In a particular embodiment of the presently disclosed biosensing composition, the molecule responsive element comprises a human G-protein coupled receptor (GPCR) that binds histamine. The molecule responsive element comprising a human G-protein coupled receptor (GPCR) that binds histamine can comprise at least 80%, at least 85%, at least 90%, at least 95%, at least 99%, or 100% sequence identity to a human GC receptor that binds histamine. The molecule responsive element comprising a human G-protein coupled receptor (GPCR) that binds histamine can comprise at least 90%, at least 91%, at least 92%, at least 93%, at least 94%, at least 95%, at least 96%, at least 97%, at least 98%, or at least 99% sequence identity to a human GC receptor that binds histamine. In some instances, the molecule responsive element comprises the binding domain of a GPCR receptor that binds histamine, or a functional fragment or variant having at least 80%, at least 85%, at least 90%, at least 95%, at least 99%, or 100% sequence identity to a human GC receptor histamine binding domain.

The fusion protein and/or transmembrane protein can comprise a G-protein coupled receptor (GPCR). The G-protein coupled receptor (GPCR) can be a recombinant protein. For instance, the fusion protein and/or transmembrane protein can comprise a recombinant G-protein coupled receptor (GPCR) fusion protein.

G protein coupled receptors (GPCRs) represent the largest family of membrane proteins in the human genome. A recent and detailed analysis of the human genome reveals over 800 unique GPCRs, of which approximately 460 are predicted to be olfactory receptors, as described in Fredriksson R, Lagerström M C, Lundin L G, Schiöth H B. Common structural features and functions of GPCRs would be understood by one having skill in the art. Based on sequence similarity within the 7 TM segments, these receptors can be clustered into 5 families: the rhodopsin family (701 members), the adhesion family (24 members), the frizzled/taste family (24 members), the glutamate family (15 members), and the secretin family (15 members) (Fredriksson R, Lagerström M C, Lundin L G, Schiöth H B. The G-protein-coupled receptors in the human genome form five main families. Phylogenetic analysis, paralogon groups, and fingerprints. Mol Pharmacol. 2003 June; 63(6):1256-72). The physiologic function of a large fraction of these 800 GPCRs is unknown; these receptors are referred to as orphan GPCRs. GPCRs share a common structural signature of seven hydrophobic transmembrane (TM) segments, with an extracellular amino terminus and an intracellular carboxyl terminus. GPCRs share the greatest homology within the TM segments. The most variable structures among the family of GPCRs are the carboxyl terminus, the intracellular loop spanning TM5 and TM6, and the amino terminus. The greatest diversity is observed in the amino terminus. This sequence is relatively short (10-50 amino acids) for monoamine and peptide receptors, and much larger (350-600 amino acids) for glycoprotein hormone receptors, and the glutamate family receptors. The largest amino terminal domains are observed in the adhesion family receptors. In spite of the remarkable diversity of ligands and ligand binding domains in the family of GPCRs, there is also considerable evidence for a common mechanism of activation. When comparing sequences, GPCRs are most similar at the cytoplasmic ends of the transmembrane segments adjacent to the second and third cytoplasmic domains, the regions known to interact with cytoplasmic G proteins, as described in Mirzadegan T, Benko G, Filipek S, Palczewski K. Sequence analyses of G-protein-coupled receptors: similarities to rhodopsin. Biochemistry. 2003 Mar. 18; 42(10):2759-67, the contents of which are incorporated herein by reference in their entirety. Members of the large family of GPCRs transduce signals by activating one or more members of the relatively small family of highly homologous heterotrimeric G proteins. For example, the follicle stimulating hormone (FSH) receptor is activated by a large glycoprotein hormone that binds to the amino terminus while the β2AR is activated by adrenaline (approximately the size of a single amino acid) that binds to the TM segments; yet both of these receptors activate the same G protein (Gs), indicating that the structural changes in the cytoplasmic domains of these two receptors must be very similar. Moreover, many GPCRs exhibit promiscuous coupling to more than one G protein. For example, rhodopsin preferentially couples to transducin while the β2AR preferentially couples to Gs; however, both are capable of activating Gi.

In some instances, the molecule responsive element comprises a polypeptide sequence that comprises a histamine receptor. Histamine is an important tissue hormone which is primarily detected by human cells via the four histamine receptors H1R, H2R, H3R and H4R, as described in Parsons M E, Ganellin C R. Histamine and its receptors. Br J Pharmacol. 2006 January; 147 Suppl 1(Suppl 1):S127-35, and Seifert R, Strasser A, Schneider E H, Neumann D, Dove S, Buschauer A. Molecular and cellular analysis of human histamine receptor subtypes. Trends Pharmacol Sci. 2013 January; 34(1):33-58, the contents of which are incorporated herein by reference in their entirety. All of them belong to the class A of G protein-coupled receptors (GPCRs), described in Rosenbaum D M, Rasmussen S G, Kobilka B K. The structure and function of G-protein-coupled receptors. Nature. 2009 May 21; 459(7245):356-63, the contents of which are incorporated herein by reference in their entirety. Binding of histamine favors the transition from inactive towards active subsets of receptor conformations which allows for the coupling to intracellular binding partners (IBPs). Each histamine receptor couples to a specific subset of IBPs including several types of G proteins and arrestins, which participate in a plethora of different pathways and thus lead to various changes within the behavior of the cell and the surrounding tissue.

A human histamine receptor (NCBI Accession No. NP_000852) can comprise the following amino acid sequence:

(SEQ ID NO: 3)
MSLPNSSCLLEDKMCEGNKTTMASPQLMPLVVVLSTICLVTVGLN
LLVLYAVRSERKLHTVGNLYIVSLSVADLIVGAVVMPMNILYLLM
SKWSLGRPLCLFWLSMDYVASTASIFSVFILCIDRYRSVQQPLRY
LKYRTKTRASATILGAWFLSFLWVIPILGWNHFMQQTSVRREDKC
ETDFYDVTWFKVMTAIINFYLPTLLMLWFYAKIYKAVRQHCQHRE
LINRSLPSFSEIKLRPENPKGDAKKPGKESPWEVLKRKPKDAGGG
SVLKSPSQTPKEMKSPVVFSQEDDREVDKLYCFPLDIVHMQAAAE
GSSRDYVAVNRSHGQLKTDEQGLNTHGASEISEDQMLGDSQSFSR
TDSDTTTETAPGKGKLRSGSNTGLDYIKFTWKRLRSHSRQYVSGL
HMNRERKAAKQLGFIMAAFILCWIPYFIFFMVIAFCKNCCNEHLH
MFTIWLGYINSTLNPLIYPLCNENFKKTFKRILHIRS.

Disclosed herein are biosensing compositions for detecting a target molecule in a subject, comprising a plurality of engineered yeast cells comprising a polypeptide encoding a human G-protein coupled receptor (GPCR) protein, or a functional fragment or variant thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to a human G-protein coupled receptor (GPCR) protein.

Disclosed herein are biosensing compositions for detecting a target molecule in a subject, comprising a plurality of engineered yeast cells comprising a polypeptide encoding a human G-protein coupled receptor (GPCR) protein, or a functional fragment or variant thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 3 (i.e., amino acid sequence of histamine receptor (NCBI Accession No. NP_000852)). In some instances, the plurality of engineered yeast cells comprises a polypeptide encoding a human G-protein coupled receptor (GPCR) protein comprising an amino acid sequence set forth as SEQ ID NO: 3.

Disclosed herein are biosensing compositions for detecting a target molecule in a subject, comprising a plurality of engineered yeast cells comprising a polynucleotide encoding a human G-protein coupled receptor (GPCR) protein, or a functional fragment or variant thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 3. The polynucleotide can be codon-optimized for expression in yeast. Disclosed herein are biosensing compositions for detecting a target molecule in a subject, comprising a plurality of engineered yeast cells comprising a polynucleotide encoding a human G-protein coupled receptor (GPCR) protein, wherein said human G-protein coupled receptor (GPCR) protein is encoded by a nucleotide sequence set forth as SEQ ID NO: 4. In some instances, the plurality of engineered yeast cells comprises a polynucleotide encoding a human G-protein coupled receptor (GPCR) protein, wherein said human G-protein coupled receptor (GPCR) protein is encoded by a nucleotide sequence set forth as SEQ ID NO: 4.

In some instances, the engineered yeast of the present disclosure comprises a molecule responsive element, or a polynucleotide sequence comprising a nucleotide sequence encoding a molecule responsive element, wherein said molecule responsive element comprises a polypeptide sequence comprising an amino acid sequence encoding a histamine receptor. In some instances, the molecule responsive element can comprise a polypeptide sequence comprising a histamine receptor that is heterologous to yeast. In some instances, the molecule responsive element can comprise a polypeptide sequence comprising a human histamine receptor. In some instances, the molecule responsive element can comprise a polypeptide sequence comprising a human histamine receptor, or a functional fragment or variant thereof having at least 85%, at least 90%, at least 95%, or at least 99% sequence identity to a human histamine receptor. In some instances, the engineered yeast comprises a recombinant histamine receptor. In some instances, the recombinant histamine receptor has at least 85%, at least 90%, at least 95%, or at least 99% sequence identity to a human histamine receptor, wherein the recombinant histamine receptor retains the ability to interact with histamine and activate downstream gene expression (e.g., activate downstream signaling pathway).

The molecule responsive element can comprise a fusion protein comprising any GPCR, or a functional fragment or variant thereof. For instance, the fusion protein can comprise a naturally, occurring GPCR, or can comprise a portion of any naturally, occurring GPCR. In some instances, the molecule responsive element comprises a GPCR protein, or a functional fragment or variant thereof, that comprises a histamine receptor. In some instances, the molecule responsive element can comprise a histamine binding domain of a human GPCR. The molecule responsive element can comprise a polypeptide sequence comprising an amino acid sequence encoding a histamine binding domain of a human GPCR set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3.

In some instances, the molecule responsive element is expressed in a plurality of engineered yeast cells. In such instances, the molecule responsive element comprising a polypeptide sequence comprising an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3 can be codon-optimized for expression in yeast. In some embodiments, the nucleotide sequence is codon-optimized for expression in S. cerevisiae. In such instances, the molecule responsive element can be encoded by a nucleotide sequence set forth as SEQ ID NO: 4. The molecule responsive element can be encoded by a polynucleotide comprising a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 4.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain and is encoded by a nucleotide sequence set forth as SEQ ID NO: 4, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 4.

Disclosed herein are biosensing compositions for detecting a target molecule in a subject, comprising a plurality of engineered yeast cells, comprising: a. a polynucleotide comprising a nucleotide sequence encoding a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain and is encoded by a nucleotide sequence set forth as SEQ ID NO: 4, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 4, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter.

In some instances, the molecule responsive element can comprise a polypeptide sequence that is heterologous to yeast. In such instances, the molecule responsive element can comprise a histamine receptor that is heterologous to yeast. For instance, the molecule responsive element can comprise a polypeptide sequence encoding a human histamine receptor. When a human histamine receptor is expressed in yeast, a person having ordinary skill in the art would readily understand that it may be necessary for the yeast to express a recombinant signaling molecule capable of interacting with the heterologous human histamine receptor. In some instances, wherein the engineered yeast comprises a molecule responsive element comprising a human histamine receptor, the engineered yeast further comprises a recombinant G protein molecule capable of interacting with the heterologous receptor to mediate signal transduction from the human histamine receptor within the engineered yeast cell. In some instances, a recombinant G protein can be expressed in a yeast cell through the introduction of at least one genetic alteration or mutation within a native G protein gene. Any genetic alteration or mutation within a G protein encoding gene that enables the encoded recombinant G protein to interact with a heterologous histamine receptor and mediate signaling transduction is envisaged within the scope of the present application.

In some instances, the engineered yeast comprises a genomic modification in a native gene encoding a yeast G protein. For example, a native gene encoding a yeast G protein can comprise one or more than one nucleotide substitution, insertion, and/or deletion, or the native gene can be fully deleted from the chromosome. In such embodiments, a polynucleotide encoding a recombinant G protein can be introduced into the engineered yeast, whereby said recombinant G protein is capable of interacting with the heterologous histamine receptor within the yeast to mediate signal transduction. In such instances, the gene encoding the native G protein can be fully removed (i.e., deleted). Methods of genetically modifying yeast are known in the art. Any method for introducing a genetic alteration into yeast can be used.

Disclosed herein are modified GPA1 proteins capable of interacting with a human histamine receptor to mediate signal transduction when the histamine receptor is expressed in yeast. In some instances, the modified GPA1 proteins can interact with a human histamine receptor comprising an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, within an engineered yeast cell.

Disclosed herein are biosensing compositions for detecting a target molecule in a subject, comprising a plurality of engineered yeast cells, comprising: a. a molecule responsive element comprising binding domain that interacts with said target molecule, or a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, or a polynucleotide comprising a nucleotide sequence encoding a modified GPA1 protein, wherein said modified GPA1 protein comprises an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8.

Disclosed herein are biosensing compositions for detecting a target molecule in a subject, comprising a plurality of engineered yeast cells, comprising: a. a molecule responsive element comprising binding domain that interacts with said target molecule, or a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a polynucleotide comprising a nucleotide sequence encoding a modified GPA1 protein set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

In any of the embodiments described herein, the polynucleotide comprising a nucleotide sequence encoding a modified GPA1 protein can be present on the chromosome of the engineered yeast and/or present extra-chromosomally (e.g., on a plasmid). In some instances, the engineered yeast comprising the polynucleotide encoding a modified GPA1 protein comprises one or more than one genomic modification within a native GPA1 gene. For example, the engineered yeast may comprise a GPA1 gene, or variant thereof, comprising one or more than one nucleotide alteration (e.g., substitution), insertion and/or deletion compared to the native GPA1 gene.

Disclosed herein are biosensing compositions for detecting a target molecule in a subject, comprising a plurality of engineered yeast cells comprising at least one genomic modification within a native GPA1 gene. In some instances, the engineered yeast comprises more than one genomic modification with a native GPA1 gene. In some instances, the engineered yeast comprises a full deletion of a native GPA1 gene. In such instances, wherein the engineered yeast comprises one or more than one genomic modification of a native GPA1 gene, the GPA1 encoding gene (i.e., modified GPA1 gene) encodes a modified GPA1 protein comprising one or more than one amino acid modification (i.e., amino acid substitution, insertion, and/or deletion) compared to a native GPA1 protein. The engineered yeast can comprise a polynucleotide comprising a nucleotide sequence encoding a modified GPA1 gene that encodes for a modified (e.g., recombinant) GPA1 protein.

The native GPA1 gene (encoding guanine nucleotide-binding protein GP1-alpha) in S. cerevisiae comprises the following nucleotide sequence:

(SEQ ID NO: 9; NCBI Accession No. M15867)
ATGGGGTGTACAGTGAGTACGCAAACAATAGGAGACGAAAGTGAT
CCTTTTCTACAGAACAAAAGAGCCAATGATGTCATCGAGCAATCG
TTGCAGCTGGAGAAACAACGTGACAAGAATGAAATAAAACTGTTA
CTATTAGGTGCCGGTGAGTCAGGTAAATCAACGGTTTTAAAACAA
TTAAAATTATTACATCAAGGCGGTTTCTCCCATCAAGAAAGGTTA
CAGTATGCTCAAGTGATATGGGCAGATGCCATACAATCAATGAAA
ATTTTGATTATTCAGGCCAGAAAACTAGGTATTCAACTTGACTGT
GATGATCCGATCAACAATAAAGATTTGTTTGCATGCAAGAGAATA
CTGCTAAAGGCTAAAGCTTTAGATTATATCAACGCCAGTGTTGCC
GGTGGTTCTGATTTTCTAAATGATTATGTACTGAAGTACTCAGAA
AGGTATGAAACTAGGAGGCGTGTTCAGAGTACCGGACGAGCAAAA
GCTGCTTTCGATGAAGACGGAAATATTTCTAATGTCAAAAGTGAC
ACTGACAGAGATGCTGAAACGGTGACGCAAAATGAGGATGCTGAT
AGAAACAACAGTAGTAGAATTAACCTACAGGATATTTGCAAGGAC
TTGAACCAAGAAGGCGATGACCAGATGTTTGTTAGAAAAACATCA
AGGGAAATTCAAGGACAAAATAGACGAAATCTTATTCACGAAGAC
ATTGCTAAGGCAATAAAGCAACTTTGGAATAACGACAAAGGTATA
AAGCAGTGTTTTGCACGTTCTAATGAGTTTCAATTGGAGGGCTCA
GCTGCATACTACTTTGATAACATTGAGAAATTTGCTAGTCCGAAT
TATGTCTGTACGGATGAAGACATTTTGAAGGGCCGTATAAAGACT
ACAGGCATTACAGAAACCGAATTTAACATCGGCTCGTCCAAATTC
AAGGTTCTCGACGCTGGTGGGCAGCGTTCTGAACGTAAGAAGTGG
ATTCATTGTTTCGAAGGAATTACAGCAGTTTTATTTGTTTTAGCA
ATGAGTGAATACGACCAGATGTTGTTTGAGGATGAAAGAGTGAAC
AGAATGCATGAATCAATAATGCTATTTGACACGTTATTGAACTCT
AAGTGGTTCAAAGATACACCGTTTATTTTGTTTTTAAATAAAATT
GATTTGTTCGAGGAAAAGGTAAAAAGCATGCCCATAAGAAAGTAC
TTTCCTGATTACCAGGGACGTGTCGGCGATGCAGAAGCGGGTCTA
AAATATTTTGAGAAGATATTTTTGAGCTTGAATAAGACAAACAAA
CCAATCTACGTGAAACGAACCTGCGCTACCGATACCCAAACTATG
AAGTTCGTATTGAGTGCAGTCACCGATCTAATCATCCAGCAAAAC
CTTAAAAAAATTGGTATTATATGA.

Disclosed herein are biosensing compositions for detecting a target molecule in a subject, comprising a plurality of engineered yeast cells comprising a modified GPA1 protein, or a polynucleotide comprising a nucleotide sequence encoding a modified GPA1 protein, wherein said modified GPA1 protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10 (i.e., amino acid sequence of native GPA1 protein S. cerevisiae (NCBI Accession No. NP_011868.1). In some instances, the modified GPA1 protein comprises an amino acid sequence corresponding with residues 1-467 of SEQ ID NO: 10. The modified GPA1 protein can comprise one or more than one amino acid substitution compared to SEQ ID NO: 10, wherein said one or more than one amino acid substitution is selected from the following: K468E, 1469Y, G470N, 1471L, and 1472V. In some instances, the modified GPA1 protein comprises the following amino acid substitutions compared to SEQ ID NO: 10: K468E, 1469Y, G470N, 1471L, and 1472V.

Disclosed herein are biosensing compositions for detecting a target molecule in a subject, comprising a plurality of engineered yeast cells, comprising: a. a molecule responsive element comprising binding domain that interacts with said target molecule, a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, or a polynucleotide comprising a nucleotide sequence encoding a modified GPA1 protein, wherein said modified GPA1 protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10, and one or more than one amino acid substitution compared to SEQ ID NO: 10, wherein said one or more than one amino acid substitution are selected from the following: K468E, I469Y, G470N, I471L, and I472V. In some instances, the modified GPA1 protein comprises an amino acid sequence having at least 95%, at least 96%, at least 97%, at least 98%, or at least 99% sequence identity to the amino acid sequence set forth as SEQ ID NO: 10, and one or more than one amino acid substitution compared to SEQ ID NO: 10, wherein said one or more than one amino acid substitution are selected from the following: K468E, 1469Y, G470N, 1471L, and 1472V.

Disclosed herein are biosensing compositions for detecting a target molecule in a subject, comprising a plurality of engineered yeast cells, comprising: a. a molecule responsive element comprising binding domain that interacts with said target molecule, a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, or a polynucleotide comprising a nucleotide sequence encoding a modified GPA1 protein, wherein said modified GPA1 protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10, and comprises the following amino acid substitutions: K468E, I469Y, G470N, I471L, and I472V, compared to SEQ ID NO: 10.

The molecule responsive elements described herein, when present in a microorganism, such as a probiotic cell, or an engineered yeast cell, and upon interaction with a target molecule, subsequently activate and/or increase production and/or secretion of an at least one response factor (i.e., response factor) in the cell. In certain embodiments, the at least one response factor, is released, or secreted, from an engineered yeast cell into the environment. The environment can comprise a host tissue, such as a human host tissue. In certain embodiments, the environment comprises the gastrointestinal tract of a subject, such as a mammalian subject, including a human. In certain embodiments, the environment comprises the skin of a subject, such as a mammalian subject, including a human. In some embodiments, the at least one response factor is extracellular and surface-associated with an engineered yeast.

The molecule responsive elements disclosed herein, when present in an engineered yeast cell, is positioned, either inherently or by association, with a membrane protein, at the cell surface exposed to the extracellular environment. In certain embodiments, the molecule responsive element comprises a membrane protein or a transmembrane protein. In certain embodiments, the molecule responsive element is a protein. In certain embodiment, the molecule responsive element is a naturally occurring (native) protein, or a fragment, or portion or variant thereof. In certain embodiments, the molecule responsive element is a portion of a naturally occurring protein comprised in a fusion protein with one or more heterologous proteins. In certain embodiments, the molecule responsive element is a mutated version of a naturally occurring protein. In certain embodiments, the molecule responsive element is a synthetic protein. In certain embodiments, the molecule responsive element is a partly-synthetic protein. In certain embodiments, the molecule responsive element comprises one or more non-protein element. In certain embodiments, the molecule responsive element is a non-protein molecule. The molecule responsive element may be comprised of a single element or may be comprised of a plurality of elements/subunits.

In certain non-limiting embodiments, an engineered yeast cell comprises a molecule responsive element that interacts with or binds to a target molecule, wherein the molecule responsive element is coupled to an response factor, or a polynucleotide encoding an response factor, such that when the target molecule interacts with or binds to the molecule responsive element, response factor production or gene expression is increased or induced. In certain embodiments, the molecule responsive element is coupled to an response factor, or a polynucleotide encoding an response factor, such that when a target molecule interacts with or binds to the molecule responsive element, response factor production or gene expression is inhibited (for example, by binding of a transcriptional repressor).

As used herein, the term “coupled to” means that interaction of a target molecule with a molecule responsive element is causally linked, directly or indirectly, to and triggers production and secretion of an response factor that directly or indirectly modulates the target molecule in a subject. In certain embodiments, the detection event is expression of a detectable reporter gene. The molecule responsive element may be linked to expression level of the response factor through, for example, a pathway of interacting molecules (e.g., a signaling transduction pathway). This pathway may be endogenous to the engineered yeast cell, or engineered from a pathway that is endogenous to the engineered yeast cell, or engineered from a pathway that is endogenous to a species other than the engineered yeast cell. In certain embodiments, the molecule responsive element and/or signaling pathway comprises elements that are heterologous to the engineered yeast cell, and/or are from a species other than the engineered yeast cell, such as a different yeast species, or a mammalian species, such as a human. In some instances, the molecule responsive element comprises a component from or derived from a human molecule. In some instances, the molecule responsive element and/or signaling pathway comprises components derived from more than one species. For instance, the components can be derived from a yeast and a mammal. In some instances, the components can be derived from a yeast and a human.

In certain embodiments, the engineered yeast cell is engineered to express the molecule responsive element, for example, by the introduction of a nucleic acid molecule comprising a polynucleotide encoding the molecule responsive element. In certain embodiments, the nucleic acid molecule comprises a polynucleotide sequence comprising a promoter element. In certain embodiments, the polynucleotide encoding the response factor is operably linked to a polynucleotide encoding a promoter element. In certain embodiments, the promoter element is constitutively active. In certain embodiments, the promoter element is inducible. In certain embodiments, the molecule responsive element is expressed on the surface of the engineered yeast cell. In certain embodiments, the molecule responsive element is expressed on internal membranes of the engineered yeast cell. In certain embodiments, the molecule responsive element is expressed in the cytoplasm of the engineered yeast cell.

In certain embodiments, the target molecule is a natural (cognate) ligand of the molecule responsive element; the coupled target molecule-molecule responsive element (e.g., molecule responsive element bound to the target molecule) utilizes a molecule responsive element and its natural (cognate) ligand as the target molecule. In certain embodiments, the coupled analyte-receptor system is a molecule responsive element engineered to bind a different non-cognate target molecule.

One aspect of the molecule responsive element involves the increase in expression of a gene encoding for, or production and/or secretion of at least one response factor, upon interaction of the molecule responsive element with the target molecule. The molecule responsive element can comprise a signaling pathway that couples, or causally links, the interaction of a target molecule with the binding domain of the molecule responsive element to expression of a gene encoding for, or production and/or secretion of at least one response factor in the microorganism, such as the probiotic or engineered yeast cell. The cortisol responsive elements of the present disclosure can be utilized to activate gene expression and produce a response factor in the presence of cortisol. Because GC receptor functions as both a ligand binder and a transcription factor, the response factor can be engineered to be under the control of the GC receptor that is bound by the target molecule.

When the molecule responsive element comprises a human glucocorticosteroid (GC) receptor, upon interaction of said GC receptor with cortisol, said GC receptor can localize to the cell nucleus and activate gene expression. The GC receptor can activate gene expression by directly interacting with an activation domain such as a promoter that is operably linked to a gene encoding sequence. In some instances of the biosensing compositions disclosed herein, wherein the molecule responsive element comprises a human GC receptor, said GC receptor interacts with or binds to cortisol and interacts with or binds to a GC receptor activated promoter (e.g., human glucocorticosteroid response element (HRE)) operably linked to a polynucleotide sequence encoding a response factor, thereby increasing expression and production of the response factor in a yeast cell.

When the molecule responsive element comprises a human histamine receptor, or a binding domain thereof, said human receptor, or human derived receptor, which can comprise a GPCR derived protein that is bound to the membrane (e.g., transmembrane protein) will need to transmit a signal to the cell nucleus following interaction of a target molecule to activate gene expression of a response factor. In some embodiments, of the biosensing compositions disclosed herein, wherein the molecule responsive element comprises a human GPCR protein, or a fragment or variant thereof, said molecule responsive element utilizing a native yeast signaling pathway with the yeast cell to activate gene expression. For instance, the molecule responsive element can be engineered to interact with a native yeast signaling pathway in order to activate gene expression of the response factor in the yeast cell. In some instances, a molecule responsive element that comprises a human component can further comprise a polypeptide sequence comprising a yeast signaling polypeptide.

The molecule responsive element can comprise a fusion protein comprising any GPCR, or a functional fragment or variant thereof. For instance, the fusion protein can comprise a naturally, occurring GPCR, or can comprise a portion of any naturally, occurring GPCR. In some instances, the molecule responsive element comprises a GPCR protein, or a functional fragment or variant thereof, that comprises a histamine receptor.

The biosensing compositions for detecting a target molecule in a subject comprise a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain.

The biosensing compositions for detecting a target molecule in a subject comprise a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said polynucleotide encoding a response factor is operably fused to an activation domain comprising a Ste-12-activated promoter.

The biosensing compositions for detecting a target molecule in a subject comprise a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said polynucleotide encoding a response factor is operably fused to an activation domain comprising a Ste-12-activated promoter, wherein said engineered yeast cells comprise at least one genomic modification in at least one yeast cell. In some instances, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of said engineered yeast within said plurality comprise at least one genomic modification. A “genomic modification” as used herein in the context of engineered yeast refers to any alteration of the genomic sequence that differs from a wild-type yeast cell. For example, a genomic modification can include a substitution, deletion and/or insertion of at least one nucleotide within the genome of a yeast cell compared to a wild-type yeast cell. In some instances, a gene encoding sequence is deleted. More than one gene encoding sequence can be removed from the genome. In some instances, a gene encoding sequence is disrupted, such that the gene does not encode its original polypeptide.

The engineered yeast cells disclosed herein comprising a molecule responsive element comprising a recombinant G-protein coupled receptor (GPCR) fusion protein comprising a histamine binding domain can have at least one gene on the genome deleted and/or disrupted, such that the polypeptide encoding by said at least one gene is not produced or is not functional. A polynucleotide encoding a molecule responsive element comprising a recombinant G-protein coupled receptor (GPCR) fusion protein can be codon-optimized for expression in yeast, such as Saccharomyces cerevisiae and/or Saccharomyces boulardii.

In some instances, the engineered yeast cells have at least one gene involved in native G-protein coupled receptor (GPCR) signaling deleted. The deletion of at least one gene involved in native G-protein coupled receptor signaling is important to minimize native yeast GPCR signaling that may increase gene expression, or production and/or secretion, of the response factor. The deleted gene can STE2, FAR1, SST2, MAT-A, or any combination thereof. AN engineered yeast can comprise a mutation and/or deletion in at least one of the following genes: STE2, FAR1, SST2, and MTA-A. An engineered yeast can comprise a mutation and/or deletion in two of the following genes: STE2, FAR1, SST2, and MTA-A. An engineered yeast can comprise a mutation and/or deletion in three of the following genes: STE2, FAR1, SST2, and MTA-A. An engineered yeast can comprise a mutation and/or deletion in all of the following genes: STE2, FAR1, SST2, and MTA-A. STE2 is a gene in the yeast Saccharomyces cerevisiae that encodes the ligand-binding component of the alpha-factor receptor. The STE2 gene encodes a seven transmembrane domain receptor that transmits signals through a heterotrimeric G protein; thus, it is considered class D G-protein coupled receptors (GPCR). The FAR1 gene encodes a protein crucial for cell cycle arrest during mating, essentially acting as a “factor arrest” by inhibiting a cyclin-dependent kinase, and is also required for proper cell polarization in response to mating pheromones; meaning it plays a key role in orienting the yeast cell towards its mating partner when exposed to pheromones. SST2 encodes a protein that functions to desensitize the signal response to pheromones, and is associated with G protein signaling. MAT-A encodes a protein that controls the expression of genes involved in the pheromone signaling pathway triggered by the G protein. The engineered yeast cell can comprise a full deletion of the STE2 gene. The engineered yeast cell can comprise any genome modification that disrupts production of a functional STE2 gene product. The engineered yeast cell can comprise a full deletion of the FAR1 gene. The engineered yeast cell can comprise any genome modification that disrupts production of a functional FAR1 gene product. The engineered yeast cell can comprise a full deletion of the SST2 gene. The engineered yeast cell can comprise any genome modification that disrupts production of a functional SST2 gene product. The engineered yeast cell can comprise a full deletion of the MAT-A gene. The engineered yeast cell can comprise any genome modification that disrupts production of a functional MAT-A gene product.

Disclosed herein are biosensing compositions for detecting a target molecule in a subject, comprising a plurality of engineered yeast cells, comprising: a. a molecule responsive element comprising binding domain that interacts with said target molecule, a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, or a polynucleotide comprising a nucleotide sequence encoding a modified GPA1 protein, wherein said modified GPA1 protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10, and one or more than one amino acid substitution compared to SEQ ID NO: 10, wherein said one or more than one amino acid substitution are selected from the following: K468E, I469Y, G470N, I471L, and I472V, wherein the plurality of engineered yeast cells comprise one or more than one genome modification that disrupts production of a functional STE2, FAR1, SST2, and/or MTA-A gene product. In some instances, the engineered yeast comprises one or more than one genomic modification that disrupts productions of a functional gene product encoded from one or more than one of the following genes: STE2, FAR1, SST2, and MTA-A. In some instances, the engineered yeast comprises one or more than one genomic modification that disrupts productions of a functional gene product encoded from the following genes: STE2, FAR1, SST2, and MTA-A. In some instances, the engineered yeast comprises a full deletion of the following genes: STE2, FAR1, SST2, and MTA-A.

Disclosed herein are biosensing compositions for detecting a target molecule in a subject, comprising a plurality of engineered yeast cells, comprising: a. a molecule responsive element comprising binding domain that interacts with said target molecule, a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, or a polynucleotide comprising a nucleotide sequence encoding a modified GPA1 protein, wherein said modified GPA1 protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10, and comprises the following amino acid substitutions: K468E, I469Y, G470N, I471L, and I472V, compared to SEQ ID NO: 10, wherein the plurality of engineered yeast cells comprise one or more than one genome modification that disrupts production of a functional STE2, FAR1, SST2, and/or MTA-A gene product. In some instances, the engineered can yeast comprise one or more than one genomic modification that disrupts productions of a functional gene product encoded from one or more than one of the following genes: STE2, FAR1, SST2, and MTA-A. In some instances, the engineered yeast comprises one or more than one genomic modification that disrupts productions of a functional gene product encoded from the following genes: STE2, FAR1, SST2, and MTA-A. In some instances, the engineered yeast comprises a full deletion of the following genes: STE2, FAR1, SST2, and MTA-A.

Disclosed herein are biosensing compositions for detecting a target molecule in a subject, comprising a plurality of engineered yeast cells, comprising: a. a molecule responsive element comprising binding domain that interacts with said target molecule, or a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, or a polynucleotide comprising a nucleotide sequence encoding a modified GPA1 protein, wherein said modified GPA1 protein comprises an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8, wherein the plurality of engineered yeast cells comprise one or more than one genome modification that disrupts production of a functional STE2, FAR1, SST2, and/or MTA-A gene product. In some instances, the engineered can yeast comprise one or more than one genomic modification that disrupts productions of a functional gene product encoded from one or more than one of the following genes: STE2, FAR1, SST2, and MTA-A. In some instances, the engineered yeast comprises one or more than one genomic modification that disrupts productions of a functional gene product encoded from the following genes: STE2, FAR1, SST2, and MTA-A. In some instances, the engineered yeast comprises a full deletion of the following genes: STE2, FAR1, SST2, and MTA-A.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, wherein said modified GPA1 protein comprises an amino acid sequence set forth as SEQ ID NO: 8, wherein the plurality of engineered yeast comprises a full deletion of the following genes: STE2, FAR1, SST2, and MTA-A.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, wherein said modified GPA1 protein comprises an amino acid sequence set forth as SEQ ID NO: 8, wherein the plurality of engineered yeast comprises a full deletion of the following genes: STE2, FAR1, SST2, and MTA-A, wherein the yeast cells comprise Saccharomyces.

Disclosed herein are biosensing compositions for detecting a target molecule in a subject, comprising a plurality of engineered yeast cells, comprising: a. a molecule responsive element comprising binding domain that interacts with said target molecule; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said activation domain comprises a nucleotide sequence set forth as SEQ ID NO: 12, and wherein said plurality of engineered yeast cells further comprises: c. a modified GPA1 protein, wherein said modified GPA1 protein comprises an amino acid sequence set forth as SEQ ID NO: 8, wherein the plurality of engineered yeast comprises a full deletion of the following genes: STE2, FAR1, SST2, and MTA-A, wherein the yeast cells comprise Saccharomyces.

When a molecule responsive element comprises a human GPCR protein comprising a yeast intracellular signaling domain, said molecule responsive element can activate gene expression of the response factor through a native yeast signaling pathway within the engineered yeast cell. In certain embodiments, a component of a yeast pheromone sensing system is incorporated into the molecule responsive element and/or signaling transduction pathway in yeast to activate gene expression in the yeast cell. The yeast pheromone sensing system can comprise any yeast pheromone sensing system. Any yeast GPCR protein can be used. For instance, the molecule responsive element can comprise a portion of any yeast GPCR protein. In such instances, the molecule responsive element comprises and/or interacts with a yeast pheromone sensing system or signaling pathway comprising components derived from a yeast pheromone sensing system. The yeast pheromone signaling pathway is well studied structurally and is functionally similar to hormone and neurotransmitter signaling pathways in mammals, as described in Wang, Y. & Dohlman, H. G. Pheromone signaling mechanisms in yeast: a prototypical sex machine. Science 306, 1508-1509 (2004), the contents of which are incorporated herein in their entirety.

In certain embodiments, the molecule responsive element comprises a polypeptide comprising an amino acid sequence encoding a yeast Ste2 receptor or Ste3 receptor, or a fragment or derivative thereof, wherein the molecule responsive element is modified such that it interacts with or binds to the target molecule rather than a yeast pheromone. For instance, a binding domain that interacts with the target molecule other than a yeast pheromone (e.g., cortisol and/or histamine) can be synthetically introduced into the Ste2 receptor, Ste3 receptor, or derivative receptor thereof. In certain embodiments, the molecule responsive element comprises a receptor or portion thereof comprising a polypeptide comprising an amino acid sequence having at least about 40%, at least about 50%, at least about 60%, at least about 70%, at least about 80%, at least about 85%, at least about 90%, at least about 95%, at least about 98%, at least about 99% or greater sequence identity to a native yeast Ste2 or yeast Ste3 receptor.

In certain embodiments the molecule responsive element comprises a receptor comprising a yeast GPCR polypeptide, or a fragment or variant thereof. In certain embodiments, the GPCR is a fungal GPCR. In certain embodiments, the GPCR is a fungal pheromone GPCR. In certain non-limiting embodiments, a portion of a fungal Ste2-type or Ste3-type GPCR is comprised within the molecule responsive element. While any peptide-sensing GPCR can be repurposed as a detection element in a yeast cell, fungal pheromone GPCRs have several key advantages for biosensor engineering. First, this type of GPCRs (GPCRs homologous to the S. cerevisiae Ste2) couple robustly to the host/native pheromone pathway, and several have been expressly validated in S. cerevisiae with little to no further modifications. Second, fungal pheromone GPCRs from related fungi recognize different peptides based on the natural evolution of this class of GPCR. For example, fungal GPCRs recognize a diverse set of peptide ligands. Third, fungal pheromone GPCRs are highly specific for their respective peptides, since they must mediate the species-specific mating reaction while preventing interspecies breeding. Furthermore, though there is no crystal structure of these GPCRs, extensive biochemical characterization and mutagenesis data indicates that the S. cerevisiae GPCR has a large binding interface across the seven transmembrane helices and the extracellular loops modulating ligand binding.

In one aspect, the present disclosure provides a yeast cell, or plurality of yeast cells, comprising a molecule responsive receptor, or a polynucleotide encoding a molecule responsive receptor, which comprises a portion of a yeast protein in order to detect a human molecule and activate gene expression of a response factor in a yeast cell. The molecule responsive factor can comprise any portion or fragment of a yeast signaling cascade polypeptide, such as an intracellular domain, that aids in signaling in the yeast cell. In certain embodiments, this modification is achieved via directed evolution. A mammalian or hybrid G-protein can be used to enhance GPCR signal transduction in a yeast cell. In another specific aspect, the present disclosure provides a yeast cell, or plurality of yeast cells, comprising a molecule responsive receptor, or a polynucleotide encoding a molecule responsive receptor, which comprises a portion of a human protein in order to detect a human molecule and activate gene expression of a response factor in a yeast cell. When a molecule responsive receptor is expressed in a yeast, the yeast can further comprise a signaling pathway component capable of interacting with the molecule responsive element and/or transmitting a signal upon binding of the molecule responsive element with a target molecule. In some instances, the yeast expressing a human receptor, or portion or fragment thereof, further comprises a recombinant signaling protein that interacts with the human receptor. The recombinant signaling protein can be a recombinant G protein, as described elsewhere herein. In some instances, the recombinant G protein is a wild-type G protein comprising at least one modification (e.g., amino acid substitution, insertion, and/or deletion). Any amino acid modification to a yeast G protein can be made to enable it to interact with and initiate a signaling pathway signal from a human receptor. In some instances, the recombinant G protein activates a native signaling pathway found in yeast. In such instances, it would be readily understood by a person having skill in the art that the recombinant G protein be capable of initiate a native yeast signaling transduction pathway of a native and/or recombinant GPCR within the yeast.

G protein-coupled receptors (GPCRs), also known as seven-(pass)-transmembrane domain receptors, 7TM receptors, heptahelical receptors, serpentine receptors, and G protein-linked receptors (GPLR), form a large group of evolutionarily related proteins that are cell surface receptors that detect molecules outside the cell and activate cellular responses. They are coupled with G proteins. They pass through the cell membrane seven times in the form of six loops (three extracellular loops interacting with ligand molecules, three intracellular loops interacting with G proteins, an N-terminal extracellular region and a C-terminal intracellular region) of amino acid residues, which is why they are sometimes referred to as seven-transmembrane receptors (see Zhang, Jian V.; Li, Lei; Huang, Qingsheng; Ren, Pei-Gen (1 Jan. 2013). “Chapter Three—Obestatin Receptor in Energy Homeostasis and Obesity Pathogenesis”. In Tao, Ya-Xiong (ed.).

Progress in Molecular Biology and Translational Science. G Protein-Coupled Receptors in Energy Homeostasis and Obesity Pathogenesis. Vol. 114. Academic Press. pp. 89-107; and Trzaskowski B, Latek D, Yuan S, Ghoshdastider U, Debinski A, Filipek S (2012). “Action of molecular switches in GPCRs—theoretical and experimental studies”. Current Medicinal Chemistry. 19 (8): 1090-109). Ligands can bind either to the extracellular N-terminus and loops (e.g. glutamate receptors) or to the binding site within transmembrane helices (rhodopsin-like family). GPCRs are generally activated by agonists, although a spontaneous auto-activation of an empty receptor has also been observed. G protein-coupled receptors are thought to be present in only eukaryotes, including yeast, and choanoflagellates, as described in King N, Hittinger C T, Carroll S B (July 2003). “Evolution of key cell signaling and adhesion protein families predates animal origins”. Science. 301 (5631): 361-3, the contents of which are incorporated herein in their entirety. The ligands that bind and activate these receptors include light-sensitive compounds, odors, pheromones, hormones, and neurotransmitters, and vary in size from small molecules to peptides to large proteins. G protein-coupled receptors are involved in many diseases.

There are two principal signal transduction pathways involving the G protein-coupled receptors: the i) cAMP signal pathway and ii) phosphatidylinositol signal pathway, as described in Gilman A G (1987). “G proteins: transducers of receptor-generated signals”. Annual Review of Biochemistry. 56 (1): 615-49, the contents of which are incorporated herein in their entirety.

When a ligand interacts with binds to the GPCR it causes a conformational change in the GPCR, which allows it to act as a guanine nucleotide exchange factor (GEF). The GPCR can then activate an associated G protein by exchanging the GDP bound to the G protein for a GTP. The G protein's a subunit, together with the bound GTP, can then dissociate from the β and γ subunits to further affect intracellular signaling proteins or target functional proteins directly depending on the a subunit type (Gαs, Gαi/o, Gαq/11, Gα12/13), as described in Wettschureck N, Offermanns S (October 2005). “Mammalian G proteins and their cell type specific functions”. Physiological Reviews. 85 (4): 1159-204, the contents of which are incorporated herein in their entirety. In some embodiments, the engineered yeast encodes a recombinant G protein a subunit, β subunit, and/or γ subunit, wherein the subunit differs from a wild-type yeast subunit by at least one amino acid modification (i.e., modified G protein subunit). It would be understood by a skilled artisan that the recombinant G protein subunit retains the ability to mediate intracellular signaling. The engineered yeast can encode a modified G protein a subunit, whereby the G protein is capable of interacting with a human GPCR and transmitting a signal within the yeast cell to activate expression of a polynucleotide encoding a response factor. The engineered yeast can encode β modified G protein a subunit, whereby the G protein is capable of interacting with a human GPCR and transmitting a signal within the yeast cell to activate expression of a polynucleotide encoding a response factor. The engineered yeast can encode a modified G protein γ subunit, whereby the G protein is capable of interacting with a human GPCR and transmitting a signal within the yeast cell to activate expression of a polynucleotide encoding a response factor. In some instances, when the yeast encodes a modified G protein α subunit, the α subunit retains the ability to bind GTP.

G proteins, also known as guanine nucleotide-binding proteins, are a family of proteins that act as molecular switches inside cells, and are involved in transmitting signals from a variety of stimuli outside a cell to its interior. Their activity is regulated by factors that control their ability to bind to and hydrolyze guanosine triphosphate (GTP) to guanosine diphosphate (GDP). When they are bound to GTP, they are ‘on’, and, when they are bound to GDP, they are ‘off’. G proteins belong to the larger group of enzymes called GTPases.

There are two classes of G proteins. The first function as monomeric small GTPases (small G-proteins), while the second function as heterotrimeric G protein complexes (see Hurowitz E H, Melnyk J M, Chen Y J, Kouros-Mehr H, Simon M I, Shizuya H (April 2000). “Genomic characterization of the human heterotrimeric G protein alpha, beta, and gamma subunit genes”. DNA Research. 7 (2): 111-20). The latter class of complexes is made up of alpha (Ga), beta (GO) and gamma (Gy) subunits. In addition, the beta and gamma subunits can form a stable dimeric complex referred to as the beta-gamma complex (see Clapham D E, Neer E J (1997). “G protein beta gamma subunits”. Annual Review of Pharmacology and Toxicology. 37: 167-203).

Heterotrimeric G proteins located within the cell are activated by G protein-coupled receptors (GPCRs) that span the cell membrane. Upon binding of a signaling molecule to a domain of the GPCR located outside the cell (e.g., GPCR ectodomain), an intracellular GPCR domain subsequently activates a G protein. Some active-state GPCRs have also been shown to be “pre-coupled”with G proteins, whereas in other cases a collision coupling mechanism is thought to occur. The G protein triggers a cascade of further signaling events that finally results in a change in cell function. G protein-coupled receptors and G proteins working together transmit signals from many hormones, neurotransmitters, and other signaling factors. G proteins regulate metabolic enzymes, ion channels, transporter proteins, and other parts of the cell machinery, controlling transcription, motility, contractility, and secretion, which in turn regulate diverse systemic functions such as embryonic development, learning and memory, and homeostasis (see Neves S R, Ram P T, Iyengar R (May 2002). “G protein pathways”. Science. 296 (5573): 1636-9).

GPCRs bind to their natural ligands and induce a cascade of MAP kinase signaling, which can ultimately lead to transcriptional activation of genes under control of the GPCR signaling pathway. In humans, histamine is a natural ligand for human GPCRs, which can activate GPCR signaling in human cells. In some embodiments, the histamine binding domains of the present disclosure comprises a histamine binding domain of a human GPCRs. In some embodiments, the histamine binding domain is derived from a human GPCR.

In some embodiments, interaction of histamine with the histamine binding domain of the responsive element of the present disclosure results in activation of the GPCR signaling cascade. In certain non-limiting embodiments, the present disclosure provides an engineered yeast that uses G-protein coupled receptors (GPCRs) to detect a range of target molecules, or fragments thereof, and subsequently produces a response factor in response. In certain non-limiting embodiments, the present disclosure provides methods of engineering target molecule-activated GPCRs to detect non-cognate or cognate target molecules. The binding domain of the molecule responsive element of the present disclosure can comprise a protein, such as a recombinant protein. The recombinant protein can comprise a recombinant, fusion protein and/or a transmembrane protein. In some embodiments, the transmembrane protein comprises a recombinant G-protein coupled receptor (GPCR) fusion protein. The recombinant GPCR fusion protein can be engineered to comprise a histamine binding domain. The histamine binding domain can be synthetically introduced into the GPCR protein. In some instances, a GPCR fusion protein can comprise a synthetic histamine binding domain comprising a histamine binding ectodomain. In some embodiments, the GPCR fusion protein comprises an intracellular signaling domain. The GPCR fusion protein can comprise a histamine binding ectodomain and a signaling intracellular domain. The histamine binding ectodomain can be from a human histamine binding protein. The signaling intracellular domain can be from a yeast signaling protein, such as a yeast GPCRs.

The engineered yeast cell can comprise a molecule responsive element which is coupled to a nucleic acid molecule comprising a polynucleotide encoding a response factor, by a G-protein signaling pathway. In such instances, the molecule responsive element comprises a molecule responsive element comprising a polypeptide comprising an amino acid sequence encoding a human G-protein coupled receptor (GPCR) or a fragment or variant thereof having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a human GPCR. In some embodiments, the human GPCR comprises a histamine binding domain of a human GPCR, and an intracellular signaling domain of a yeast intracellular domain protein, or a fragment or variant thereof having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a yeast intracellular signaling domain. In certain embodiments, the molecule responsive element comprises a receptor comprising a non-native and/or recombinant GPCR receptor.

The molecule responsive elements disclosed herein are capable of interacting with a target molecule across a range of concentrations with the strength of the interaction often increasing as the concentration of the target molecule increases. The molecule responsive elements can be designed to have specific binding affinities, resulting in varying degrees of sensitivity to the target molecule. This means that the concentration at which a significant response is elicited can be modulated by engineering the binding domain and the associated signaling pathways. The concentration threshold at which a noticeable response occurs is specific to each molecule responsive element and can be adjusted to match physiological levels relevant to the target molecule. For instance, two molecule responsive elements that interact with the same target molecule can differ in their sensitivity and the concentration range over which they respond, depending on their binding affinities and signaling dynamics. The interaction of the molecule responsive element with a target molecule is dependent upon the concentration of the target molecule within the surrounding environment, such as the gastrointestinal tract of the subject. The term “sensitivity” can refer to the ability of the molecule responsive element to respond to changes in the concentration of the target molecule, including the minimal concentration required to elicit a detectable response and the steepness of the response curve. The molecule responsive elements can be engineered to have varying sensitivities by modifying the binding domain or the signaling cascade to achieve the desired response profile.

Moreover, the molecule responsive elements disclosed herein can comprise signaling cascades that amplify the response upon interaction with the target molecule. By incorporating mechanisms such as cooperative binding or feedback loops within the signaling pathway, the system can exhibit threshold-like behavior, where a significant response is activated when the target molecule concentration reaches a certain level. This allows for more precise control over the activation of the response factor production in the engineered yeast cells.

In some instances, the molecule responsive element comprises a binding domain that interacts with or binds to cortisol, or a variant thereof, or a molecule comprising cortisol. When the target molecule is cortisol, the threshold concentration can be at least 0.1 nM, at least 0.5 nM, at least 1 nM, at least 5 nM, at least 10 nM, at least 100 nM, at least 500 nM, at least 1 μM, or at least 5 μM. In some instances, the threshold concentration for binding cortisol is less than 0.1 nM. In some instances, the threshold concentration for binding cortisol is more than 5 μM. Disclosed herein are biosensing compositions for detecting a target molecule in a subject, wherein the composition comprises a plurality of engineered yeast cells comprising a molecule responsive element comprising a binding domain that interacts with or binds to cortisol, and a nucleic acid molecule comprising a polynucleotide encoding a response factor, wherein said binding domain interacts with or binds to cortisol when cortisol is present at a concentration of at least 0.1 nM, at least 0.5 nM, at least 1 nM, at least 5 nM, at least 10 nM, at least 100 nM, at least 500 nM, at least 1 μM, or at least 5 μM.

In some instances, the molecule responsive element comprises a binding domain that binds to histamine, or a variant thereof, or a molecule comprising histamine. When the target molecule is histamine, the threshold concentration can be at least 0.1 nM, at least 0.5 nM, at least 1 nM, at least 5 nM, at least 10 nM, at least 100 nM, at least 500 nM, at least 1 μM, or at least 5 μM. In some instances, the threshold concentration for binding histamine is less than 0.1 nM. In some instances, the threshold concentration for binding histamine is more than 5 μM. Disclosed herein are biosensing compositions for detecting a target molecule in a subject, wherein the composition comprises a plurality of engineered yeast cells comprising a molecule responsive element comprising a binding domain that interacts with or binds to histamine, and a nucleic acid molecule comprising a polynucleotide encoding a response factor, wherein said binding domain interacts with or binds to histamine when histamine is present at a concentration of at least 0.1 nM, at least 0.5 nM, at least 1 nM, at least 5 nM, at least 10 nM, at least 100 nM, at least 500 nM, at least 1 μM, or at least 5 μM.

The engineered yeast cells disclosed herein comprising the molecule responsive element are designed to detect the presence of a target molecule in vivo in a subject. Upon detection of a target molecule, the engineered yeast cell can produce a response factor capable of modulating the level or activity of the target molecule or the level or activity of a molecule other than the target molecule. The target molecules that are to be detected in vivo can be detected at any concentration. In some instances, it may be desirable to release a response factor only when the target molecule is present at or above a threshold concentration. For instance, the target molecule may be a molecule that is present on the skin or within the gastrointestinal tract of a subject, wherein a threshold concentration of the target molecule is associated with a condition, a disease, or a sign or symptom of disease. In such instances, wherein a threshold concentration of a target molecule is associated with a condition, an ailment, a disease, or a sign or symptom of a condition, an ailment, or a disease, it is desirable to produce and release a response factor only when the target molecule is present at or above a threshold concentration. It some instances, it may be desired to reduce the levels of the target molecule. The level of the target molecule can be reduced by competitive inhibition, degradation, signal interference, or other means known in the art. In such instances, the levels of the target molecule may be reduced by a percentage of the detected concentration. In some embodiments, it may not be desired to completely remove the target molecule. In some instances, it may be desired to limit the activity, such as the enzymatic activity of the target molecule, rather than reduce the levels of the target molecule. In such instances, the engineered yeast cell can be used to modulate the target molecule only if and/or when the target molecule is present above the threshold concentration. In such instances, the engineered yeast will not produce and release a response factor capable of modulating the target molecule when the target molecule is present below the threshold concentration.

Interaction of a target molecule with the molecule responsive element triggers the production and/or secretion of a response factor. Said interaction can be associated with an increase in expression of a gene encoding the response factor within the engineered yeast cell. The response factor that is produced upon interaction of the target molecule is specific to the target molecule. For example, response factors for cortisol as a target molecule could be 11β-hydroxysteroid dehydrogenase Type 2 (11β-HSD2) for the enzymatic degradation of cortisol, corticosteroid-binding globulin (CBG), short-chain fatty acids (SCFAs), such as butyrate, Glucocorticoid Receptor Antagonists (e.g., RU-486 Mimetics), bile salt hydrolases and/or target bile acids thereof (e.g., deconjugated bile acids), Gamma-Aminobutyric Acid (GABA), Serotonin/Melatonin and their Precursors (e.g., tryptophan, 5-Hydroxytryptophan, 5-HTP), Peptide YY (PYY), Antioxidant Enzymes (e.g., Superoxide Dismutase, SOD), Heat Shock Proteins (e.g., HSP70), glucocorticoid receptor antagonists (e.g., RU-486 mimetics) Exopolysaccharides, Microbial Anti-stress Molecules (e.g., Indole-3-Lactic Acid), L-theanine, Anandamide, Nicotinamide, Acetylcholine, Valerenic Acid, and/or Beta-Caryophyllene. A response factor for histamine could include: Histamine Oxidase (Diamine Oxidase, DAO), Histamine N-Methyltransferase (HNMT), Peptide-based Histamine Receptor Antagonists (against H1 receptor for allergic symptom, H2 receptor for gastric symptoms (ulcers, reflux), potentially against H3 and H4 receptors as well), synthetic and/or recombinant histamine or histamine receptor-binding molecules (e.g., peptides, scFvs, ankyrin repeat proteins, DNA/RNA molecules), Anti-Inflammatory and Barrier-Protective Molecules: SCFA/butyrate, Glutathione to neutralize reactive oxygen species and reduce oxidative damage induced by histamine-mediated inflammatory responses, Serine Protease Inhibitors (e.g., alpha-1 antitrypsin), polyphenols, and/or S-Adenosylmethionine (SAM-e).

In certain embodiments, the response factor that is released serves to modulate the target molecule. The response factors described herein are specific to the target molecule. The term “modulate” as used herein in regard to the target molecule refers to a change in the structure or function of the target molecule. In certain embodiments, the release of the response factor into the environment can decrease the amount, or the levels, of the target molecule. In such instances, the response factor may bind and degrade the target molecule. In certain embodiments, the response factor can comprise a nuclease or protease. In some embodiments, the response factor can comprise a small molecule inhibitor or small molecule degrader, such as a small molecule degrading enzyme.

In certain embodiments, the release of the response factor into the environment can decrease the functionality of the target molecule. In certain embodiments, wherein the response factor reduces the activity or functionality of the target molecule, the response factor can recognize and bind the target molecule, inhibiting the ability of the target molecule to bind molecules, such as receptors or cells in a subject, other than the response factor. In such instances, the response factor serves to “quench” the undesired binding of the target molecule within the body. In some instances, the response factor exerts its influence on the levels or the activity of the target molecule indirectly, such as through a mechanism other than direct binding. For instance, the response factor may interact with or bind to an intermediate molecule, wherein the intermediate molecule directly interacts with or binds to the target, such as the target molecule, itself. In some instances, the response factor or intermediate molecule can interact with a receptor for the target molecule. In some embodiments the response factor can interact directly with a receptor of the target molecule, or the intermediate can interact with the receptor of a target molecule.

In certain embodiments, the engineered yeast of the present disclosure comprising a molecule responsive element, or a polynucleotide comprising a nucleotide sequence encoding the molecule responsive element, further comprises a polynucleotide encoding a response factor. In such instances, interaction of a target molecule with the binding domain of the molecule responsive element increases gene expression from the polynucleotide encoding the response factor. In some instances, the molecule responsive element, upon interacting with the target molecule, directly interacts with or binds to the polynucleotide encoding the response factor to activate gene expression. In some instances, gene expression of the polynucleotide encoding the response factor is activated, or increased, by a signaling cascade that is initiated upon interaction of the molecule responsive element with the target molecule. In such instances, the signaling cascade can comprise a MAPK signaling cascade.

Interaction of a target molecule can induce or alternatively repress expression of the response factor in the engineered yeast cell. In the absence of a target molecule (i.e., no interaction of a target molecule with the molecule responsive element of the engineered yeast), there may be essentially no response factor production, or gene expression, or production or gene expression of the response factor may occur at an undetectable level, or may occur at a baseline level that detectably increases upon binding of the target molecule to the molecule responsive element.

The engineered yeast cells of the present disclosure, upon interaction of a target molecule with the binding domain of the molecule responsive element, may secrete a response factor into the environment that directly modulates the target molecule. In general, microbial cells have been used to produce therapeutic molecules such as peptides, proteins and other bioactive small-molecules (Bourbonnais et al., “Secretion of Somatostatin by Saccharomyces cerevisiae Correct Proteolytic Processing of pro-α-Factor-Somatostatin Hybrids Requires the Products of the KEX2 and STE13 Genes,” J. Biol. Chem. 263(30):15342-15347 (1988); Miyajima et al., “Secretion of mature mouse interleukin-2 by Saccharomyces cerevisiae: use of a general secretion vector containing promoter and leader sequences of the mating pheromone α-factor,” Gene 37:155-161 (1985); Ro et al., “Production of the antimalarial drug precursor artemisinic acid in engineered yeast,” Nature 440:940-943 (2006)). In certain embodiments, interaction of a target molecule with a molecule responsive element of a presently disclosed engineered yeast cell can be coupled to the biosynthesis and secretion of a response factor in a subject.

In certain embodiments, the secretion of the response factor from the engineered microorganism can occur on the skin or in the esophagus, nasal tissue, mouth, vaginal tissue, lungs, stomach, small intestines, large intestines, digestive tract, or gastrointestinal tract of said subject. In certain embodiments, the secretion from the microorganism results in release of a response factor into the gastrointestinal tract of a subject. The release of a response factor can be used to directly modulate the target molecule detected by a presently disclosed cell.

The response factor can be produced and secreted by at least one engineered yeast cell on the skin or in the esophagus, nasal tissue, mouth, vaginal tissue, lungs, stomach, small intestines, large intestines, digestive tract, or gastrointestinal tract of said subject. For instance, the response factor can be produced and secreted by at least one engineered Saccharomyces cell on the skin or in the esophagus, nasal tissue, mouth, vaginal tissue, lungs, stomach, small intestines, large intestines, digestive tract, or gastrointestinal tract of said subject. The response factor can be produced and secreted by at least one engineered Saccharomyces cerevisiae cell on the skin or in the esophagus, nasal tissue, mouth, vaginal tissue, lungs, stomach, small intestines, large intestines, digestive tract, or gastrointestinal tract of said subject. The response factor can be produced and secreted by at least one engineered Saccharomyces boulardii cell on the skin or in the esophagus, nasal tissue, mouth, vaginal tissue, lungs, stomach, small intestines, large intestines, digestive tract, or gastrointestinal tract of said subject.

In one embodiment, to enhance the robustness of target molecule detection, signal amplification strategies inspired by yeast's natural pheromone signaling mechanisms can be used. Specifically, upon binding of a target molecule, a genetically engineered system within the yeast cell activates transcription factors that upregulate the expression of enzymes responsible for synthesizing pheromone-like molecules or alternative GPCR ligands such as modified α-factor. This creates a positive feedback loop, significantly increasing the production of response factors only when the target molecule is present above a certain concentration. By integrating regulatory elements like inducible promoters and feedback inhibitors, the system ensures controlled amplification, preventing excessive response and maintaining cellular balance. The MAPK signaling cascade can be leveraged to facilitate this process, ensuring that signal amplification is tightly coupled with the desired modulation of the target molecule. This approach can mimic the natural pheromone signaling found in S. cerevisiae and other fungi, utilizing GPCRs such as Ste2 to achieve enhanced sensitivity and specificity in target detection.

### IV. Methods of Use

The present disclosure provides methods of binding, detecting, and/or reducing the levels of a target molecule in a subject in need thereof. Also disclosed are methods of reducing the levels of an intermediate molecule and/or effects of elevated target molecule within a target tissue. Also disclosed are methods of treating a disease or disorder or reducing a sign or symptom of a disease or disorder. Also disclosed are methods of maintaining a healthy biological function within a tissue of a subject. These methods comprise administering to said subject a therapeutic amount of any biosensing composition, probiotic or pharmaceutical composition disclosed herein.

In the methods of treating a disease or disorder or reducing a sign or symptom of a disease or disorder, the disease or disorder can be a stress disorder, an inflammatory disorder, an autoimmune disorder, a disorder associated with microbiome dysbiosis, a hormonal imbalance disorder, an infection, a skin disorder, and/or a gastrointestinal or digestive disorder. The disorder can be any disorder that is associated with elevated levels of a target molecule. The levels of the target molecule may be elevated for an extended period of time. Alternatively, the disorder may involve transient elevated levels of the target molecule.

The methods disclosed herein are particularly effective at reducing the levels and/or activity or effects of a target molecule that is present at a concentration above a specific threshold concentration. As described herein, the threshold concentration is specific to the target molecule, and the biosensing composition disclosed herein can be specifically designed to bind, detect, and/or reducing the levels of a target molecule in response to a specific threshold concentration. Response factors disclosed herein can interact with target molecules, or intermediate molecules that can impact target molecules, at a molecular level to result in degradation, inhibition, or sequester of target molecules, inhibit the receptor, or modulate downstream effects of the elevated target molecule. Downstream effects of elevated target molecule levels include inflammation, stress, and other conditions described elsewhere herein.

In some embodiments of the methods disclosed herein, a subject has elevated cortisol levels. In some embodiments of the methods disclosed herein, a subject has elevated histamine levels. Administration of the biosensing compositions, or probiotic or pharmaceutical composition comprising the biosensing composition, disclosed herein are effective at reducing the levels and/or activity of cortisol on the skin or in the esophagus, nasal tissue, mouth, vaginal tissue, lungs, stomach, small intestines, large intestines, digestive tract, or gastrointestinal tract of said subject. Administration of the biosensing compositions, or probiotic or pharmaceutical composition comprising the biosensing composition, disclosed herein are effective at reducing the levels and/or activity of histamine on the skin or in the esophagus, nasal tissue, mouth, vaginal tissue, lungs, stomach, small intestines, large intestines, digestive tract, or gastrointestinal tract of said subject.

In the methods of methods of maintaining a healthy biological function within a tissue of a subject, the tissue can be the skin or in the esophagus, nasal tissue, mouth, vaginal tissue, lungs, ocular tissues, stomach, small intestines, large intestines, digestive tract, or gastrointestinal tract of said subject. Maintaining a healthy biological function can involve reducing the levels and/or activity of a target molecule with a specific tissue. For instance, elevated levels of a target molecule may contribute to dysbiosis within a specific tissue, or to multiple tissues within a subject. Maintaining a healthy biological function can involve reducing the levels and/or activity of a target molecule with a specific tissue and can reduce dysbiosis in said tissue following administration of the biosensing composition by reducing the levels and/or activity of cortisol with said tissue. Maintaining a healthy biological function can involve reducing the levels and/or activity of a target molecule with a specific tissue and can reduce dysbiosis in said tissue following administration of the biosensing composition by reducing the levels and/or activity of histamine with said tissue.

Following administration of the biosensing compositions, or probiotic or pharmaceutical composition comprising the biosensing composition, disclosed herein to a subject, the molecule responsive element can interact with a target molecule. For instance, the molecule responsive element can interact with cortisol or histamine. In some instances, the molecule responsive element binds to cortisol or histamine. Upon interaction of a target molecule with a molecule responsive element, said molecule responsive element promotes increased expression of a gene encoding, or increased production and/or secretion of, the response factor. Administration of the biosensing composition, or probiotic or pharmaceutical composition comprising the biosensing composition, disclosed herein can result in the production and/or secretion of a response factor, which can contribute to reduced levels or reduced activity of a target molecule. The production and/or secretion of a response factor disclosed herein can reduce the levels and/or activity of an intermediate molecule (i.e., a molecule that is not the target molecule). In some instances, of the methods disclosed herein, the response factor interacts with at least a second molecule that is not the target molecule within or on a tissue of a subject. The response factor may interact with a receptor and/or an intermediate molecule that interacts with the target molecule. In such instances, interaction of the response factor with a receptor and/or intermediate molecule may reduce interaction of said receptor and/or intermediate molecule with the target molecule.

In some embodiments of the methods disclosed herein, interaction of the molecule responsive element with the target molecule can result in an increased production and/or secretion of a response factor from the engineered yeast. The production and/or secretion of a response factor can be increased 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or greater than 10-fold, compared to a control yeast. A control yeast can include a yeast strain engineered with the molecule responsive element, but lacking the response factor disclosed herein, or a yeast strain lacking the molecule responsive element disclosed herein. A control yeast strain can also include any yeast strain lacking any component of the engineered yeast disclosed herein, such that the control yeast is incapable of producing and/or secreting the response factor in response to elevated target molecule levels. For instance, a control yeast may lack the activation domain upstream of the response factor that links its production to the molecule responsive element.

In some embodiments of the methods disclosed herein, interaction of the GC receptor with cortisol can result in an increased production and/or secretion of a response factor from the engineered yeast. The production and/or secretion of a response factor can be increased 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or greater than 10-fold, compared to a control yeast.

In some embodiments of the methods disclosed herein, wherein the engineered yeast comprises: a. a molecule responsive element comprising a polypeptide sequence comprising an amino acid sequence encoding a human glucocorticosteroid (GC) receptor, wherein said amino acid sequence is set forth as SEQ ID NO: 1, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 1, and/or a polynucleotide comprising a nucleotide sequence encoding the molecule responsive element, wherein said nucleotide sequence is set forth as SEQ ID NO: 2, or is a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 2; and b. a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, wherein the activation domain comprises a polynucleotide sequence set forth as: a. SEQ ID NO: 5, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polynucleotide sequence set forth as SEQ ID NO: 5; b. SEQ ID NO: 6, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polynucleotide sequence set forth as SEQ ID NO: 6; and/or c. SEQ ID NO: 11, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polynucleotide sequence set forth as SEQ ID NO: 11, interaction of the GC receptor with cortisol can result in an increased production and/or secretion of a response factor from the engineered yeast can be increased 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or greater than 10-fold, compared to a control yeast. In such instances, the engineered yeast can comprise Saccharomyces. In some embodiments, the engineered yeast comprises Saccharomyces cerevisiae. In some embodiments, the engineered yeast comprises Saccharomyces boulardii. Increased production and/or secretion of a response factor from the engineered yeast can be associated with decreased levels and/or activity of any molecule within or on the target tissue of the subject.

In some embodiments of the methods disclosed herein, wherein the engineered yeast comprises: a. a molecule responsive element comprising a GC receptor comprising an amino acid sequence set forth as SEQ ID NO: 1; and b. a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, wherein the activation domain comprises a polynucleotide sequence set forth as SEQ ID NO: 5, interaction of the GC receptor with cortisol can result in an increased production and/or secretion of a response factor from the engineered yeast, wherein said production and/or secretion is increased between 2-fold and 6.5-fold, compared to a control yeast. A control yeast strain can also include any yeast strain lacking any component of the engineered yeast, such that the control yeast is incapable of producing and/or secreting the response factor in response to elevated cortisol levels. For instance, a control yeast may lack the activation domain, or any sequence comprised within the activation domain, upstream of the response factor that links its production to the GC receptor.

In some embodiments of the methods disclosed herein, wherein the engineered yeast comprises: a. a molecule responsive element comprising a GC receptor comprising an amino acid sequence set forth as SEQ ID NO: 1; and b. a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, wherein the activation domain comprises a polynucleotide sequence set forth as SEQ ID NO: 6, interaction of the GC receptor with cortisol can result in an increased production and/or secretion of a response factor from the engineered yeast, wherein said production and/or secretion is increased between 2-fold and 6.5-fold, compared to a control yeast, wherein the engineered yeast comprises Saccharomyces. A control yeast strain can also include any yeast strain lacking any component of the engineered yeast, such that the control yeast is incapable of producing and/or secreting the response factor in response to elevated cortisol levels. For instance, a control yeast may lack the activation domain, or any sequence comprised within the activation domain, upstream of the response factor that links its production to the GC receptor.

In some embodiments of the methods disclosed herein, wherein the engineered yeast comprises: a. a molecule responsive element comprising a GC receptor comprising an amino acid sequence set forth as SEQ ID NO: 1; and b. a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, wherein the activation domain comprises a polynucleotide sequence set forth as SEQ ID NO: 11, interaction of the GC receptor with cortisol can result in an increased production and/or secretion of a response factor from the engineered yeast, wherein said production and/or secretion is increased between 2-fold and 6.5-fold, compared to a control yeast, wherein the engineered yeast comprises Saccharomyces. A control yeast strain can also include any yeast strain lacking any component of the engineered yeast, such that the control yeast is incapable of producing and/or secreting the response factor in response to elevated cortisol levels. For instance, a control yeast may lack the activation domain, or any sequence comprised within the activation domain, upstream of the response factor that links its production to the GC receptor.

In some embodiments of the methods disclosed herein, wherein the engineered yeast comprises: a. a polynucleotide sequence encoding the molecule responsive element comprising a nucleotide sequence set forth as SEQ ID NO: 2; c. a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, wherein the activation domain comprises a polynucleotide sequence set forth as SEQ ID NO: 5, interaction of the GC receptor with cortisol can result in an increased production and/or secretion of a response factor from the engineered yeast, wherein said production and/or secretion is increased between 2-fold and 6.5-fold, compared to a control yeast, wherein the engineered yeast comprises Saccharomyces. A control yeast strain can also include any yeast strain lacking any component of the engineered yeast, such that the control yeast is incapable of producing and/or secreting the response factor in response to elevated cortisol levels. For instance, a control yeast may lack the activation domain, or any sequence comprised within the activation domain, upstream of the response factor that links its production to the GC receptor.

In some embodiments of the methods disclosed herein, wherein the engineered yeast comprises: a. a polynucleotide sequence encoding the molecule responsive element comprising a nucleotide sequence set forth as SEQ ID NO: 2; c. a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, wherein the activation domain comprises a polynucleotide sequence set forth as SEQ ID NO: 6, interaction of the GC receptor with cortisol can result in an increased production and/or secretion of a response factor from the engineered yeast, wherein said production and/or secretion is increased between 2-fold and 6.5-fold, compared to a control yeast, wherein the engineered yeast comprises Saccharomyces. A control yeast strain can also include any yeast strain lacking any component of the engineered yeast, such that the control yeast is incapable of producing and/or secreting the response factor in response to elevated cortisol levels. For instance, a control yeast may lack the activation domain, or any sequence comprised within the activation domain, upstream of the response factor that links its production to the GC receptor.

In some embodiments of the methods disclosed herein, wherein the engineered yeast comprises: a. a polynucleotide sequence encoding the molecule responsive element comprising a nucleotide sequence set forth as SEQ ID NO: 2; c. a nucleic acid molecule comprising a polynucleotide encoding a response factor, wherein the nucleic acid molecule comprises an activation domain operably linked to the polynucleotide sequence encoding said response factor, and the activation domain comprises a promoter comprising a human glucocorticosteroid response element (HRE)-activated promoter, wherein the activation domain comprises a polynucleotide sequence set forth as SEQ ID NO: 11, interaction of the GC receptor with cortisol can result in an increased production and/or secretion of a response factor from the engineered yeast, wherein said production and/or secretion is increased between 2-fold and 6.5-fold, compared to a control yeast, wherein the engineered yeast comprises Saccharomyces. A control yeast strain can also include any yeast strain lacking any component of the engineered yeast, such that the control yeast is incapable of producing and/or secreting the response factor in response to elevated cortisol levels. For instance, a control yeast may lack the activation domain, or any sequence comprised within the activation domain, upstream of the response factor that links its production to the GC receptor.

In some embodiments of the methods disclosed herein, interaction of the GPCR with histamine can result in an increased production and/or secretion of a response factor from the engineered yeast. The production and/or secretion of a response factor can be increased 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or greater than 10-fold, compared to a control yeast.

In some embodiments of the methods disclosed herein, wherein the engineered yeast comprises: a. a molecule responsive element comprising binding domain that interacts with said target molecule, or a polynucleotide comprising a nucleotide sequence encoding a molecule responsive element; and b. a nucleic acid molecule comprising a polynucleotide encoding a response factor, whereby said interaction of said target molecule with said binding domain results in production and/or secretion of said response factor in said engineered yeast cells, wherein said molecule responsive element comprises a recombinant G-protein coupled receptor (GPCR) fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, and/or wherein the engineered yeast comprises a polynucleotide sequence encoding the molecule responsive element comprising a nucleotide sequence set forth as SEQ ID NO: 4, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 4, wherein said nucleic acid molecule comprising a polynucleotide sequence encoding a response factor comprises an activation domain operably linked to the polynucleotide sequence encoding said response factor, wherein said activation domain comprises a promoter comprising a Ste-12-activated promoter, wherein said plurality of engineered yeast cells further comprises: c. a modified GPA1 protein, or a polynucleotide comprising a nucleotide sequence encoding a modified GPA1 protein, wherein said modified GPA1 protein comprises an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8, interaction of the GPCR receptor with histamine can result in an increased production and/or secretion of a response factor from the engineered yeast can be increased at least 1.01-fold, 1.05-fold, 1.1-fold, 1.5-fold, 1.2-fold, or greater than 1.2-fold, compared to a control yeast.

In some embodiments of the methods disclosed herein, wherein the engineered yeast comprises: a. a molecule responsive element comprising a recombinant G-protein coupled receptor (GPCR) protein comprising an amino acid sequence set forth as SEQ ID NO: 3; b. a polynucleotide sequence encoding the molecule responsive element comprising a nucleotide sequence set forth as SEQ ID NO: 4; c. a nucleic acid molecule comprising a polynucleotide encoding a response factor operably linked to an activation domain comprising a promoter comprising a Ste-12-activated promoter; d. a modified GPA1 protein comprising an amino acid sequence set forth as SEQ ID NO: 8; and/or e. a polynucleotide comprising a nucleotide sequence encoding the modified GPA1 protein comprising a nucleotide sequence set forth as SEQ ID NO: 7, interaction of the GPCR receptor with histamine can result in an increased production and/or secretion of a response factor from the engineered yeast, wherein said production and/or secretion is increased between 1.01-fold and 1.2-fold, compared to a control yeast, wherein the engineered yeast comprises Saccharomyces. A control yeast strain can also include any yeast strain lacking any component of the engineered yeast, such that the control yeast is incapable of producing and/or secreting the response factor in response to elevated histamine levels. For instance, a control yeast may lack the GPCR receptor, or polynucleotide encoding the GPCR receptor, the modified GPA1 protein, and/or the activation domain, or any sequence comprised within the activation domain, upstream of the response factor that links its production to the GPCR receptor.

In some embodiments of the methods disclosed herein, the engineered yeast comprises: a. a polynucleotide sequence encoding the molecule responsive element comprising a nucleotide sequence set forth as SEQ ID NO: 4; b. a nucleic acid molecule comprising a polynucleotide encoding a response factor operably linked to an activation domain comprising a promoter comprising a Ste-12-activated promoter, wherein said activation domain comprises a nucleotide sequence set forth as SEQ ID NO: 12; and c. a polynucleotide comprising a nucleotide sequence encoding a modified GPA1 protein comprising a nucleotide sequence set forth as SEQ ID NO: 7, and interaction of the GPCR receptor with histamine results in an increased production and/or secretion of a response factor from the engineered yeast, wherein said production and/or secretion is increased between 1.01-fold and 1.2-fold, compared to a control yeast, wherein the engineered yeast comprises Saccharomyces.

In some embodiments of the methods disclosed herein, the engineered yeast comprises: a. a molecule responsive element comprising a recombinant G-protein coupled receptor (GPCR) protein comprising an amino acid sequence set forth as SEQ ID NO: 3; b. a nucleic acid molecule comprising a polynucleotide encoding a response factor operably linked to an activation domain comprising a promoter comprising a Ste-12-activated promoter, wherein said activation domain comprises a nucleotide sequence set forth as SEQ ID NO: 12; and c. a modified GPA1 protein comprising an amino acid sequence set forth as SEQ ID NO: 8, and interaction of the GPCR receptor with histamine results in an increased production and/or secretion of a response factor from the engineered yeast, wherein said production and/or secretion is increased between 1.01-fold and 1.2-fold, compared to a control yeast, wherein the engineered yeast comprises Saccharomyces.

In some embodiments of the methods disclosed herein, the engineered yeast comprises: a. a molecule responsive element comprising a recombinant G-protein coupled receptor (GPCR) protein comprising an amino acid sequence set forth as SEQ ID NO: 3; b. a polynucleotide sequence encoding the molecule responsive element comprising a nucleotide sequence set forth as SEQ ID NO: 4; c. a nucleic acid molecule comprising a polynucleotide encoding a response factor operably linked to an activation domain comprising a promoter comprising a Ste-12-activated promoter, wherein said activation domain comprises a nucleotide sequence set forth as SEQ ID NO: 12; d. a modified GPA1 protein comprising an amino acid sequence set forth as SEQ ID NO: 8; and e. a polynucleotide comprising a nucleotide sequence encoding the modified GPA1 protein comprising a nucleotide sequence set forth as SEQ ID NO: 7, and interaction of the GPCR receptor with histamine can result in an increased production and/or secretion of a response factor from the engineered yeast, wherein said production and/or secretion is increased between 1.01-fold and 1.2-fold, compared to a control yeast, wherein the engineered yeast comprises Saccharomyces. A control yeast strain can also include any yeast strain lacking any component of the engineered yeast, such that the control yeast is incapable of producing and/or secreting the response factor in response to elevated histamine levels. For instance, a control yeast may lack the GPCR receptor, or polynucleotide encoding the GPCR receptor, the modified GPA1 protein, and/or the activation domain, or any sequence comprised within the activation domain, upstream of the response factor that links its production to the GPCR receptor.

A control yeast strain can also include any yeast strain lacking any component of the engineered yeast, such that the control yeast is incapable of producing and/or secreting the response factor in response to elevated histamine levels. For instance, a control yeast may lack the GPCR receptor, or polynucleotide encoding the GPCR receptor, the modified GPA1 protein, and/or the activation domain, or any sequence comprised within the activation domain, upstream of the response factor that links its production to the GPCR receptor.

In the methods disclosed herein, the target molecule can comprise a ligand, a protein, metabolite, a hormone, a nutrient, or small molecule. Disclosed herein are methods, wherein the target molecule comprises cortisol or histamine. The target molecule can be present on the skin or in the esophagus, nasal tissue, mouth, vaginal tissue, lungs, stomach, small intestines, large intestines, digestive tract, or gastrointestinal tract of said subject. The target molecule can be present at elevated levels on the skin or in the esophagus, nasal tissue, mouth, vaginal tissue, lungs, stomach, small intestines, large intestines, digestive tract, or gastrointestinal tract of said subject.

Following administration of an effective amount of a biosensing composition to a subject, the levels of said target molecule will be reduced in said subject. The levels of the target molecule can be reduced on the skin or in the esophagus, nasal tissue, mouth, vaginal tissue, lungs, stomach, small intestines, large intestines, digestive tract, or gastrointestinal tract of said subject. The target molecule that is reduced according to the disclosed methods is produced by said subject. The target molecule can be produced in the tissue that it is bound, detecting and/or reduced, or it can be produced in a different tissue.

In any of the methods of treating a disease, or disorder, or symptom associated with a disease or disorder disclosed herein, production and/or secretion of a response factor may function to treat and/or reduce a sign or symptom associated with a disease or disorder or dysbiosis. A response factor may act on any molecule within or on a tissue that directly and/or indirectly promotes a sign or symptom associated with a disease or disorder or dysbiosis.

In any of the methods of maintaining a healthy biological function within a tissue of a subject disclosed herein, production and/or secretion of a response factor may function to treat and/or reduce a sign or symptom associated with a disease or disorder or dysbiosis. A response factor may act on any molecule within or on a tissue that directly and/or indirectly promotes a sign or symptom associated with a disease or disorder or dysbiosis.

In some instances, a response factor promotes the reduction in levels or functionality of a molecule that promotes a sign or symptom associated with a disease or disorder or dysbiosis. In such instances, the molecule may be the target molecule. In some instances, the molecule may be an intermediate molecule that is not the target molecule.

The biosensing composition, probiotic, or pharmaceutical composition can be administered at least once a day, at least one a week, at least twice a week, at least once a month, at least twice a month, at least once a year, or at least twice a year.

A pharmaceutical composition is a composition that is employed to prevent, reduce in intensity, cure or otherwise treat a disease or disorder that comprises an active ingredient and a pharmaceutically acceptable carrier. A pharmaceutically acceptable carrier refers to one or more compatible solid or liquid filler, diluents or encapsulating substances which are suitable for administration to a human or other vertebrate animal. In some embodiments, the pharmaceutical composition comprises a pharmaceutically acceptable carrier that is non-naturally occurring.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. Non-limiting examples include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. Administered intravenously, particular carriers are physiological saline or phosphate buffered saline (PBS). Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc. These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Pharmaceutical compositions comprising the biosensing composition can be formulated in order to maximize the effectiveness of the biosensing composition and can be tailored to specific sensing and response activity of each individual biosensing composition. For example, the pharmaceutical compositions disclosed herein can be formulated as controlled release formulations for sustained or timed release of the active components to enhance efficacy and reduce dosing frequency. Microencapsulation of the pharmaceutical composition can be used to protect the probiotic yeast from harsh gastrointestinal conditions, ensuring viability and functionality upon reaching target sites. Combination Therapies and/or supplements of the pharmaceutical composition can be developed that include compositions combining the biosensing probiotics with other therapeutic agents or non-therapeutic agents (consumer side, fracture-function claims) for synergistic effects (e.g., anti-inflammatory drug or herbal extract, other probiotic strain, etc). Advanced delivery systems of the pharmaceutical composition can be used, such as nanoparticles, liposomes, or hydrogels that could enhance delivery efficiency and targeting pharmaceutical compositions comprising the biosensing compositions can be administered to a subject via any route, such as orally, buccally, parenterally, topically, by inhalation or insufflation (i.e., through the mouth or through the nose), or rectally. Systemic administration of the biosensing composition disclosed herein can be achieved by intravenous administration of the pharmaceutical composition comprising the biosensing composition. Administering can be performed, for example, once, a plurality of times, and/or over one or more extended periods. Oral administration is one particular method of administration. The biosensing compositions can be administered in the form of a capsule, gel, softgel, paste, tablet, powder, gummy, spray, aerosol, lozenge, effervescent tablet, orodispersible film, topical cream, ointment, suppository, emulsion, transdermal patch, or liquid. The biosensing compositions and/or probiotic comprising the same can be formulated into a nutritional whole food, drink, sports drink, coffee, tea, water, mineral water, soda, carbonated beverage, oral hydration drink, mineral water, soup, replacement food, nutritional bar, nutritional consumable, gummy, tablet, confectionery, fermented or unfermented milk-based product, yogurt product, milk-based powder, enteral nutritional product, fermented or unfermented cereal-based product, chocolate, milk, yogurt, cheese, ice cream, baby and infant formula, cereal, animal feed, protein shake, protein powder, functional water, juice blend, smoothie, ready-to-drink (RTD) beverage, non-dairy milk, nut or seed butter, energy drink, functional shot, baked good, granola bar, snack bar, alcoholic beverage, frozen food, frozen meal, condiment, pasta, noodles, pet food, or pet treat.

An effective amount of a pharmaceutical composition of the invention is any amount that is effective to achieve its purpose (e.g., prevention of or recovery from, including partial recovery, or prevention or slowing of disorder or disease caused by a specific sequence). The effective amount, usually expressed in mg/kg can be determined by routine methods during pre-clinical and clinical trials by those of skill in the art.

### V. Methods of Manufacturing Biosensing Compositions

The present disclosure provides methods of manufacturing a biosensing composition comprising an engineered microorganism, such as an engineered yeast cell, or a plurality of engineered microorganisms, comprising a molecule responsive element comprising a polypeptide, or a polynucleotide comprising a nucleotide sequence encoding the molecule responsive element, and nucleic acid molecule encoding a response factor. The methods can involve generating an engineered yeast cell comprising any molecule responsive element, or polynucleotide encoding a molecule responsive element, disclosed herein. In the engineered yeast cell comprising a molecule responsive element comprising a polypeptide, and nucleic acid molecule encoding a response factor, interaction of a target molecule with the binding domain of the molecule responsive element can result in the production of the response factor. As such the engineered yeast can further comprise a signaling pathway that functions to transmit an intracellular signal from the molecule responsive element to activate expression of a gene encoding the response factor.

Disclosed herein are methods of manufacturing a biosensing composition comprising the steps of: a) introducing into a yeast cell a nucleic acid molecule comprising a polynucleotide comprising a nucleotide sequence encoding a molecule responsive element comprising a binding domain that interacts with a target molecule, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said molecule responsive element and said polynucleotide encoding said response factor.

In some instances, interaction of the target molecule with the molecule response element results in expression of the polynucleotide encoding the response factor. In some instances, gene expression is activated by a signaling pathway that is activated upon interaction of the target molecule with the molecule responsive element. In some instances, interaction of the target molecule with the binding domain of the molecule response element triggers a signaling pathway that leads to activation of the polynucleotide encoding the response factor. As such, the methods of manufacturing disclosed herein can involve yeast cells comprising signaling pathways. Any signaling pathway that enables a molecule responsive element to activate downstream gene expression of a response factor can be included in the methods. For instance, the engineered yeast can comprise a native yeast signaling pathway. In some instances, the engineered yeast comprises a recombinant signaling pathway. As described elsewhere herein, a person having ordinary skill in the art would understand a signaling pathway to include multiple components (e.g., proteins) that play a role in transmitting a signal from outside of the cell (e.g., the presence and detection of a target molecule by a molecule responsive element) to activate gene expression (e.g., of a response factor) within the cell. Any component of the signaling pathway of the engineered yeast can be recombinant. In some instances, gene expression is activated directly by the molecule response element. In such instances, the molecule response element can be bound, or complexed, with the target molecule to activate gene expression.

The biosensing compositions disclosed herein comprise modified microorganisms. The microorganism can be any microorganism disclosed herein. In a particular embodiment, the microorganism can comprise yeast. Any species, strain, and/or substrain of yeast is envisaged as being comprised within the biosensing composition disclosed herein. In a particular embodiment, the engineered yeast is Saccharomyces cerevisiae. In another particular embodiment, the engineered yeast is Saccharomyces boulardii. Also disclosed are compositions comprising a plurality of engineered yeast cells. The plurality of engineered yeast cells can comprise any yeast cell described herein, such as Saccharomyces cerevisiae and/or Saccharomyces boulardii.

The methods disclosed herein can comprise manufacturing of any biosensing composition disclosed herein. The methods can involve generating an engineered yeast cell comprising a molecule responsive element, or a polynucleotide comprising a nucleotide sequence encoding the molecule responsive element, and/or a nucleic acid molecule comprising a polynucleotide comprising a nucleotide sequence encoding a response factor. A yeast cell can be engineered to comprise a molecule responsive element, or a polynucleotide comprising a nucleotide sequence encoding the molecule responsive element, and/or a nucleic acid molecule comprising a polynucleotide comprising a nucleotide sequence encoding a response factor.

Also disclosed herein are yeast populations comprising a plurality of any engineered microorganism generated in the present disclosure. In some embodiments, the yeast population comprises a plurality of any engineered Saccharomyces cells.

In some instances, the methods disclosed herein provide a method of manufacturing a biosensing composition comprising a plurality of Saccharomyces cerevisiae and/or Saccharomyces boulardii that are useful for binding, detecting, and/or reducing the levels of a target molecule in a subject. In some instances, the methods disclosed herein provide a method of manufacturing a biosensing composition comprising a plurality of Saccharomyces cerevisiae and/or Saccharomyces boulardii that are useful for treating a disease and/or disorder or reducing at least one sign or symptom of a disease and/or disorder in a subject. In some instances, the methods disclosed herein provide a method of manufacturing a biosensing composition comprising a plurality of Saccharomyces cerevisiae and/or Saccharomyces boulardii that are useful for maintaining a healthy tissue in a subject.

The methods of manufacturing a biosensing composition disclosed herein can comprise the steps of: a) introducing into a yeast cell a nucleic acid molecule comprising a polynucleotide comprising a nucleotide sequence encoding a molecule responsive element comprising a binding domain that interacts with a target molecule, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said molecule responsive element and said polynucleotide encoding said response factor. In some instances, the method further comprises the step of selecting for yeast cells comprising said polynucleotide encoding said molecule responsive element and/or said polynucleotide encoding said response factor. Methods of introducing nucleic acids into yeast cells will be known to those having ordinary skill in the art. Methods for culturing and/or selecting specific microorganisms, such as those within a culture, that comprise certain nucleic acids are known in the art. Any method can be used. For instance, nucleic acids can comprise an antibiotic selection marker that bestows resistance to the microorganism comprising the nucleic acid molecule. Culturing the microorganism in the presence of a selection marker, such as an antibiotic, would be expected to kill or remove all microorganisms that do not comprise any of the nucleic acid molecules that do not comprise resistance to the selection marker. Following a selection step, at least 50%, at least 55%, at least 60%, at least 65%, at least 70%, at least 75%, at least 80%, at least 85%, at least 90%, at least 95%, or at least 99% of the engineered yeast cells within said plurality comprises said polynucleotide encoding said molecule responsive element and said polynucleotide encoding said response factor.

Disclosed herein are methods of manufacturing a biosensing composition comprising the step of: introducing into a yeast cell a nucleic acid molecule comprising a polynucleotide comprising a nucleotide sequence encoding a molecule responsive element comprising a binding domain that interacts with a target molecule. In some instances, the molecule responsive element comprises a polypeptide. In some instances, the molecule responsive element comprises a nucleic acid molecule. The molecule responsive element can comprise a protein, such as a fusion protein, a transmembrane protein, a receptor, and/or a functional fragment or variant thereof.

In some instances, the methods of manufacturing a biosensing composition disclosed herein comprises introducing into a yeast cell a polynucleotide comprising a nucleotide sequence encoding a molecule responsive element comprising a receptor protein, wherein said receptor protein comprises a human glucocorticosteroid (GC) receptor, or a functional fragment or variant thereof having at least 80%, 81%, 82%, 83%, 84%, 85%, 86%, 87%, 88%, 89%, 90%, 91%, 92%, 93%, 94%, 95%, 96%, 97%, 98%, 99%, or 100% to a human glucocorticosteroid (GC) receptor. The polynucleotide can comprise a nucleotide sequence encoding a molecule responsive element comprising a receptor protein comprising a human GC receptor, wherein said GC receptor comprises an amino acid sequence set forth as SEQ ID NO: 1, or a functional fragment or variant thereof having at least 80%, at least 85%, at least 90%, at least 95%, or at least 99% sequence identity to the amino acid sequence set forth as SEQ ID NO: 1.

In some instances of the methods of manufacturing a biosensing composition disclosed herein, the polynucleotide can comprise a nucleotide sequence encoding a molecule responsive element a human GC receptor, wherein said GC receptor comprises an amino acid sequence set forth as SEQ ID NO: 1. In such instances, the polynucleotide can comprise a nucleotide sequence set forth as SEQ ID NO: 2, or a variant thereof having at least 80%, at least 85%, at least 90%, at least 95%, or at least 99% sequence identity to the nucleotide sequence set forth as SEQ ID NO: 2.

In some instances of the methods of manufacturing a biosensing composition disclosed herein, the polynucleotide comprising a nucleotide sequence encoding a molecule responsive element comprising a GC receptor can comprise a nucleotide sequence set forth as SEQ ID NO: 2.

In some instances, the methods of manufacturing a biosensing composition disclosed herein comprises introducing into a yeast cell a polynucleotide comprising a nucleotide sequence encoding a molecule responsive element comprising a receptor protein, wherein said receptor protein comprises a G-protein coupled receptor (GPCR) protein, or a functional fragment or variant thereof having at least 80%, 81%, 82%, 83%, 84%, 85%, 86%, 87%, 88%, 89%, 90%, 91%, 92%, 93%, 94%, 95%, 96%, 97%, 98%, 99%, or 100% to a G-protein coupled receptor (GPCR) protein. In some instances, the methods of manufacturing a biosensing composition disclosed herein comprises introducing into a yeast cell a polynucleotide comprising a nucleotide sequence encoding a molecule responsive element comprising a receptor protein, wherein said receptor protein comprises a human G-protein coupled receptor (GPCR) protein, or a functional fragment or variant thereof having at least 80%, 81%, 82%, 83%, 84%, 85%, 86%, 87%, 88%, 89%, 90%, 91%, 92%, 93%, 94%, 95%, 96%, 97%, 98%, 99%, or 100% to a human G-protein coupled receptor (GPCR) protein. In some instances, the methods of manufacturing a biosensing composition disclosed herein comprises introducing into a yeast cell a polynucleotide comprising a nucleotide sequence encoding a molecule responsive element comprising a receptor protein, wherein said receptor protein comprises a yeast G-protein coupled receptor (GPCR) protein, or a functional fragment or variant thereof having at least 80%, 81%, 82%, 83%, 84%, 85%, 86%, 87%, 88%, 89%, 90%, 91%, 92%, 93%, 94%, 95%, 96%, 97%, 98%, 99%, or 100% to a yeast G-protein coupled receptor (GPCR) protein. The polynucleotide can comprise a nucleotide sequence encoding a molecule responsive element comprising a receptor protein comprising a human G-protein coupled receptor (GPCR) protein, wherein said a human G-protein coupled receptor (GPCR) protein comprises an amino acid sequence set forth as SEQ ID NO: 3, or a functional fragment or variant thereof having at least 80%, at least 85%, at least 90%, at least 95%, or at least 99% sequence identity to the amino acid sequence set forth as SEQ ID NO: 3.

In some instances of the methods of manufacturing a biosensing composition disclosed herein, the polynucleotide can comprise a nucleotide sequence encoding a molecule responsive element a human G-protein coupled receptor (GPCR) protein, wherein said a human G-protein coupled receptor (GPCR) protein comprises an amino acid sequence set forth as SEQ ID NO: 3. In such instances, the polynucleotide can comprise a nucleotide sequence set forth as SEQ ID NO: 4, or a variant thereof having at least 80%, at least 85%, at least 90%, at least 95%, or at least 99% sequence identity to the nucleotide sequence set forth as SEQ ID NO: 4.

In some instances of the methods of manufacturing a biosensing composition disclosed herein, the polynucleotide comprising a nucleotide sequence encoding a molecule responsive element comprising a human G-protein coupled receptor (GPCR) protein can comprise a nucleotide sequence set forth as SEQ ID NO: 4.

Disclosed herein are methods of manufacturing a biosensing composition, the method comprising the steps of: a) introducing into a yeast cell a nucleic acid molecule comprising a polynucleotide comprising a nucleotide sequence encoding a GC receptor comprising an amino acid sequence set forth as SEQ ID NO: 1, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said GC receptor and said polynucleotide encoding said response factor.

Disclosed herein are methods of manufacturing a biosensing composition, the method comprising the steps of: a) introducing into a yeast cell a nucleic acid molecule comprising a polynucleotide comprising a nucleotide sequence encoding a GC receptor, wherein said polynucleotide comprises a nucleotide sequence set forth as SEQ ID NO: 2, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said GC receptor and said polynucleotide encoding said response factor.

The methods of manufacturing a biosensing composition disclosed herein can further comprise the step of: introducing into said yeast cell a polynucleotide comprising a nucleotide sequence encoding a polypeptide encoding a modified yeast GPA1 protein. In such instances, the polypeptide encoding a modified yeast GPA1 protein: a. comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10; b. comprises an amino acid sequence corresponding with residues 1-467 of SEQ ID NO: 10; c. comprises an amino acid sequence comprising one or more than one amino acid substitution compared to SEQ ID NO: 10, wherein said one or more than one amino acid substitution are selected from the following: K468E, 1469Y, G470N, 1471L, and 1472V; d. comprises an amino acid sequence comprising the following amino acid substitutions: K468E, I469Y, G470N, I471L, and I472V, compared to SEQ ID NO: 10; and/or e. comprises an amino acid sequence comprising an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8; and/or wherein said polynucleotide comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein comprising an amino acid sequence set forth as SEQ ID NO: 3, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein comprising an amino acid sequence set forth as SEQ ID NO: 8.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein, wherein said polynucleotide comprises a nucleotide sequence set forth as SEQ ID NO: 4, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein comprising an amino acid sequence set forth as SEQ ID NO: 8.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein comprising an amino acid sequence set forth as SEQ ID NO: 3, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein, wherein said polynucleotide comprises a nucleotide sequence set forth as SEQ ID NO: 7.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein, wherein said polynucleotide comprises a nucleotide sequence set forth as SEQ ID NO: 4, b) introducing into said yeast cell a nucleic acid molecule comprising a polynucleotide sequence encoding a response factor, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein, wherein said polynucleotide comprises a nucleotide sequence set forth as SEQ ID NO: 7.

The introduction of the nucleic acid molecule comprising the molecule responsive element and the polynucleotide encoding the response factor can occur simultaneously. Alternatively, introduction of the nucleic acid molecule comprising the molecule responsive element and the polynucleotide encoding the response factor can occur sequentially. When introduction of multiple polynucleotides into the engineered yeast occurs sequentially, the introduction can occur in any order. For instance, introduction of the polynucleotide encoding the molecule responsive element can occur prior to or after introduction of the polynucleotide encoding a response factor. The polynucleotides can be codon-optimized for the specific microorganism into which they are introduced. For example, the polynucleotides can be codon-optimized for yeast cells. Additionally, the nucleic acid molecule comprising the molecule responsive element and the polynucleotide encoding the response factor can be comprised within a single nucleic acid molecule, such as a plasmid or a linear nucleic acid molecule, such as double-stranded or single-stranded nucleic acid. Alternatively, the nucleic acid molecule comprising the molecule responsive element and the polynucleotide encoding the response factor can be comprised within different nucleic acid molecules. The polynucleotides can be integrated into the chromosome of the microorganism. Alternatively, the polynucleotides can be maintained outside of the chromosome. In some instances, one polynucleotide is integrated into the chromosome and the other is maintained outside of the chromosome.

The methods disclosed herein can be used to generate an engineered microorganism. Any species of microorganism can be engineered. The methods can be used to generate an engineered yeast cell, or a plurality of engineered yeast cells. The yeast can comprise Saccharomyces, such as Saccharomyces cerevisiae and/or Saccharomyces boulardii. The Saccharomyces cerevisiae and/or Saccharomyces boulardii can comprise at least one genomic modification. In some instances, the Saccharomyces cerevisiae and/or Saccharomyces boulardii comprises mutation and/or deletion in at least one of the following genes: STE2, FAR1, SST2, and MTA-A.

A polynucleotide encoding any molecule responsive element disclosed herein can be introduced into the microorganism. The molecule responsive element can comprise a ligand, a protein, metabolite, a hormone, a nutrient, or small molecule, such as cortisol or histamine. For instance, the molecule responsive element can interact with cortisol or histamine.

The molecule responsive element can comprise a polypeptide or a polynucleotide.

In a particular embodiment, a polynucleotide encoding a molecule responsive element comprising a human glucocorticosteroid (GC) receptor is introduced into Saccharomyces cerevisiae and/or Saccharomyces boulardii.

In another particular embodiment, a polynucleotide encoding a molecule responsive element comprising a recombinant G-protein coupled receptor (GPCR) fusion protein is introduced into Saccharomyces cerevisiae and/or Saccharomyces boulardii. In some instances, the GPCR fusion protein comprises a histamine binding ectodomain. The GPCR fusion protein can comprise an intracellular signaling domain comprises a yeast protein intracellular signaling domain, or a fragment or variant thereof having at least 80%, at least 85%, at least 90%, at least 95%, or at least 99% sequence identity to a yeast protein intracellular signaling domain.

A polynucleotide encoding a molecule responsive element that interacts with or binds to cortisol can be introduced into Saccharomyces cerevisiae and/or Saccharomyces boulardii simultaneously or sequentially to a polynucleotide encoding a response factor. A polynucleotide encoding a molecule responsive element that interacts with or binds to histamine can be introduced into Saccharomyces cerevisiae and/or Saccharomyces boulardii simultaneously or sequentially to a polynucleotide encoding a response factor.

The present disclosure also provides a nucleic acid encoding the molecule responsive element and the response factor, as well as a host cell comprising said nucleic acid. The host cell can comprise a microorganism. In some instances, the microorganism is a bacteria cell. In some instances, the microorganism is a probiotic organism. Any probiotic organism can be engineered with the molecule responsive elements and response factors, or polynucleotides encoding the molecule responsive elements and response factors disclosed herein. In some instances, the host cell comprises a yeast cell. In a particular embodiment, the host cell comprises Saccharomyces. In another embodiment, the host cell comprises Saccharomyces cerevisiae. In another embodiment, the host cell comprises Saccharomyces boulardii.

Also disclosed herein are polynucleotides comprising a nucleotide sequence encoding a human glucocorticosteroid (GC) receptor, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a human glucocorticosteroid (GC) receptor. Polynucleotides encoding a human GC receptor or variant thereof can be introduced into a yeast cell using any method known in the art. In some instances, the polynucleotide comprises a nucleotide sequence set forth as SEQ ID NO: 2, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 2.

Also disclosed herein are polynucleotides comprising a nucleotide sequence encoding a human G-protein coupled receptor (GPCR) protein, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a human G-protein coupled receptor (GPCR) protein. Polynucleotides encoding a human G-protein coupled receptor (GPCR) protein or variant thereof can be introduced into a yeast cell using any method known in the art. In some instances, the polynucleotide comprises a nucleotide sequence set forth as SEQ ID NO: 4, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 4.

Also disclosed herein are polynucleotides comprising a nucleotide sequence encoding a modified yeast GPA1 protein. The modified yeast GPA1 protein can have at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to a native (i.e., wild-type) yeast GPA1 protein. Polynucleotides encoding a modified yeast GPA1 can be introduced into a yeast cell using any method known in the art. In some instances, the polynucleotide comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7. In some instances, the polynucleotide comprises a nucleotide sequence encoding a modified GPA1 protein comprising an amino acid having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 10. In some instances, the polynucleotide comprises a nucleotide sequence encoding a modified GPA1 protein comprises an amino acid sequence corresponding with residues 1-467 of SEQ ID NO: 10. In some instances, the polynucleotide comprises a nucleotide sequence encoding a modified GPA1 protein comprising an amino acid sequence comprising one or more than one amino acid substitution compared to SEQ ID NO: 10, wherein said one or more than one amino acid substitution is selected from the following: K468E, I469Y, G470N, I471L, and I472V. In some instances, the polynucleotide comprises a nucleotide sequence encoding a modified GPA1 protein comprises an amino acid sequence comprising the following amino acid substitutions: K468E, I469Y, G470N, I471L, and I472V, compared to SEQ ID NO: 10. In some instances, the polynucleotide comprises a nucleotide sequence encoding a modified GPA1 protein comprises an amino acid sequence comprising an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8.

The polynucleotides disclosed herein can be codon-optimized for expression in yeast. In some instances, the polynucleotide comprising a nucleotide sequence encoding a human GC receptor is codon-optimized for expression in yeast. In some instances, the polynucleotide comprising a nucleotide sequence encoding a G-protein coupled receptor (GPCR) protein is codon-optimized for expression in yeast.

The nucleic acids can be used to produce a presently disclosed host cell, such as a yeast cell. In particular, the nucleic acid comprising the molecule responsive element can be introduced into the host cell such that it is operably linked to an inducible or constitutively active promoter element. In certain embodiments, the host cell is a yeast cell, and a nucleic acid encoding a molecule responsive element is introduced into the yeast cell either as a construct or a plasmid in which it is operably linked to a promoter active in the yeast cell or such that it is inserted into the yeast cell genome at a location where it is operably linked to a suitable promoter. Non-limiting examples of suitable yeast promoters include, but are not limited to, constitutive promoters pGAP, pTef1, pPgk1, pCyc1, pAdh1, pKex1, pTdh3, pTpi1, pPyk1, and pHxt7 and inducible promoters pGal1, pCup1, pMet15, and pFus1. In some instances, wherein the host cell is a yeast cell, the molecule responsive element is operably linked to a GAP promoter. In some instances, wherein the host cell is a yeast cell, the molecule responsive element is operably linked to a promoter comprising a polynucleotide sequence set forth as SEQ ID NO: 12, or a functional variant having at least 80%, at least 85%, at least 90%, at least 95%, or greater than 95% sequence homology to SEQ ID NO: 12. A functional variant when used to refer to a promoter sequence operably linked to a polynucleotide sequence encoding a molecule responsive element is understood to be a sequence that retains the ability to bind to RNA polymerase and/or any required transcription factors necessary to initiate and drive gene expression (i.e., transcription) of the molecule responsive element. In a specific embodiment, wherein the host cell is a yeast cell comprising a polynucleotide comprising a nucleotide sequence encoding a molecule responsive element comprising a human GPCR fusion protein comprising a histamine binding domain, wherein said molecule responsive element comprises an amino acid sequence set forth as SEQ ID NO: 3, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 3, the polynucleotide sequence encoding the molecule responsive element is operably linked to a promoter comprising a polynucleotide sequence set forth as SEQ ID NO: 12, or a functional variant having at least 80%, at least 85%, at least 90%, at least 95%, or greater than 95% sequence homology to SEQ ID NO: 12. In another specific embodiment, wherein the host cell is a yeast cell comprising a polynucleotide comprising a nucleotide sequence encoding a molecule responsive element comprising a recombinant GPCR fusion protein comprising a histamine binding domain, wherein said molecule responsive element comprises an amino acid sequence set forth as SEQ ID NO: 3, the polynucleotide sequence encoding the molecule responsive element is operably linked to a promoter comprising a polynucleotide sequence set forth as SEQ ID NO: 12.

The use of the term “polynucleotide” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. The polynucleotides disclosed herein also encompass all forms of sequences including, but not limited to, single-stranded forms, double-stranded forms, stem-and-loop structures, and the like.

In some instances of the methods of manufacturing a biosensing composition, the polynucleotide comprising a nucleotide sequence encoding the response factor can comprise an activation domain operably linked to the polynucleotide sequence encoding the response factor. The activation domain can be operably linked to the polynucleotide encoding the response factor in order to specifically activate gene expression of the response factor in response interaction of a target molecule with the molecule responsive element. As such, a specific activation domain that only is activated by a molecule responsive element, or a signal transduction pathway that functions with the molecule responsive element, can be operably linked to a polynucleotide encoding a response factor. For instance, the activation domain can comprise a promoter, such as a Ste-12-activated or a human glucocorticosteroid response element (HRE)-activated promoter. In such instances, the response factor is produced upon binding of STE12 or GC receptor to said promoter. The activation domain can comprise any promoter sequence described herein.

Disclosed herein are methods of manufacturing a biosensing composition, the method comprising the steps of: a) introducing into a yeast cell a nucleic acid molecule comprising a polynucleotide comprising a nucleotide sequence encoding a GC receptor comprising an amino acid sequence set forth as SEQ ID NO: 1, b) introducing into said yeast cell a nucleic acid molecule comprising a polynucleotide sequence encoding a response factor, wherein said polynucleotide sequence encoding said response factor is operably fused to an activation domain comprising a nucleotide sequence set forth as SEQ ID NO: 5, 6, and/or 11, and c) culturing said engineered yeast cell under conditions sufficient to allow production of a yeast cell culture comprising a plurality of engineered yeast cells comprising said polynucleotide encoding said GC receptor and said polynucleotide encoding said response factor, wherein said yeast cell comprises Saccharomyces cerevisiae and/or Saccharomyces boulardii.

Disclosed herein are methods of manufacturing a biosensing composition, the method comprising the steps of: a) introducing into a yeast cell a nucleic acid molecule comprising a polynucleotide comprising a nucleotide sequence encoding a GC receptor, wherein said polynucleotide comprises a nucleotide sequence set forth as SEQ ID NO: 2, b) introducing into said yeast cell a nucleic acid molecule comprising a polynucleotide sequence encoding a response factor, wherein said polynucleotide sequence encoding said response factor is operably fused to an activation domain comprising a nucleotide sequence set forth as SEQ ID NO: 5, 6, and/or 11, and c) culturing said engineered yeast cell under conditions sufficient to allow production of a yeast cell culture comprising a plurality of engineered yeast cells comprising said polynucleotide encoding said GC receptor and said polynucleotide encoding said response factor, wherein said yeast cell comprises Saccharomyces cerevisiae and/or Saccharomyces boulardii.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein comprising an amino acid sequence set forth as SEQ ID NO: 3, b) introducing into said yeast cell a nucleic acid molecule comprising a polynucleotide sequence encoding a response factor, wherein said polynucleotide sequence encoding said response factor is operably fused to an activation domain comprising a Ste12 activated promoter, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein comprising an amino acid sequence set forth as SEQ ID NO: 8, wherein said yeast cell comprises Saccharomyces cerevisiae and/or Saccharomyces boulardii, wherein said method further comprises the step of introducing at least one genomic modification in the yeast in the following genes: STE2, FAR1, SST2, and MTA-A.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein, wherein said polynucleotide comprises a nucleotide sequence set forth as SEQ ID NO: 4, b) introducing into said yeast cell a nucleic acid molecule comprising a polynucleotide sequence encoding a response factor, wherein said polynucleotide sequence encoding said response factor is operably fused to an activation domain comprising a Ste12 activated promoter, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein comprising an amino acid sequence set forth as SEQ ID NO: 8, wherein said yeast cell comprises Saccharomyces cerevisiae and/or Saccharomyces boulardii, wherein said method further comprises the step of introducing at least one genomic modification in the yeast in the following genes: STE2, FAR1, SST2, and MTA-A.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein comprising an amino acid sequence set forth as SEQ ID NO: 3, b) introducing into said yeast cell a nucleic acid molecule comprising a polynucleotide sequence encoding a response factor, wherein said polynucleotide sequence encoding said response factor is operably fused to an activation domain comprising a Ste12 activated promoter, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein, wherein said polynucleotide comprises a nucleotide sequence set forth as SEQ ID NO: 7, wherein said yeast cell comprises Saccharomyces cerevisiae and/or Saccharomyces boulardii, wherein said method further comprises the step of introducing at least one genomic modification in the yeast in the following genes: STE2, FAR1, SST2, and MTA-A.

Disclosed herein are methods of manufacturing a biosensing composition, the method comprising the steps of: a) introducing into a yeast cell a nucleic acid molecule comprising a polynucleotide comprising a nucleotide sequence encoding a human G-protein coupled receptor (GPCR) protein, wherein said polynucleotide comprises a nucleotide sequence set forth as SEQ ID NO: 4, b) introducing into said yeast cell a nucleic acid molecule comprising a polynucleotide sequence encoding a response factor, wherein said polynucleotide sequence encoding said response factor is operably fused to an activation domain comprising a Ste12 activated promoter, and c) culturing said engineered yeast cell under conditions sufficient to allow production of a yeast cell culture comprising a plurality of engineered yeast cells comprising said polynucleotide encoding said human G-protein coupled receptor (GPCR) protein and said polynucleotide encoding said response factor, wherein said method further comprises the step of: introducing into said yeast cell a polynucleotide comprising a nucleotide sequence encoding a polypeptide encoding a modified yeast GPA1 protein, wherein said polynucleotide comprises a nucleotide sequence set forth as SEQ ID NO: 7, wherein said yeast cell comprises Saccharomyces cerevisiae and/or Saccharomyces boulardii, wherein said method further comprises the step of introducing at least one genomic modification in the yeast in the following genes: STE2, FAR1, SST2, and MTA-A.

The methods of manufacturing a biosensing composition disclosed herein can comprise a plurality of microorganisms suitable for binding, detecting, and/or reducing the levels of a target molecule in a subject. Said plurality can comprise any microorganism suitable for administration to a subject (e.g., non-pathogenic microorganism). In some instances, the method comprises a plurality comprising a probiotic microorganism. Probiotic microorganisms are known in the art. Any probiotic microorganism is envisaged within the scope of the presently disclosed methods. In some instances, the plurality comprises a single species of microorganism. In some instances, the plurality comprises more than one species of microorganism. In some instances, wherein the plurality comprises more than one species, a first species comprises Saccharomyces cerevisiae and a second species comprises Saccharomyces boulardii. The plurality comprising more than one species of microorganism can comprise a first species comprising Saccharomyces cerevisiae or Saccharomyces boulardii and at least a second species.

The methods of manufacturing a biosensing composition disclosed herein generate engineered yeast cells that can produce an effective amount of said response factor in response to a threshold concentration of said target molecule. When said target molecule is cortisol, said threshold concentration is at least 0.1 nM, at least 0.5 nM, at least 1 nM, at least 5 nM, at least 10 nM, at least 100 nM, at least 500 nM, at least 1 μM, or at least 5 μM. In some instances, when the target molecule is cortisol, the threshold concentration is less than 0.1 nM. In some instances, when the target molecule is cortisol, the threshold concentration is greater than 5 μM. When said target molecule is histamine, said threshold concentration is 0.1 nM, at least 0.5 nM, at least 1 nM, at least 5 nM, at least 10 nM, at least 100 nM, at least 500 nM, at least 1 μM, or at least 5 μM. In some instances, when the target molecule is histamine, the threshold concentration is less than 0.1 nM. In some instances, when the target molecule is histamine, the threshold concentration is greater than 5 μM.

The response factor can comprise any molecule that contributes to the use of the biosensing compositions described herein. For instance, the response factor can be any factor that contributes to reducing the levels and/or activity of a target molecule, treating a disease or disorder, or a sign or symptom of a disease or disorder, and/or maintaining a healthy biological function within a tissue of a subject. A healthy biological function will be depending upon the specific tissue. In some instances, maintaining a healthy biological function in a tissue comprises binding, detecting, and/or reducing the levels of cortisol and/or histamine in said tissue, wherein said tissue comprises the skin or in the esophagus, nasal tissue, mouth, vaginal tissue, lungs, stomach, small intestines, large intestines, digestive tract, or gastrointestinal tract of said subject. In some instances, the response factor comprises a postbiotic compound. Any postbiotic compound described herein can be produced and/or secreted by the engineered yeast cells disclosed herein.

The method of manufacturing a biosensing composition can further comprise formulating the biosensing compositions into the form of a capsule, gel, softgel, paste, tablet, powder, gummy, spray, aerosol, lozenge, effervescent tablet, orodispersible film, topical cream, ointment, suppository, emulsion, transdermal patch, or liquid. The method can comprise formulating the biosensing composition into a nutritional supplement, nutritional whole food, drink, sports drink, coffee, tea, water, mineral water, soda, carbonated beverage, oral hydration drink, mineral water, soup, replacement food, nutritional bar, nutritional consumable, gummy, tablet, confectionery, fermented or unfermented milk-based product, yogurt product, milk-based powder, enteral nutritional product, fermented or unfermented cereal-based product, chocolate, milk, yogurt, cheese, ice cream, baby and infant formula, cereal, animal feed, protein shake, protein powder, functional water, juice blend, smoothie, ready-to-drink (RTD) beverage, non-dairy milk, nut or seed butter, energy drink, functional shot, baked good, granola bar, snack bar, alcoholic beverage, frozen food, frozen meal, condiment, pasta, noodles, pet food, or pet treat. In some instances, the biosensing composition is formulated for administration to a plant or plurality of plants.

The following examples are offered by way of illustration and not by way of limitation.

### EXAMPLES

### Example 1: Yeast Transformation Protocol

Disclosed herein is a transformation procedure that is used to introduce polynucleotides encoding a molecule responsive element and response factor into yeast cells.

### Materials:

a. YPD
   10 g/L Yeast extract
   20 g/L Peptone
   20 g/L Dextrose (Glucose)
   Add water to 1 L
   Autoclave to sterilise
b. Sterile ddH2O
c. 50% w/v PEG 3350
   Add 50 g of PEG 3350 to a suitable flask, crush PEG to reduce particle size (this aids dissolution).
   Add 40 ml of ddH2O along with a suitable stir bar and begin mixing
   Once mostly dissolved (can be done overnight) measure volume in a clean cylinder.
RInce flask with 1-2 ml and add to cylinder.
   Top PEG 3350 solution volume off to 50 ml with ddH2O—Place para-film over mouth of flask and, holding file tightly to seal cylinder, invert the mixture to combine. Final volume is important here
   Transfer PEG to a small autoclave bottle, screw lid on tight to stop evaporation and autoclave to sterilise—PEG 3350 now ready to use. (NOTE—Though PEG is good for several months evaporation leads to increased PEG concentration and decreases transformation efficiency. If in doubt make fresh)
d. 1M LiAc
   Add 3.29 g of Lithium acetate powder (65.98 g mol-1) to 50 ml of ddH2O in a 50 ml Falcon tube.
   Vortex to dissolve powder and confirm 50 ml final volume.
   Filter sterilise (NOTE—Can also be autoclaved)
e. 2 mg/ml ssDNA Carrier DNA (Salmon Sperm DNA)
   Frozen 10 mg/ml stock tubes (Invitrogen CAT: AM9680) are used for preparing working solution. Dilute this 1/5 in ddH2O to obtain desired 2 mg/ml working solution.
Dilute enough such that you have 50 ul per transformation to be performed.
   ssDNA will be boiled on PCR machine shortly before transformation mix is prepared
f. 1 ug of transformation DNA (i.e Plasmid DNA)
   Resuspend transforming DNA in 34 ul of sterile ddH2O for each transformation
g. Selective plates
   Plate or media that will select for cells that have taken up or intergrated the desired markers.

### Method

### Day 1

1. Inoculate a 5 ml YPD liquid culture with yeast strain to be transformed by either picking a colony from a freshly streaked plate or by scraping cells from a −80 C glycerol stock.

2. Grow culture overnight @30 C with shaking

### Day 2

3. Dilute overnight yeast culture to 0.1 OD600 in fresh YPD—5 ml culture per transformation to be performed

4. Incubate culture(s) @30 C with shaking until OD600 reaches 0.8. This is strain dependent but usually 4-6 hours.

5. Pellet cell cultures on large centrifuge (5 min @4000 rpm)

6. While centrifuging Prepare 0.1M LiAc wash solution from 1M stock. (need 2 ml per transformation). Boils ssDNA on PCR machine (95 C for 5 minutes—hold @4 C till ready)

7. Pour off YPD growth media and resuspend the cell pellet (by vortexing) in 1 ml of sterile ddH2O. Transfer to fresh eppendorf and repellet cells in micro-centrifuge (10K rpm—1 minute)

8. Repeat cells wash with 1 ml of ddH20

9. Wash cells twice with 1 mL 0.1M LiAc (can vortex). After final wash carefully remove as much liquid form cell pellet as possible with vacuum pump.

10. Add the following components to the cell pellet in the order listed:
PEG3350 50% w/v→240 uL
1M LiAc→36 uL
Boiled ssDNA→50 uL
Transformation DNA→34 uL
Total→360 uL

11. Resuspend cell pellet gently using clean pipette tip then vortex briefly to evenly distribute cells

12. Incubate tubes @30 C for 30 minutes, then transfer to a 42 C water bath for 30-45 minutes.

13. Spin down tubes for 30 s at max sped (~13K rpm) and removed transformation mix with either a 1 ml pipette or vacuum.

14. Wash cell pellet once (carefully/gently) with 1 ml sterile ddH2O. After repellet and wash removal either:
a) If the selection of successful transformation involves an auxotrophic marker (ie. URA3, LEU2 etc) gently resuspened the pellet in 1 ml of sterile ddH2O and plate 200 ul (20%) onto the appropriate amino acid drop out media
b) If the selection uses a drug resistance marker (ie KanMX4 for G418 resistance selection) gently resuspend the cell pellet in 1 ml of YPD and recover the cells for ~1 h @30 C with shaking. Once recovered, wash the cells with 1 ml of sterile ddH2O to remove leftover media and resuspend in 1 ml of fresh ddH2O. 200 ul of cells can then be spread on selective plate—i.e YPD+G418 for KanMX4 selection.

### Example 2: Cortisol Biosensing Composition

The cortisol biosensor is a two-component gene circuit involving a human GC receptor. The first component is a GC receptor, which functions as the input/detection module of the biosensor. The GC receptor is a native human protein (SEQ ID NO: 1) that binds to and/or interacts with cortisol. The second component is an expression cassette, which functions as the output/signal module of the biosensor. A GFP expression cassette was programmed to be responsive to a GC receptor (i.e., interacting with cortisol) by placing a hormone response element (HRE) (SEQ ID NO: 11) upstream of the GFP cDNA (i.e., operably linking the HRE polynucleotide to the nucleotide sequence encoding GFP). cDNA for both GC receptor and HRE-GFP expression cassette were synthesized and sequences validated via Sanger sequencing.

To link production of a response factor to detection of cortisol, a polynucleotide encoding the response factor can operably linked to the HRE nucleotide sequence. The production of any response factor is programmed to be responsive to GC receptor (i.e., upon interaction with cortisol) by operably linking the HRE nucleotide sequence to the polynucleotide encoding the response factor.

### Example 3: Production of an Engineered Yeast Comprising a Cortisol Biosensing Composition

In the first generation of the S. boulardii cortisol biosensor, GC receptor polynucleotide and HRE-GFP expression construct were integrated into the S. boulardii genome. The homothallic switching endonuclease (HO) loci was chosen for its safe harbor characteristics. Since S. boulardii is a diploid organism, both the GC receptor polynucleotide and HRE-GFP expression construct were concurrently integrated at the HO loci on separate chromosomes. The GC receptor polynucleotide and HRE-GFP expression cassette were designed to harbor different antibiotic selection markers, such that S. boulardii strains comprising both modules was resistant to G418 (geneticin) and Nourseothricin (ClonNAT).

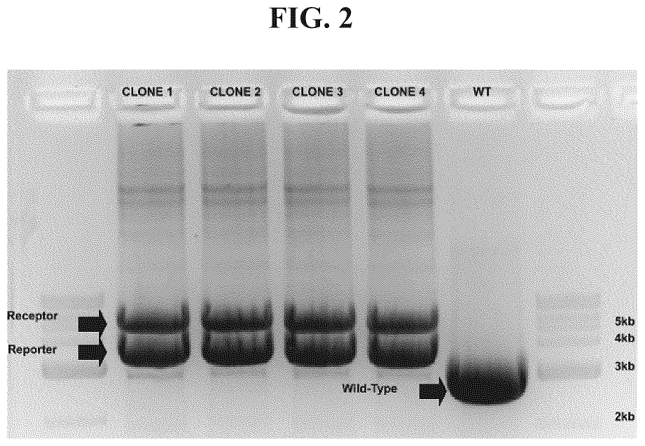

After two sequential transformations and selections, S. boulardii clones were screened using PCR primers targeting the HO endonuclease loci. The presence of PCR products consistent with an integrated GC receptor polynucleotide and HRE-GFP expression construct was confirmed in four tested clones, compared to the HO loci in wildtype S. boulardii, where only a single PCR product consistent with an intact HO endonuclease gene was detected, as depicted in FIG. 2.

Cortisol sensing assays were performed using the S. boulardii strain comprising the cortisol biosensor (GC receptor polynucleotide and HRE-GFP expression construct) and wildtype S. boulardii. Briefly, strains were inoculated into 5 mL of synthetic complete media containing G418 and nourseothricin to select for integration of both the GC receptor and HRE-GFP component. Strains were grown to saturation overnight at 30 C. The following day, cultures were diluted to an OD600 of 0.04 and 1 mL of diluted culture was transferred to wells in a 96-deep well block. A titration series of cortisol concentrations was mixed in DMSO and added to each well (including DMSO only controls). Four technical replicates were performed for each strain at each cortisol concentration. Following assay set up, deep well blocks were incubated for approximately 24 hours at 30 C with shaking. After cortisol treatment, 200 uL of test culture was transferred to a 96-well plate for green fluorescence measurements. To determine the green fluorescence measurements, an average value for the four technical replicates was calculated, and the average value for DMSO only negative control was subtracted from each experimental value.

In this pilot experiment, no green fluorescence signal was observed in a dose-response manner (data not shown). Unexpectedly, strains treated with higher levels of cortisol exhibited less green fluorescence signal compared to DMSO controls. Furthermore, recombinant strains only exhibited positive green fluorescence signals at low cortisol concentrations of 15 and 7.8 uM, the two lowest concentrations tested. This result suggested that the biosensor was not functioning when integrated into the yeast genome, as examined this assay.

Expression of a gene located on the genome of a microorganism may differ from that of expression from a non-chromosomal polynucleotide (e.g., a plasmid), for instance, in copy number and regulation, which may impact cellular physiology. It would be understood that copy number of a gene within a cell can be correlated with expression levels of the gene product, and that copy number can be increased by introducing the gene into a cell on a multi-copy polynucleotide such as a plasmid. While chromosomal genes are typically present in single copy and expressed under native regulatory controls, plasmids, including high-copy plasmids, introduce multiple gene copies and often employ strong, synthetic promoters. Thus, expression of a gene from a plasmid may results in an increase in transcription and translation rates compared to the chromosome. While this can boost product yield, cell physiology may also be impacted, e.g., it may impose metabolic stress, increase potential protein misfolding, and decrease overall cell viability. These differences have significant implications for the functionality of expressed products and the choice of expression system, depending on whether the goal is high-yield production or long-term stability. Because such gene circuits may not predictably perform as effectively or efficiently across microorganisms, they must be tested in a particular host. Without wishing to be bound by any theory, it is possible that, less than optimal concentrations of signal input/output elements may resulted in a lack of signal.

The GC receptor polynucleotide and HRE-GFP expression construct described above were cloned into high-copy yeast plasmids. Plasmids are maintained in cells at a higher copy number compared to genetically integrated elements. Without wishing to be bound by theory, it is possible that high-copy number of the GC receptor polynucleotide and HRE-GFP expression cassette within the yeast cell may result in more amounts within the cell. Both the GC receptor polynucleotide and HRE-GFP expression construct were cloned into a single G418-resistant plasmid, which was transformed into S. boulardii. Two versions of the plasmid, differing in the relative positioning of the GC receptor polynucleotide and HRE-GFP expression construct within the plasmid. Specifically, the positioning of these elements was swapped.

An S. boulardii strain transformed with a single plasmid comprising both the GC recetpro and HRE-GFP expression construct was grown to saturation overnight. The following day, cultures were diluted to a OD600 of 0.8. The diluted culture was transferred to a 96-well plate with 200 uL of culture in each well. A titration series of cortisol concentrations were added to cultures to a final concentration of 500, 250, 125, 62.5, 0.625 and 0 uM, as depicted in Table 1. Plates were incubated at 30 degrees Celsius with shaking and green fluorescence measurement was taken every 0.5 hours for 5 hours on a BioTek Synergy plate reader.

TABLE 1
Cortisol Concentrations Tested.

| Dilution | Stock Concentration | Volume in 1 ml | Cortisol Concentration |
|---|---|---|---|
| # | [mM] | culture | in culture [μM] |
| 0 | 50 | 20 | 1000 |
| 1 | 25 | 20 | 500 |
| 2 | 12.5 | 20 | 250 |
| 3 | 6.25 | 20 | 125 |
| 4 | 3.125 | 20 | 62.5 |
| 5 | 1.5625 | 20 | 31.25 |
| 6 | 0.78125 | 20 | 15.625 |
| 7 | 0.390625 | 20 | 7.8125 |
| DM | 0 | 20 | 0 |

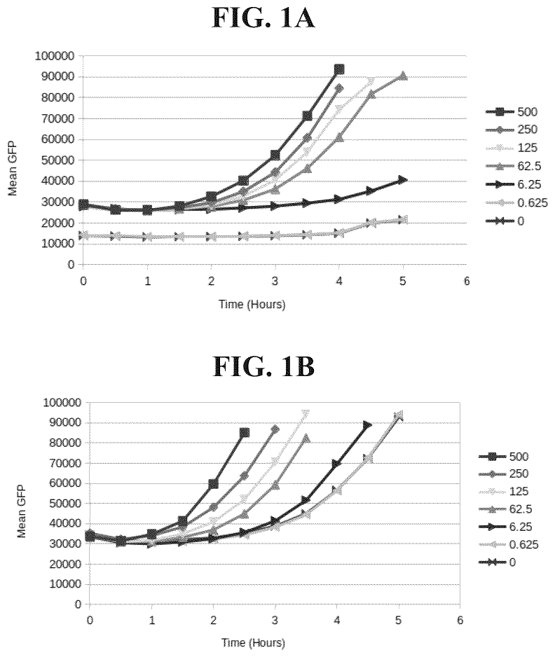

In both S. boulardii strains transformed with a cortisol biosensor encoding plasmid, a dose-response relationship between green fluorescence signal and cortisol concentration was observed (FIG. 1A and FIG. 1i). These data strongly suggest that these S. boulardii strains are capable of sensing cortisol in the environment. Noticeably, the rate of signal appearance and background signal differed between the two strains (compare FIG. 1A and FIG. 1B). Strain version 2 exhibits green fluorescence signal at 3 hours even in the absence of cortisol (FIG. 1B). As the two strains differ only with respect to the relative positioning of the GC receptor polynucleotide and HRE-GFP expression construct within the plasmid, these data suggest that the placement of an element of the cortisol two-component biosensor on the plasmid can impact functionality. Without wishing to be bound by any theory, it is possible that positioning of the nucleotide sequences on a plasmid may influence gene expression and/or mRNA stability, and thus protein levels, of biosensing components, resulting in differences in response factor expression and/or protein levels.

In conclusion, the present study demonstrated a recombinant S. boulardii strain comprising a cortisol biosensor, i.e., a GC receptor polynucleotide and a HRE-GFP expression construct can function as a cortisol biosensor. In vitro, the limit of detection is between 6.25 uM to 62.5 uM. Green fluorescence signals can be detectable as quickly as 1.5 hours. Further optimization of the gene circuit can be performed to improve the sensitivity and signal output of the biosensor for probiotic usage.

### Example 4: Production of an Engineered Yeast for Modulation of Cortisol, Intermediate Molecules, and/or Downstream Effects of Elevated Cortisol

A yeast cell is engineered to detect cortisol and produce a response factor as described in Example 3. The engineered yeast cell produces any response factor in response to the detection of cortisol (i.e., interaction of the GC receptor with cortisol). To produce a response factor, an expression cassette comprising a polynucleotide encoding a response factor is operably linked to an HRE element, whereby expression of the response factor is activated via signal transduction following interaction of the GC receptor with cortisol. A polynucleotide encoding the GC receptor (i.e., GC receptor polynucleotide) and HRE-linked response factor expression cassette is introduced into the yeast cell through a transformation process as described above. Any process for introduction of a polynucleotide into yeast is used. The gene cassette can be located on a plasmid, such as a high-copy plasmid.

Interaction of human GC receptor with cortisol results in production of the response factor, which is any molecule that is to be produced in response to elevated levels of cortisol. In some instances, the response factor directly modulates levels of cortisol. In some instances, the response factor directly modulates the levels of at least a second molecule that is elevated in a tissue comprising elevated cortisol levels. The response factor interacts with cortisol, or intermediate molecules that can impact cortisol, at a molecular level to result in degradation, inhibition, or sequester of cortisol, inhibit the GC receptor, or modulate downstream effects of the elevated cortisol levels. Downstream effects of elevated cortisol levels include inflammation, stress, and other conditions described elsewhere herein.

### Example 5: Use of an Engineered Yeast as a Cortisol Biosensor to Detect Cortisol in or on the Tissue of a Subject

The yeast cell is engineered to detect and respond to cortisol by producing a response factor. The response factor modulates cortisol levels and/or the levels of a second molecule (i.e., a molecule that is not cortisol). In some instances, the engineered yeast produces a response factor that decreases the levels of a molecule that is not cortisol in response to cortisol levels detected above a threshold concentration. Any method of administration is used to effectively deliver the engineered yeast to the tissue. For example, the engineered yeast is administered to the gastrointestinal tract of the subject. The engineered yeast is orally administered to a subject such that the yeast is localized within the gastrointestinal tract of the subject. The engineered yeast cells are recovered from the gastrointestinal tract of the subject by taking a tissue sample from the GI tract. The engineered yeast cell can also be detected by imaging or sequencing technologies available to a skilled artisan for detecting the present of a microorganism within the gastrointestinal tract of a subject. In another specific embodiment, the engineered yeast is topically administered on the skin. In some instances, the yeast cell is Saccharomyces boulardii or Saccharomyces cerevisiae.

The tissue (e.g., gastrointestinal tract or skin) can comprise cortisol at varying levels. Within the tissue (e.g., gastrointestinal tract or skin), the engineered yeast detects the presence of cortisol via interaction of cortisol with the GC receptor. In particular, detection occurs when the cortisol is at a concentration of between at least 0.1 nM and at least 5 μM. The yeast increases production of the response factor, which is produced when the cortisol concentration remains and/or is maintained at or above the effective, threshold concentration. Once the cortisol level is below the effective, threshold concentration, the production of the response factor ceases, as the gene is no longer activated by the GC receptor. When engineered yeast is administered as a probiotic, the yeast cells are capable of colonizing in or on a tissue (e.g., gastrointestinal tract or skin) of the subject, such that the engineered yeast are recovered from the tissue of the subject for at least one day, at least one week, at least one month, or at least one year. The yeast is engineered to produce an response factor that reduces the levels of and/or reduces enzymatic activity of cortisol and/or at least a second molecule (i.e., a molecule that is not cortisol). The response factor is released into the environment, through secretion from the yeast, where the response factor can come into contact with the cortisol and/or at least a second molecule. Upon contact, the response factor can reduce levels of and can increase degradation of cortisol and/or an at least second molecule. The response factor can also inhibit enzymatic activity of cortisol or can inhibit interaction of cortisol and/or a second molecule.

### Example 6: Histamine Biosensing Composition

The histamine biosensor is a multi-gene circuit based on G protein coupled receptor (GPCR) signaling. To generate a recombinant yeast cell capable of sensing histamine, a histamine biosensing circuit was constructed comprising a human histamine receptor (hHRH) polynucleotide (i.e, a polynucleotide encoding a hHRH) encoding a hHRH polypeptide (SEQ ID NO: 3) that was codon-optimized for expression in yeast (SEQ ID NO: 4), and a GFP reporter expression construct responsive to the Ste12 transcription factor. Ste12 is a transcription factor that is responsive to GPCR signaling.

To enable the histamine biosensing construct to function in yeast, a modified GPA1 protein (SEQ ID NO: 8) capable of interacting with hHRH was generated. A modification was introduced into the C-terminal end of native GPA1 (i.e., amino acid substitution of residues at positions 468-472 compared to native GPA1 protein sequence (SEQ ID NO: 10): K468E, I469Y, G470N, I471L, and I472V). Interaction of hHRH with the modified GPA1 protein upon interaction with histamine activates the signaling pathway and expression of the response factor operably linked to Ste12-driven promoter, such as the FIG. 1 promoter of Saccharomyces cerevisiae. The use of Ste12 controlling reporter gene expression from either of the FUS1, FUS2 and FIG. 1 promoters remains the most common design for analyzing GPCR signaling in yeast (Trueheart, Boeke and Fink 1987; Trueheart and Fink 1989; Muller et al. 2003; Alvaro and Thorner 2016), though it is important to limit the expression strength of Ste12 in order not to impair cell growth (Shaw et al. 2019) (see Lengger B, Jensen M K. Engineering G protein-coupled receptor signalling in yeast for biotechnological and medical purposes. FEMS Yeast Res. 2020 Feb. 1; 20(1):foz087, the contents of which are incorporated herein in their entirety). cDNA constructs comprising a polynucleotide encoding the hHRH operably linked to the GAP promoter, and polynucleotide encoding GFP operably linked to the FIG. 1 promoter were synthesized and validated by sequencing.

To link production of a response factor to detection of histamine, a polynucleotide encoding a response factor can be operably linked to the Ste12-driven promoter. The production of any response factor is responsive to hHRH interaction with histamine by operably linking the Ste12-driven promoter polynucleotide to the polynucleotide encoding the response factor.

### Example 7: Production of an Engineered Yeast Cell Comprising a Histamine Biosensing Composition

In S. boulardii, GPCRs bind to their natural ligands and induce a cascade of MAP kinase signaling, ultimately leading to transcriptional activation of reporter genes. In humans, histamine is a natural ligand for human GPCRs, which can activate GPCR signaling in human cells. Thus, it was hypothesized that S. boulardii GPCR signaling can be subverted to detect histamine if S. boulardii cells are programmed to express histamine binding GPCRs. Engineering of a S. boulardii yeast strain comprising the histamine biosensing circuit as described in Example 6 was performed. To limit native GPCR signaling, such as signaling that is non-specific for the detection of histamine, S. boulardii comprising a full deletion of the native GPCR signaling genes STE2, FAR1, SST2 and MAT-A was generated. These modifications are essential in this approach to minimize the chance that native S. boulardii GPCR signaling activates the synthetic GFP reporter.

Methods of engineering yeast via CRISPR would be full within the capabilities of one having ordinary skill in the art. Multiple attempts to generate S. boulardii strains comprising homogenous knockouts of genes indicated above were unsuccessful. Thus, a similar approach was attempted in S. cerevisiae, a yeast lab strain that is more amenable to gene manipulation. S. cerevisiae, although not a common probiotic, is 99% genetically identical to S. boulardii.

A strain of S. cerevisiae with the required gene knockouts and edits (i.e., full deletion of the native GPCR signaling genes STE2, FAR1, SST2 and MAT-A; and introduction of a genetic alteration in the C-terminal end of the native GPA1, as described in Example 6.) was generated. This recombinant yeast strain comprises a polynucleotide sequence (SEQ ID NO: 7) encoding for a modified GPA1 protein (SEQ ID NO: 8) at the native GPA1 locus on the chromosome that functions with the recombinant histamine receptor, as well as a hHRH and a GFP reporter expression construct that is responsive to the GPCR signaling cascade (i.e., operably linked to a Ste12-driven promoter), introduced on two separate plasmids. The resulting strain (BY-HS-001), harboring 5 genetic changes compared to native yeast, grew noticeably slower compared to wildtype S. cerevisiae. All changes were validated by PCR analysis of the genome. BY-HS-001 was transformed with two plasmids comprising a hHRH polynucleotide and Ste12-GFP reporter expression construct for histamine sensing assays. Different versions of the hHRH and GFP reporter plasmids were generated having various selection markers.

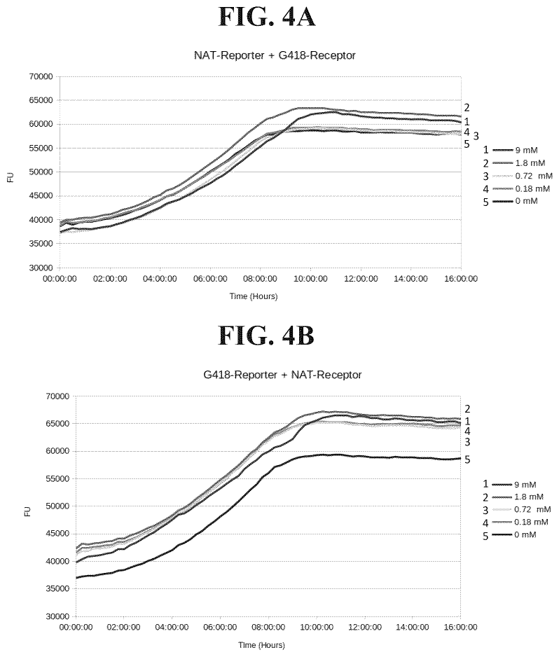

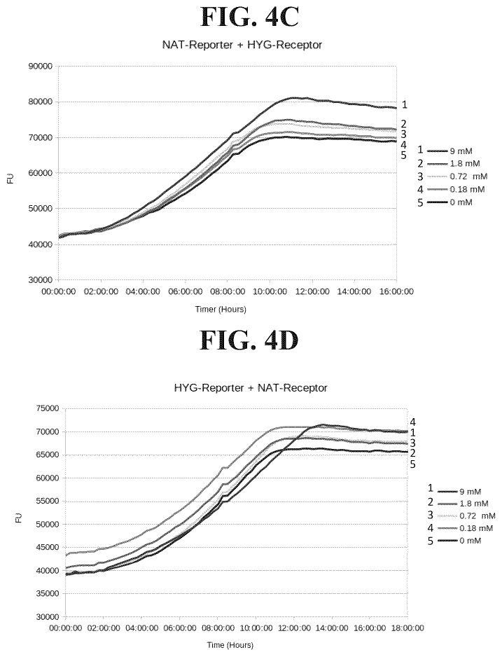

BY-HS-001 transformed with various plasmid combinations of the hHRH and GFP reporter (as identified in FIGS. 4A-4D) was grown to saturation overnight. The following day, cultures were diluted 1 in 10 and 100 uL of the diluted culture was transferred to a 96-well plate. Histamine was added to the strains at a final concentration of 9 mM, 1.8 mM, 0.72 mM, 0.18 mM and 0 mM in a total experimental volume of 200 uL. Strains were monitored for 18 hours in a BioTek Synergy plate reader with green fluorescence measurement taken every 15 minutes (FIGS. 4A-4D).

As depicted in FIGS. 4A-4D, four combinations of two plasmids comprising a hHRH polynucleotide and Ste-12 GFP reporter expression construct, wherein the two plasmids have different antibiotic resistance markers (Geneticin, “G418”; Nourseothricin, “NAT”; Hygromycin, “HYG”), lacked a detectable dose-dependent response signal in green fluorescence (“FU”; FIGS. 4A-4D) in any combination of plasmids. Unexpectedly, a fluorescence signal was detected in the absence of histamine (see “0 mM”; FIGS. 4A-4D), suggesting that the histamine sensing pathway as being auto-activated in cells.

A second study was performed using BY-HS-001 (having a hHRH polynucleotide and Ste12-GFP reporter expression construct on two plasmids) and a control strain having a Ste12-GFP reporter only (no hHRH). Also included was a modified S. cerevisiae control strain having a modified histamine receptor responsive GPA1 G-protein (SEQ ID NO: 8). This wildtype strain does not contain any histamine sensing GPCR pathways. Strains were grown for 18 hours at 30 C in the presence of different histamine concentrations. Growth was conducted in 200 uL in a 96-well plate. After 18 hours, green fluorescence measurements were made on a BioTek Synergy plate reader.

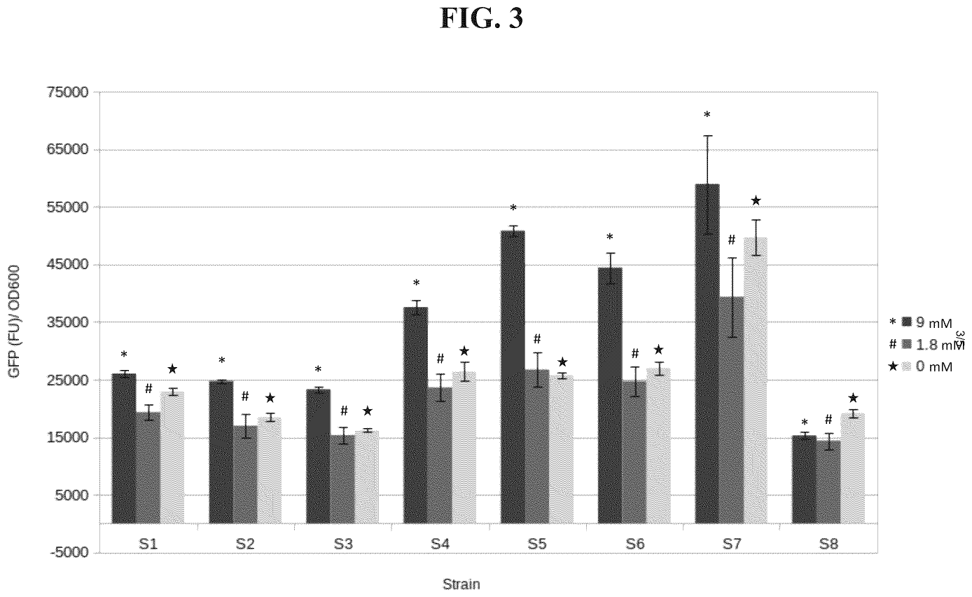

Interestingly, in the presence of 9 mM histamine, an increased green fluorescence signal was observed in all S. cerevisiae strains having a hHRH polynucleotide and Ste12-GFP reporter expression construct (S4 to S7) compared to control S. cerevisiae strains haivng only a Ste12-GFP reporter expression construct (S1 to S3) and wildtype S. cerevisiae (S8). These data indicate that the highest concentration of histamine tested does lead to higher levels of activation of the Ste12-driven response factor, suggesting that a certain concentration of histamine is required to activate the signaling pathway and/or downstream expression, as lower concentrations (i.e., 1.8 mM) showed florescence levels below the limit of detection (FIG. 3). This also indicates that both the hHRH polypeptide and modified GPA1 protein are required for detection and activation in response to histamine. Secondly, strains S1 to S3 do exhibit higher green fluorescence signal at 9 mM histamine compared to wildtype S8, suggesting that GPCR signaling is activated in yeast strains not having a hHRH polypeptide. Without wishing to be bound by theory, one reasonable explanation for this result is that histamine can non-specifically bind receptors present within the tested yeast cells at the concentration of histamine tested resulting in downstream activation.

In conclusion, S. cerevisiae with a putative histamine biosensing circuit was generated.

### Example 8: Production of an Engineered Yeast for Modulation of Histamine, Intermediate Molecules, and/or Downstream Effects of Elevated Histamine

A yeast cell is engineered to detect histamine and produce a response factor using the histamine receptor as described in Examples 6 and 7. The engineered yeast cell produces any response factor in response to the detection of histamine (i.e., interaction of the human histamine receptor with histamine). To produce an response factor, a gene cassette comprising a polynucleotide encoding the response factor is operably linked to a promoter responsive to the transcription factor STE12 (e.g., FIG. 1 promoter), whereby expression of said response factor is activated via signal transduction by STE12 following GPCR signaling. A polynucleotide encoding the human histamine receptor and response factor is introduced into the yeast cell through a transformation process as described above. Any process for introduction of a polynucleotide into yeast is used. The gene cassette can be located on a plasmid, such as a high-copy plasmid.

Interaction of human histamine receptor with histamine results in production of the response factor, which is any molecule that is to be produced in response to elevated levels of histamine. In some instances, the response factor directly modulates levels of histamine. In some instances, the response factor directly modulates the levels of at least a second molecule that is elevated in a tissue comprising elevated histamine levels. The response factor interacts with histamine, or intermediate molecules that can impact histamine, at a molecular level to result in degradation, inhibition, or sequester of histamine, inhibit the GPCR, or modulate downstream effects of the elevated histamine levels.

### Example 9: Use of an Engineered Yeast as a Histamine Biosensor to Detect Histamine in or on Tissue of a Subject

The yeast cell is engineered to detect and respond to histamine by producing a response factor. The response factor modulates histamine levels and/or the levels of a second molecule (i.e., a molecule that is not histamine). In some instances, the engineered yeast produces a response factor that decreases the levels of a molecule that is not histamine in response to histamine levels detected above a threshold concentration. Any method of administration is used to effectively deliver the engineered yeast to the tissue. For example, the engineered yeast is administered to the gastrointestinal tract of the subject. The engineered yeast is orally administered to a subject such that the yeast is localized within the gastrointestinal tract of the subject. The engineered yeast cells are recovered from the gastrointestinal tract of the subject by taking a tissue sample from the GI tract. The engineered yeast cell can also be detected by imaging or sequencing technologies available to a skilled artisan for detecting the present of a microorganism within the gastrointestinal tract of a subject. In another specific embodiment, the engineered yeast is topically administered on the skin. In some instances, the yeast cell is Saccharomyces boulardii or Saccharomyces cerevisiae.

The tissue (e.g., gastrointestinal tract or skin) can comprise cortisol at varying levels of histamine. tissue (e.g., gastrointestinal tract or skin), the engineered yeast detects the presence of histamine via interaction of histamine with the GPCR. In particular, detection occurs when the histamine is at a concentration of between at least 0.1 nM and at least 5 μM. The yeast increases production of the response factor, which is produced when the histamine concentration remains and/or is maintained at or above the threshold concentration. Once the histamine level is below the threshold concentration, the production of the response factor ceases, as the gene is no longer activated by the GCPR. When engineered yeast is administered as a probiotic, the yeast cells are capable of colonizing in or on a tissue (e.g., gastrointestinal tract or skin) of the subject, such that the engineered yeast are recovered from the tissue of the subject for at least one day, at least one week, at least one month, or at least one year. The yeast is engineered to produce a response factor that reduces the levels of and/or reduces enzymatic activity of histamine and/or at least a second molecule (i.e., a molecule that is not histamine). The response factor is released into the environment, through secretion from the yeast cell, where the response factor can come into contact with histamine molecule and/or at least a second molecule. Upon contact, the response factor can interact with and can increase degradation of histamine and/or at least a second molecule. The response factor can also inhibit enzymatic activity of histamine and/or at least a second molecule or can inhibit interaction of histamine and/or a second molecule.

TABLE 2
Sequence Description Table.

| SEQ ID | |
|---|---|
| NO: | Description |
| 1 | Amino acid sequence encoding human GC receptor |
| | (NCBI Accession No. NP_000167.1) |
| 2 | Codon-optimized nucleotide sequence encoding human |
| | GC receptor of SEQ ID NO: 1 |
| 3 | Amino acid sequence encoding human histamine H1 receptor |
| | (NCBI Accession No. NP_000852) |
| 4 | Codon-optimized nucleotide sequence encoding human |
| | histamine H1 receptor of SEQ ID NO: 3 |
| 5 | Nucleotide sequence of the response element of the GC |
| | response element/promoter of SEQ ID NO: 11 |
| 6 | Nucleotide sequence of the minimal promoter of the GC |
| | response element/promoter of SEQ ID NO: 11 |
| 7 | Nucleotide sequence encoding modified yeast GPA1 |
| | nucleotide sequence |
| 8 | Amino acid sequence encoding modified yeast GPA1 protein |
| 9 | Nucleotide sequence of native GPA1 gene in S. cerevisiae |
| | (NCBI Accession No. M15867) |
| 10 | Amino acid sequence of native GPA1 protein S. cerevisiae |
| | (NCBI Accession No. NP_011868.1) |
| 11 | Nucleotide sequence of GC response element/promoter |
| 12 | GAP promoter sequence |

The section headings used herein are for organizational purposes only and are not to be construed as limiting the subject matter described in any way.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

While various aspects of the invention are described herein, it is not intended that the invention be limited by any particular aspect. On the contrary, the invention encompasses various alternatives, modifications, and equivalents, as will be appreciated by those of skill in the art. Furthermore, where feasible, any of the aspects disclosed herein may be combined with each other (e.g., the feature according to one aspect may be added to the features of another aspect or replace an equivalent feature of another aspect) or with features that are well known in the art, unless indicated otherwise by context.

That which is claimed:

1. A biosensing composition for detecting a target molecule in a subject, said composition comprising a plurality of engineered yeast cells comprising:
a) a molecule responsive element comprising a binding domain that interacts with said target molecule, and/or a polynucleotide comprising a nucleotide sequence encoding said molecule responsive element; and
b) a nucleic acid molecule comprising a polynucleotide comprising a nucleotide sequence encoding a response factor,
whereby said interaction of said target molecule with said binding domain results in production and/or secretion of said response factor in said engineered yeast cells.

2. The biosensing composition of claim 1, wherein said molecule responsive element comprises a polypeptide.

3. The biosensing composition of claim 2, wherein said polypeptide comprises an amino acid sequence comprising said binding domain.

4. The biosensing composition of any one of claims 1-3, wherein said molecule responsive element comprises a nucleic acid molecule.

5. The biosensing composition of claim 2 or 3, wherein said polypeptide comprises a protein, such as a fusion protein, a transmembrane protein, a receptor, and/or a functional fragment or variant thereof.

6. The biosensing composition of claim 5, wherein said receptor comprises a human glucocorticosteroid (GC) receptor, or a functional fragment or variant thereof having at least 80%, at least 85%, at least 90%, at least 95%, or at least 99% sequence identity to a human glucocorticosteroid (GC) receptor.

7. The biosensing composition of claim 6, wherein said human glucocorticosteroid (GC) receptor comprises an amino acid sequence set forth as SEQ ID NO: 1, or a functional fragment or variant thereof having at least 80%, at least 85%, at least 90%, at least 95%, or at least 99% sequence identity to the amino acid sequence set forth as SEQ ID NO: 1; and/or
wherein said human glucocorticosteroid (GC) receptor is encoded by a nucleotide sequence set forth as SEQ ID NO: 2, or a variant thereof having at least 80%, at least 85%, at least 90%, at least 95%, or at least 99% sequence identity to the nucleotide sequence set forth as SEQ ID NO: 2.

8. The biosensing composition of claim 5, wherein said receptor comprises a G-protein coupled receptor (GPCR) protein, or a functional fragment or variant thereof having at least 80%, at least 85%, at least 90%, at least 95%, or at least 99% sequence identity to a G-protein coupled receptor (GPCR) protein.

9. The biosensing composition of claim 8, wherein said G-protein coupled receptor (GPCR) protein is a human G-protein coupled receptor (GPCR) protein.

10. The biosensing composition of claim 8 or 9, wherein said G-protein coupled receptor (GPCR) protein, or functional fragment or variant thereof, comprises a histamine binding domain, such as a histamine binding ectodomain.

11. The biosensing composition of claim 9 or 10, wherein said human G-protein coupled receptor (GPCR) protein comprises an amino acid sequence set forth as SEQ ID NO: 3, or a functional fragment or variant thereof having at least 80%, at least 85%, at least 90%, at least 95%, or at least 99% sequence identity to the amino acid sequence set forth as SEQ ID NO: 3; and/or
wherein said human G-protein coupled receptor (GPCR) protein is encoded by a nucleotide sequence set forth as SEQ ID NO: 4, or a variant thereof having at least 80%, at least 85%, at least 90%, at least 95%, or at least 99% sequence identity to the nucleotide sequence set forth as SEQ ID NO: 4.

12. The biosensing composition of any one of claims 1-11, wherein said yeast cells further comprise a polypeptide comprising an amino acid sequence encoding a modified yeast GPA1 protein, or a polynucleotide comprising a nucleotide sequence encoding a modified yeast GPA1 protein,
wherein said polypeptide:
a) comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10;
b) comprises an amino acid sequence corresponding with residues 1-467 of SEQ ID NO: 10;
c) comprises an amino acid sequence comprising one or more than one amino acid substitution compared to SEQ ID NO: 10, wherein said one or more than one amino acid substitution is selected from the following: K468E, 1469Y, G470N, 1471L, and 1472V;
d) comprises an amino acid sequence comprising the following amino acid substitutions: K468E, I469Y, G470N, I471L, and I472V, compared to SEQ ID NO: 10; and/or
e) comprises an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8; and/or
wherein said polynucleotide comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

13. The biosensing composition of any one of claims 1-12, wherein said polynucleotide comprising a nucleotide sequence encoding a response factor is operably fused to an activation domain.

14. The biosensing composition of claim 13, wherein said activation domain comprises a promoter, such as a Ste-12-activated or a human glucocorticosteroid response element (HRE)-activated promoter.

15. The biosensing composition of claim 14, wherein said response factor is produced upon interaction of STE12 or GC receptor to said promoter.

16. The biosensing composition of any one of claims 1-15, wherein said engineered yeast comprises mutation and/or deletion in at least one of the following genes: STE2, FAR1, SST2, and MTA-A.

17. The biosensing composition of any one of claims 1-16, wherein said nucleotide sequence encoding said molecule responsive element is codon-optimized for expression in a eukaryotic cell, such as a yeast cell.

18. The biosensing composition of any one of claims 1-17, wherein said polynucleotide comprising a nucleotide sequence encoding said molecule responsive element and said polynucleotide encoding said response factor are comprised within a single nucleic acid molecule.

19. The biosensing composition of claim 18, wherein said nucleic acid molecule comprises a plasmid.

20. The biosensing composition of any one of claims 1-17, wherein said polynucleotide comprising a nucleotide sequence encoding said molecule responsive element and/or said polynucleotide encoding a response factor are integrated into the genome of said engineered yeast cells.

21. The biosensing composition of any one of claims 1-20, wherein said engineered yeast comprises Saccharomyces, such as Saccharomyces cerevisiae and/or Saccharomyces boulardii.

22. The biosensing composition of any one of claims 1-21, wherein said target molecule comprises a ligand, a protein, metabolite, a hormone, a nutrient, or small molecule, such as cortisol or histamine.

23. The biosensing composition of any one of claims 1-22, wherein said target molecule comprises cortisol or histamine.

24. The biosensing composition of any one of claims 1-23, wherein said engineered yeast cells produce an effective amount of said response factor in response to a threshold concentration of said target molecule.

25. The biosensing composition of claim 24, wherein when said target molecule is cortisol, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

26. The biosensing composition of claim 24, wherein when said target molecule is histamine, said threshold concentration is at least 0.001 nM, at least 0.01 nM, at least 0.1 nM, at least 0.5 nM, at least 1 nM, at least 5 nM, at least 10 nM, at least 100 nM, at least 500 nM, at least 1 μM, or at least 5 μM.

27. A probiotic comprising the biosensing composition of any one of claims 1-26.

28. The probiotic of claim 27, wherein said probiotic comprises a single species of microorganism.

29. The probiotic of claim 27 or 28, wherein said probiotic comprises at least two different species of microorganisms.

30. The probiotic of any one of claims 27-29, wherein said response factor comprises a postbiotic compound.

31. The probiotic of any one of claims 27-30, wherein said probiotic comprises a nutritional supplement.

32. The probiotic of any one of claims 27-31, wherein said probiotic is in the form of a capsule, gel, softgel, paste, tablet, powder, gummy, spray, aerosol, lozenge, effervescent tablet, orodispersible film, topical cream, ointment, suppository, emulsion, transdermal patch, or liquid.

33. The probiotic of any one of claims 27-32, wherein said probiotic is formulated into a nutritional whole food, drink, sports drink, coffee, tea, water, mineral water, soda, carbonated beverage, oral hydration drink, mineral water, soup, replacement food, nutritional bar, nutritional consumable, gummy, tablet, confectionery, fermented or unfermented milk-based product, yogurt product, milk-based powder, enteral nutritional product, fermented or unfermented cereal-based product, chocolate, milk, yogurt, cheese, ice cream, baby and infant formula, cereal, animal feed, protein shake, protein powder, functional water, juice blend, smoothie, ready-to-drink (RTD) beverage, non-dairy milk, nut or seed butter, energy drink, functional shot, baked good, granola bar, snack bar, alcoholic beverage, frozen food, frozen meal, condiment, pasta, noodles, pet food, or pet treat.

34. The biosensing composition of any one of claims 1-26 or the probiotic of any one of claims 27-33, wherein said biosensing composition or probiotic is formulated for administration to a plant or plurality of plants.

35. An engineered yeast cell comprising
a molecule responsive element comprising a binding domain that interacts with a target molecule, and/or a polynucleotide comprising a nucleotide sequence encoding said molecule responsive element; and
a nucleic acid molecule comprising a polynucleotide comprising a nucleotide sequence encoding a response factor.

36. The engineered yeast cell of claim 35, wherein said molecule responsive element comprises a polypeptide.

37. The engineered yeast cell of claim 36, wherein said polypeptide comprises an amino acid sequence comprising said binding domain.

38. The engineered yeast cell of any one of claims 35-37, wherein said molecule responsive element comprises a nucleic acid molecule.

39. The engineered yeast cell of any one of claims 36-38, wherein said polypeptide comprises a protein, such as a fusion protein, a transmembrane protein, a receptor, and/or a functional fragment or variant thereof.

40. The engineered yeast cell of claim 39, wherein said receptor comprises a human glucocorticosteroid (GC) receptor, or a functional fragment or variant thereof having at least 80%, at least 85%, at least 90%, at least 95%, or at least 99% sequence identity to a human glucocorticosteroid (GC) receptor.

41. The engineered yeast cell of claim 40, wherein said human glucocorticosteroid (GC) receptor comprises an amino acid sequence set forth as SEQ ID NO: 1, or a functional fragment or variant thereof having at least 80%, at least 85%, at least 90%, at least 95%, or at least 99% sequence identity to the amino acid sequence set forth as SEQ ID NO: 1; and/or
wherein said human glucocorticosteroid (GC) receptor is encoded by a nucleotide sequence set forth as SEQ ID NO: 2, or a variant thereof having at least 80%, at least 85%, at least 90%, at least 95%, or at least 99% sequence identity to the nucleotide sequence set forth as SEQ ID NO: 2.

42. The engineered yeast cell of claim 39, wherein said receptor comprises a G-protein coupled receptor (GPCR) protein, or a functional fragment or variant thereof having at least 80%, at least 85%, at least 90%, at least 95%, or at least 99% sequence identity to a G-protein coupled receptor (GPCR) protein.

43. The engineered yeast cell of claim 42, wherein said G-protein coupled receptor (GPCR) protein is a human G-protein coupled receptor (GPCR) protein.

44. The engineered yeast cell of claim 42 or 43, wherein said G-protein coupled receptor (GPCR) protein, or functional fragment or variant thereof, comprises a histamine binding domain, such as a histamine binding ectodomain.

45. The engineered yeast cell of any one of claims 42-44, wherein said human G-protein coupled receptor (GPCR) protein comprises an amino acid sequence set forth as SEQ ID NO: 3, or a functional fragment or variant thereof having at least 80%, at least 85%, at least 90%, at least 95%, or at least 99% sequence identity to the amino acid sequence set forth as SEQ ID NO: 3; and/or
wherein said human G-protein coupled receptor (GPCR) protein is encoded by a nucleotide sequence set forth as SEQ ID NO: 4, or a variant thereof having at least 80%, at least 85%, at least 90%, at least 95%, or at least 99% sequence identity to the nucleotide sequence set forth as SEQ ID NO: 4.

46. The engineered yeast cell of any one of claims 35-45, further comprising a polypeptide comprising an amino acid sequence encoding a modified yeast GPA1 protein, or a polynucleotide comprising a nucleotide sequence encoding a modified yeast GPA1 protein,
wherein said polypeptide:
a) comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10;
b) comprises an amino acid sequence corresponding with residues 1-467 of SEQ ID NO: 10;
c) comprises an amino acid sequence comprising one or more than one amino acid substitution compared to SEQ ID NO: 10, wherein said one or more than one amino acid substitution is selected from the following: K468E, 1469Y, G470N, 1471L, and 1472V;
d) comprises an amino acid sequence comprising the following amino acid substitutions: K468E, I469Y, G470N, I471L, and I472V, compared to SEQ ID NO: 10; and/or
e) comprises an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8; and/or
wherein said polynucleotide encoding a modified yeast GPA1 protein comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

47. The engineered yeast cell of any one of claims 35-46, wherein said polynucleotide is operably fused to an activation domain.

48. The engineered yeast cell of claim 47, wherein said activation domain comprises a promoter, such as a Ste-12-activated or a human glucocorticosteroid response element (HRE)-activated promoter.

49. The engineered yeast cell of claim 48, wherein the response factor is produced upon binding of STE12 or GC receptor to said promoter.

50. The engineered yeast cell of any one of claims 35-49, wherein said engineered yeast comprises mutation and/or deletion in at least one of the following genes: STE2, FAR1, SST2, and MTA-A.

51. The engineered yeast cell of any one of claims 35-50, wherein said nucleotide sequence encoding said molecule responsive element is codon-optimized for expression in a eukaryotic cell, such as a yeast cell.

52. The engineered yeast cell of any one of claims 35-51, wherein said polynucleotide comprising a nucleotide sequence encoding said molecule responsive element and said polynucleotide encoding a response factor are within a single nucleic acid molecule.

53. The engineered yeast cell of claim 52, wherein said nucleic acid molecule comprises a plasmid.

54. The engineered yeast cell of any one of claims 35-52, wherein said polynucleotide comprising a nucleotide sequence encoding said molecule responsive element and/or said polynucleotide encoding a response factor are integrated into the genome of said engineered yeast cells.

55. The engineered yeast cell of any one of claims 35-54, wherein said engineered yeast comprises Saccharomyces, such as Saccharomyces cerevisiae and/or Saccharomyces boulardii.

56. The engineered yeast cell of any one of claims 35-55, wherein said target molecule comprises a ligand, a protein, metabolite, a hormone, a nutrient, or small molecule, such as cortisol or histamine.

57. The engineered yeast cell of any one of claims 35-56, wherein said target molecule comprises cortisol or histamine.

58. The engineered yeast cell of any one of claims 35-57, wherein said engineered yeast cells produce an effective amount of said response factor in response to a threshold concentration of said target molecule.

59. The engineered yeast cell composition of claim 58, wherein when said target molecule is cortisol, said threshold concentration is at least 0.1 nM, at least 0.5 nM, at least 1 nM, at least 5 nM, at least 10 nM, at least 100 nM, at least 500 nM, at least 1 μM, or at least 5 μM.

60. The engineered yeast cell composition of claim 58, wherein when said target molecule is histamine, said threshold concentration is 0.1 nM, at least 0.5 nM, at least 1 nM, at least 5 nM, at least 10 nM, at least 100 nM, at least 500 nM, at least 1 μM, or at least 5 μM.

61. A pharmaceutical composition comprising the biosensing composition of any one of claims 1-26 or 34, the probiotic of any one of claims 27-33, or the engineered yeast cell of any one of claims 35-60, and a pharmaceutically acceptable carrier.

62. The probiotic of any one of claims 27-33 or the pharmaceutical composition of claim 61, wherein said probiotic or pharmaceutical composition is suitable for oral administration to a subject, such as a mammalian subject, such as a human.

63. A method of manufacturing a biosensing composition comprising a plurality of engineered yeast cells suitable for detecting, binding, detecting, and/or reducing the levels of a target molecule in a subject, said method comprising the steps of:
a) introducing into a yeast cell a nucleic acid molecule comprising a polynucleotide comprising a nucleotide sequence encoding a molecule responsive element comprising a binding domain that interacts with a target molecule,
b) introducing into said yeast cell a nucleic acid molecule comprising a polynucleotide comprising a nucleotide sequence encoding a response factor, and
c) culturing said engineered yeast cell under conditions sufficient to allow production of a yeast cell culture comprising a plurality of engineered yeast cells comprising said polynucleotide encoding said molecule responsive element and said polynucleotide encoding said response factor.

64. The method of claim 63, further comprising the step of selecting for yeast cells comprising said polynucleotide encoding said molecule responsive element and/or said polynucleotide encoding said response factor.

65. The method of claim 63 or 64, wherein at least 50%, at least 55%, at least 60%, at least 65%, at least 70%, at least 75%, at least 80%, at least 85%, at least 90%, at least 95%, or at least 99% of said engineered yeast cells within said plurality comprises said polynucleotide encoding said molecule responsive element and said polynucleotide encoding said response factor.

66. The method of any one of claims 63-65, wherein introduction of said nucleic acid molecule of step a) and/or step b) occurs simultaneously.

67. The method of any one of claims 63-65, wherein said introduction of said nucleic acid molecule of step a) and/or step b) occurs sequentially.

68. The method of any one of claims 63-67, wherein said yeast comprises Saccharomyces, such as Saccharomyces cerevisiae and/or Saccharomyces boulardii.

69. The method of any one of claims 63-68, wherein said molecule responsive element comprises a polypeptide.

70. The method of claim 69, wherein said polypeptide comprises an amino acid sequence comprising said binding domain.

71. The method of any one of claims 63-70, wherein said molecule responsive element comprises a nucleic acid molecule.

72. The method of any one of claims 69-71, wherein said polypeptide comprises a protein, such as a fusion protein, a transmembrane protein, a receptor, and/or a functional fragment or variant thereof.

73. The method of claim 72, wherein said receptor comprises a human glucocorticosteroid (GC) receptor, or a functional fragment or variant thereof having at least 80%, at least 85%, at least 90%, at least 95%, or at least 99% sequence identity to a human glucocorticosteroid (GC) receptor.

74. The method of claim 73, wherein said human glucocorticosteroid (GC) receptor comprises an amino acid sequence set forth as SEQ ID NO: 1, or a functional fragment or variant thereof having at least 80%, at least 85%, at least 90%, at least 95%, or at least 99% sequence identity to the amino acid sequence set forth as SEQ ID NO: 1; and/or
wherein said human glucocorticosteroid (GC) receptor is encoded by a nucleotide sequence set forth as SEQ ID NO: 2, or a variant thereof having at least 80%, at least 85%, at least 90%, at least 95%, or at least 99% sequence identity to the nucleotide sequence set forth as SEQ ID NO: 2.

75. The method of claim 72, wherein said receptor comprises a G-protein coupled receptor (GPCR) protein, or a functional fragment or variant thereof having at least 80%, at least 85%, at least 90%, at least 95%, or at least 99% sequence identity to a G-protein coupled receptor (GPCR) protein

76. The method of claim 75, wherein said G-protein coupled receptor (GPCR) protein is a human G-protein coupled receptor (GPCR) protein.

77. The method of claim 75 or 76, wherein said G-protein coupled receptor (GPCR) fusion protein, or functional fragment or variant thereof, comprises a histamine binding domain, such as a histamine binding ectodomain.

78. The method of any one of claims 75-77, wherein
human G-protein coupled receptor (GPCR) protein comprises an amino acid sequence set forth as SEQ ID NO: 3, or a functional fragment or variant thereof having at least 80%, at least 85%, at least 90%, at least 95%, or at least 99% sequence identity to the amino acid sequence set forth as SEQ ID NO: 3; and/or
wherein said human G-protein coupled receptor (GPCR) protein is encoded by a nucleotide sequence set forth as SEQ ID NO: 4, or a variant thereof having at least 80%, at least 85%, at least 90%, at least 95%, or at least 99% sequence identity to the nucleotide sequence set forth as SEQ ID NO: 4.

79. The method of any one of claims 63-78, further comprising the step of:
introducing into said yeast cell a polynucleotide comprising a nucleotide sequence encoding a polypeptide encoding a modified yeast GPA1 protein,
wherein said polypeptide:
a) comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence set forth as SEQ ID NO: 10;
b) comprises an amino acid sequence corresponding with residues 1-467 of SEQ ID NO: 10;
c) comprises an amino acid sequence comprising one or more than one amino acid substitution compared to SEQ ID NO: 10, wherein said one or more than one amino acid substitution is selected from the following: K468E, 1469Y, G470N, 1471L, and 1472V;
d) comprises an amino acid sequence comprising the following amino acid substitutions: K468E, I469Y, G470N, I471L, and I472V, compared to SEQ ID NO: 10; and/or
e) comprises an amino acid sequence comprising an amino acid sequence set forth as SEQ ID NO: 8, or a functional fragment or variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth as SEQ ID NO: 8; and/or
wherein said polynucleotide comprises a nucleotide sequence set forth as SEQ ID NO: 7, or a variant having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth as SEQ ID NO: 7.

80. The method of any one of claims 63-79, wherein said polynucleotide sequence encoding said response factor is operably fused to an activation domain.

81. The method of claim 80, wherein said activation domain comprises a promoter, such as a Ste-12-activated or a human glucocorticosteroid response element (HRE)-activated promoter.

82. The method of claim 81, wherein said response factor is produced upon binding of STE12 or GC receptor to said promoter.

83. The method of any one of any one of claims 63-82, wherein said engineered yeast comprises mutation and/or deletion in at least one of the following genes: STE2, FAR1, SST2, and MTA-A.

84. The method of any one of claims 63-83, wherein said nucleotide sequence encoding said molecule responsive element is codon-optimized for expression in a eukaryotic cell, such as a yeast cell.

85. The method of any one of claims 63-84, wherein said nucleotide sequence encoding molecule responsive element and said nucleotide sequence encoding said response factor are comprised within a single nucleic acid molecule.

86. The method of claim 85, wherein said nucleic acid molecule comprises a plasmid.

87. The method of any one of claims 63-84, wherein said nucleotide sequence encoding said molecule responsive element and/or said nucleotide sequence encoding a response factor are integrated into the genome of said engineered yeast cells.

88. The method of any one of claims 63-87, wherein said engineered yeast comprises Saccharomyces, such as Saccharomyces cerevisiae and/or Saccharomyces boulardii.

89. The method of any one of claims 63-88 wherein said target molecule comprises a ligand, a protein, metabolite, a hormone, a nutrient, or small molecule, such as cortisol or histamine.

90. The method of any one of claims 63-89, wherein said target molecule comprises cortisol or histamine.

91. The method of any one of claims 63-90, wherein said engineered yeast cells produce an effective amount of said response factor in response to a threshold concentration of said target molecule.

92. The method of claim 91, wherein when said target molecule is cortisol, said threshold concentration is at least 0.1 nM, at least 0.5 nM, at least 1 nM, at least 5 nM, at least 10 nM, at least 100 nM, at least 500 nM, at least 1 μM, or at least 5 μM.

93. The method of claim 91, wherein when said target molecule is histamine, said threshold concentration is 0.1 nM, at least 0.5 nM, at least 1 nM, at least 5 nM, at least 10 nM, at least 100 nM, at least 500 nM, at least 1 μM, or at least 5 μM.

94. The method of any one of claims 63-93, wherein said response factor comprises a postbiotic compound.

95. The method of any one of claims 63-94, wherein said biosensing compositions is formulated into the form of a capsule, gel, softgel, paste, tablet, powder, gummy, spray, aerosol, lozenge, effervescent tablet, orodispersible film, topical cream, ointment, suppository, emulsion, transdermal patch, or liquid.

96. The method of any one of claims 63-95, wherein said biosensing composition is formulated into a nutritional supplement, nutritional whole food, drink, sports drink, coffee, tea, water, mineral water, soda, carbonated beverage, oral hydration drink, mineral water, soup, replacement food, nutritional bar, nutritional consumable, gummy, tablet, confectionery, fermented or unfermented milk-based product, yogurt product, milk-based powder, enteral nutritional product, fermented or unfermented cereal-based product, chocolate, milk, yogurt, cheese, ice cream, baby and infant formula, cereal, animal feed, protein shake, protein powder, functional water, juice blend, smoothie, ready-to-drink (RTD) beverage, non-dairy milk, nut or seed butter, energy drink, functional shot, baked good, granola bar, snack bar, alcoholic beverage, frozen food, frozen meal, condiment, pasta, noodles, pet food, or pet treat.

97. The method of any one of claims 63-95, wherein said biosensing composition is formulated for administration to a plant or plurality of plants.

98. A method of binding, detecting, and/or reducing the levels of a target molecule in a subject in need thereof, said method comprising administering to said subject a therapeutic amount of the biosensing composition of any one of claims 1-26 or 34, the probiotic of any one of claims 27-33 or 62, or the pharmaceutical composition of claim 61 or 62.

99. A method of treating a disease, or disorder, or symptom associated with a disease or disorder, in a subject in need thereof, said method comprising administering to said subject a therapeutic amount of the biosensing composition of any one of claims 1-26 or 34, the probiotic of any one of claims 27-33 or 62, or the pharmaceutical composition of claim 61 or 62.

100. The method of claim 98 or 99, wherein said disease or disorder is a stress disorder, an inflammatory disorder, an autoimmune disorder, a disorder associated with microbiome dysbiosis, a hormonal imbalance disorder, an infection, a skin disorder, and/or a gastrointestinal or digestive disorder.

101. The method of any one of claims 98-100, wherein said subject comprises elevated cortisol levels or elevated histamine levels.

102. A method of maintaining a healthy biological function within a tissue of a subject, said method comprising administering to said subject a therapeutic amount of the biosensing composition of any one of claims 1-26 or 34, the probiotic of any one of claims 27-33 or 62, or the pharmaceutical composition of claim 61 or 62.

103. The method of any one of claims 98-102, wherein said molecule responsive element interacts with said target molecule.

104. The method of any one of claims 98-103, wherein upon interaction of said target molecule and said molecule responsive element, said molecule responsive element promotes increased expression of a gene encoding, or increased production and/or secretion of, the response factor.

105. The method of claim 104, wherein said increased production and/or secretion of the response factor contributes to reduced levels or reduced activity of said target molecule.

106. The method of any one of claims 98-105, wherein said target molecule comprises a ligand, a protein, metabolite, a hormone, a nutrient, or small molecule, such as cortisol or histamine.

107. The method of any one of claims 98-106, wherein said target molecule is present on the skin or in the esophagus, nasal tissue, mouth, vaginal tissue, lungs, stomach, small intestines, large intestines, digestive tract, or gastrointestinal tract of said subject.

108. The method of any one of claims 98-107, wherein levels of said target molecule are reduced in said subject.

109. The method of any one of claims 98-108, wherein said biosensing composition, probiotic, or pharmaceutical composition is administered at least once a day, at least one a week, at least twice a week, at least once a month, at least twice a month, at least once a year, or at least twice a year.

110. The biosensing composition of any one of claims 1-26 or 34, the probiotic of any one of claims 27-33 or 62, the engineered yeast cell of any one of claims 35-60, the pharmaceutical composition of claim 61, or the method of any one of claim 63-109, wherein said target molecule is produced by a/said subject.
